(19) United States
(12) Patent Application Publication
(10) Pub. No.: US 2025/0352972 A1
(43) Pub. Date: Nov. 20, 2025

(54) HIGHLY ACCURATE DE NOVO POLYNUCLEOTIDE SYNTHESIS

(71) Applicant: Twist Bioscience Corporation, South San Francisco, CA (US)

(72) Inventors: Bill James PECK, Santa Clara, CA (US); Mary NOE, San Francisco, CA (US); Stefan PITSCH, Stein am Rhein (CH); Patrick A. WEISS, Daly City, CA (US)

(73) Assignee: Twist Bioscience Corporation, South San Francisco, CA (US)

(21) Appl. No.: 19/233,645

(22) Filed: Jun. 10, 2025

Related U.S. Application Data
(63) Continuation of application No. 16/726,073, filed on Dec. 23, 2019, now Pat. No. 12,357,959.
(60) Provisional application No. 62/785,015, filed on Dec. 26, 2018.

(51) Int. Cl. B01J 19/00; C07C 53/02; C07C 53/08; C07C 53/12; C07C 53/122; C07C 53/134; C07D 213/16; C07D 213/74; C07D 233/58; C07H 21/04

(52) U.S. Cl. CPC B01J 19/0046; C07H 21/04; B01J 2219/00596; B01J 2219/00722; C07C 53/02; C07C 53/08; C07C 53/12; C07C 53/122; C07C 53/134; C07D 213/16; C07D 213/74; C07D 233/58

(57) ABSTRACT

Methods, systems, compositions, and devices for the manufacturing of high-quality building blocks, such as polynucleotides, are described herein. Processes described herein provide for efficient washing of residual reagents, solvents, or byproducts from previous synthetic steps to allow for the generation of polynucleotides with low error rates. Processes described herein also provide for reduction in deletion rates during chemical nucleic acid synthesis. Further, methods and devices described herein allow for the rapid construction and assembly of large libraries of highly accurate polynucleotides.

### CROSS-REFERENCE

This application claims the benefit of U.S. provisional patent application No. 62/785,015 filed on Dec. 26, 2018 which is incorporated by reference in its entirety.

### BACKGROUND

De novo gene synthesis is a powerful tool for basic biological research and biotechnology applications. While various methods are known for the design and synthesis of relatively short fragments in a small scale, these techniques often suffer from predictability, scalability, automation, speed, accuracy, and cost.

### BRIEF SUMMARY

Provided herein are systems, methods, and compositions for the efficient de novo synthesis of highly accurate and uniform polynucleotide libraries.

Provided herein are methods for polynucleotide synthesis, comprising: a) providing a structure comprising a surface; b) coupling at least one nucleoside to a polynucleotide attached to the surface; c) depositing an oxidizing solution on the surface; d) depositing a wash solvent on the surface, wherein the wash solvent comprises a ketone, an ester, an ether, a hydrocarbon, or a functional equivalent thereof; and c) repeating steps b-d to synthesize a plurality of polynucleotides. Further provided herein, the wash solvent comprises acetone, tetrahydrofuran, ethyl acetate, toluene, benzene, ethanol, or a combination thereof. Further provided herein, the wash solvent comprises a ketone, an ether, or a functional equivalent thereof. Further provided herein, the wash solvent comprises acetone, or a functional equivalent thereof. Further provided herein, the wash solvent is functionally equivalent to a primary constituent by volume of a previously contacted reagent solution. Further provided herein, one or more of steps b) to d) is followed by washing the surface with the wash solvent. Further provided herein, one or more steps b) to d) is followed by washing the surface with acetonitrile. Further provided herein, each step subsequent to the coupling step is followed by washing the surface with the wash solvent. Further provided herein, each step subsequent to the deblocking step is followed by washing the surface with the wash solvent. Further provided herein, the method further comprises depositing a capping solution on the surface, wherein capping prevents coupling of unblocked nucleosides. Further provided herein, the capping solution comprises an acid halide or an anhydride. Further provided herein, the capping solution comprises acetyl chloride or acetic anhydride. Further provided herein, the capping solution comprises an amine base. Further provided herein, each step subsequent to the capping step is followed by washing the surface with the wash solvent. Further provided herein, the method further comprises depositing a deblocking solution on the surface, wherein deblocking allows coupling of the polynucleotide to a nucleoside. Further provided herein, the at least one nucleoside comprises a phosphoramidite. Further provided herein, the at least one nucleoside comprises a 5′ blocking group. Further provided herein, the at least one nucleoside comprises a 3′ blocking group. Further provided herein, the structure is a plate, a tape, a belt, or a bead. Further provided herein, the method further comprises depositing a deblocking solution on the surface, wherein deblocking allows coupling of the polynucleotide to a nucleoside. Further provided herein, the method further comprises depositing a capping solution on the surface before or after depositing the oxidizing solution on the surface. Provided herein are methods further comprising depositing the capping solution on the surface before and after depositing the oxidizing solution on the surface. Further provided herein, are methods further comprising depositing a wash solvent after (i) coupling the at least one nucleoside to the polynucleotide attached to the surface; (ii) depositing the capping solution; and (iii) depositing the oxidizing solution. Further provided herein, the oxidizing solution comprises iodine. Further provided herein, the oxidizing solution comprises I₂ or iodine salts, and the I₂ or iodine salts have a greater solubility or increased rate of dissolution in the wash solvent compared to acetonitrile. Further provided herein, are methods wherein the oxidizing solution further comprises an amine base. Further provided herein, the amine base is selected from pyridine, lutidine, collidine, N-methyl morpholine, or a functional equivalent thereof. Provided herein are methods further comprising: providing predetermined sequences for a plurality of preselected polynucleotides before step (a); and assembling the plurality of preselected polynucleotides after step (e), wherein the wash solvent dissolves an active component or byproduct of the oxidizing solution. Further provided herein, the method further comprises additional washing before or after depositing the oxidizing solution on the surface, wherein washing comprises depositing the wash solvent on the surface. Further provided herein, the polynucleotide or nucleoside comprises DNA or RNA.

Provided herein are compositions for polynucleotide synthesis comprising: a) at least one base; b) at least one O-nucleophile; and c) at least one solvent. Further provided herein, the at least one base is selected from the group consisting of pyridine, lutidine, and collidine. Further provided herein, the at least one nucleophile is an O-nucleophile selected from group consisting of acetic acid, formic acid, propionic acid, methoxyacetic acid, phenoxyacetic acid, and water. Further provided herein, the O-nucleophile is selected from group consisting of acetic acid, and water. Further provided herein, the concentration of the O-nucleophile is 0.01-3M. Further provided herein, the concentration of the O-nucleophile is 0.1-0.5M. Further provided herein, the at least one solvent is acetonitrile, acetone, or THF. Further provided herein, the concentration of the at least one base is 0.01-3M. Further provided herein, the concentration of the at least one base is 0.1-0.5M.

Provided herein are methods of polynucleotide synthesis comprising: a) providing a structure comprising a surface; b) coupling at least one nucleoside to a polynucleotide attached to the surface; c) depositing an oxidizing solution on the surface; and d) repeating steps (b)-(c) to synthesize a plurality of polynucleotides, wherein the method comprises depositing a composition of any one of the compositions described herein. Further provided herein, depositing occurs during any of steps (a)-(d). Further provided herein, depositing occurs after step (b). Provided herein are methods further comprising at least one washing step with a wash solvent, wherein the wash solvent comprises acetone or THF.

Provided herein are compositions for polynucleotide synthesis comprising: a) at least one base; b) at least one O-nucleophile; c) at least one electrophile; and d) at least one solvent. Further provided herein, the at least one base is selected from the group consisting of pyridine, lutidine, and collidine. Further provided herein, the O-nucleophile is selected from group consisting of acetic acid, formic acid, propionic acid, methoxyacetic acid, phenoxyacetic acid, and water. Further provided herein, the O-nucleophile is selected from group consisting of acetic acid, methoxyacetic acid, phenoxyacetic acid, and water. Further provided herein, the concentration of the O-nucleophile is 0.01-3M. Further provided herein, the concentration of the O-nucleophile is 0.1-0.5M. Further provided herein, the electrophile is an anhydride, NHS ester, or acid halide. Further provided herein, the at least one electrophile is an anhydride or acid halide. Further provided herein, the anhydride is acetic anhydride. Further provided herein, the composition further comprises an activator. Further provided herein, the activator is N-methylimidazole or DMAP. Further provided herein, the concentration of the activator is 0.001-0.05M. Further provided herein, the at least one solvent is acetonitrile, acetone, or THF. Further provided herein, the concentration of the at least one base is 0.01-3M. Further provided herein, the concentration of the at least one base is 0.1-0.5M. Provided herein are methods of polynucleotide synthesis comprising: a)
providing a structure comprising a surface; b) coupling at least one nucleoside to a polynucleotide attached to the surface; c) depositing an oxidizing solution on the surface; d) depositing a capping solution on the surface, wherein the capping solution comprises the composition described herein; and e) repeating steps (b)-(d) to synthesize a plurality of polynucleotides. Further provided herein, the method further comprises at least one washing step comprising acetone or THF. Further provided herein, the nucleoside comprises a protected nucleoside phosphoramidite. Further provided herein, the nucleoside comprises a protected base. Further provided herein, the protected nucleoside phosphoramidite comprises a protected guanine or thymine. Further provided herein, the protected nucleoside phosphoramidite is selected from the group consisting of:

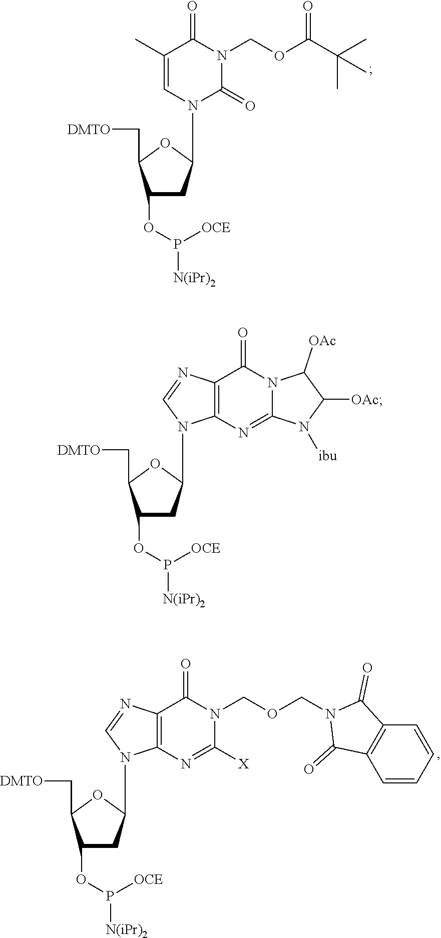

wherein X is —N═CHNR², and R is CH₃ or CH₂CH₃; and

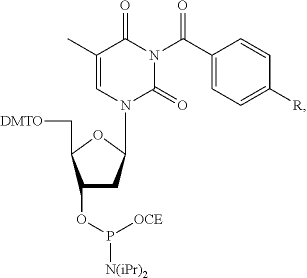

wherein R is H, OCH₃, F, or tert-butyl; wherein DMT is dimethoxytrityl; and CE is cyanoethyl.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a workflow for nucleoside phosphoramidite based polynucleotide synthesis.

FIG. 1B depicts a continuous loop arrangement for polynucleotide synthesis structures.

FIG. 1C depicts a reel-to-reel arrangement for polynucleotide synthesis structures.

FIG. 1D illustrates a plate configured for polynucleotide synthesis comprising 24 regions, or sub-fields, each having an array of 256 clusters.

FIG. 1E illustrates a closer view of the sub-field in FIG. 1D having 16×16 of clusters, each cluster having 121 individual loci.

FIG. 1F illustrates a detailed view of the cluster in FIG. 1D, where the cluster has 121 loci.

FIG. 2 depicts a schematic for the generation of polynucleotide libraries from cluster amplification.

FIG. 3 depicts a polynucleotide synthesis material deposition device.

FIG. 4 depicts a polynucleotide synthesis workflow.

FIG. 5 depicts a computer system.

FIG. 6 depicts a block diagram illustrating the architecture of a computer system.

FIG. 7 depicts a network configured to incorporate a plurality of computer systems, a plurality of cell phones and personal data assistants, and Network Attached Storage (NAS).

FIG. 8 depicts a multiprocessor computer system using a shared virtual address memory space.

FIG. 9A depicts block deletion and single base deletion rates across columns of clusters on a surface for polynucleotides synthesized using washing Conditions A with acetone.

FIG. 9B depicts block deletion and single base deletion rates across columns of clusters on a surface for polynucleotides synthesized using washing Conditions B with acetonitrile.

FIG. 9C depicts single base deletion rates across clusters on a surface for polynucleotides synthesized using washing Conditions A with acetone. Darker shaded circles indicate a higher deletion rate.

FIG. 9D depicts single base deletion rates across clusters on a surface for polynucleotides synthesized using washing Conditions B with acetonitrile. Darker shaded circles indicate a higher deletion rate.

FIG. 10 depicts overall error rates for three different sets of samples determined using Sanger sequencing.

FIG. 11A depicts polynucleotide error rates by error class (deletion, insertion, or mismatch) determined using Next Generation Sequencing (NGS).

FIG. 11B depicts polynucleotide error rates for various substitutions determined using Next-Generation Sequencing (NGS).

FIG. 11C depicts polynucleotide error rates for deletions determined using NGS.

FIG. 11D depicts polynucleotide error rates for insertions determined using NGS.

FIG. 11E depicts error rates for various error classes (insertions, mismatches, single deletions, and block deletions) at different base positions on a synthesized polynucleotide, determined using NGS. The base at position one is furthest from the synthesis surface, and therefore was added last to the synthesized polynucleotide chain.

FIG. 12A depicts representation uniformity, dropout rate, and total reads of synthesized polynucleotides at various positions on a synthesis surface. Lighter shaded squares on the surface indicate lower average read count, and darker shaded squares indicate higher average read count.

FIG. 12B depicts representation uniformity of a synthesized polynucleotide library.

FIG. 12C depicts representation uniformity of a synthesized polynucleotide library as a function of cluster (top) and device (bottom). Different levels of shading indicate individual clusters (top) or devices (bottom).

### DETAILED DESCRIPTION OF THE INVENTION

Provided herein are systems, methods, and compositions for the efficient de novo synthesis of highly accurate and uniform polynucleotide libraries. Further provided herein are methods comprising post-oxidation step washing with a solvent, wherein the solvent dissolves a primary reagent or reagent byproduct of the oxidation step.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence,” or “predetermined sequence” are used interchangeably. The terms mean that the sequence of the polymer is known and chosen before synthesis or assembly of the polymer. In particular, various aspects of the invention are described herein primarily with regard to the preparation of polynucleotides, the sequence of the oligonucleotide or polynucleotide being known and chosen before the synthesis or assembly of the polynucleotides.

Methods, systems, devices, and compositions described herein in various aspects comprise contacting a surface with a solvent or solvent mixture, variously described as a “wash,” “wash solvent,” “wash buffer,” “bath,” “cleaning solvent,” or “rinse”. A wash step in some cases is used to push, flush, purge, remove, exchange, or replace other reagent solutions (comprising unreacted reagents (active components), solvents, or reagent byproducts (chemical products resulting from the reaction of the reagents) or previous wash solvents that are in contact with a surface. A residual reagent solution or solvent is in some cases used in the step immediately prior to the wash step.

The term “functional equivalent” used herein in regard to solvents describes an alternative solvent or solvent mixture that possess similar properties. These properties are in some cases physical properties (including e.g., boiling point, melting point, heat of vaporization, viscosity, miscibility, solubility, density, purity, or other physical property). Similar properties also optionally include performance measures, for example, a solvent and a functional equivalent both provide a similar outcome (reduced error rate, increase in error rate uniformity, dissolution of reagents or reagent byproducts, or other outcome) when used with the methods, systems, compositions, and devices described herein.

Provided herein are methods and compositions for production of synthetic (i.e. de novo synthesized or chemically synthesized) polynucleotides. The term oligonucleotide, oligo, and polynucleotide are defined to be synonymous throughout. Libraries of synthesized polynucleotides described herein may comprise a plurality of polynucleotides collectively encoding for one or more genes or gene fragments. In some instances, the polynucleotide library comprises coding or non-coding sequences. In some instances, the polynucleotide library encodes for a plurality of cDNA sequences. Reference gene sequences from which the cDNA sequences are based may contain introns, whereas cDNA sequences exclude introns. Polynucleotides described herein may encode for genes or gene fragments from an organism. Exemplary organisms include, without limitation, prokaryotes (e.g., bacteria) and eukaryotes (e.g., mice, rabbits, humans, and non-human primates). In some instances, the polynucleotide library comprises one or more polynucleotides, each of the one or more polynucleotides encoding sequences for multiple exons. Each polynucleotide within a library described herein may encode a different sequence, i.e., non-identical sequence. In some instances, each polynucleotide within a library described herein comprises at least one portion that is complementary to sequence of another polynucleotide within the library. Polynucleotide sequences described herein may, unless stated otherwise, comprise DNA or RNA.

Provided herein are methods and compositions for production of synthetic (i.e. de novo synthesized) genes. Libraries comprising synthetic genes may be constructed by a variety of methods described in further detail elsewhere herein, such as PCA, non-PCA gene assembly methods or hierarchical gene assembly, combining (“stitching”) two or more double-stranded polynucleotides to produce larger DNA units (i.e., a chassis). Libraries of large constructs may involve polynucleotides that are at least 1, 1.5, 2, 3, 4, 5, 6, 7, 8, 9, 10, 15, 20, 30, 40, 50, 60, 70, 80, 90, 100, 125, 150, 175, 200, 250, 300, 400, 500 kb long or longer. The large constructs can be bounded by an independently selected upper limit of about 5000, 10000, 20000 or 50000 base pairs. The synthesis of any number of polypeptide-segment encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g., promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred to herein, may comprise at least one region encoding for exon sequence(s) without an intervening intron sequence found in the corresponding genomic sequence. Alternatively, the corresponding genomic sequence to a cDNA may lack an intron sequence in the first place.

### De Novo Polynucleotide Synthesis

Provided herein are methods for the synthesis of polynucleotides that typically involve an iterating sequence of the following steps: application of a protected monomer to a surface of a substrate feature to link with either the surface, a linker, or with a previously deprotected monomer; deprotection of the applied monomer so that it can react with a subsequently applied protected monomer; and application of another protected monomer for linking. One or more intermediate steps include oxidation and/or sulfurization. Optionally, intermediate steps include a capping step to block previously deprotected monomers which have not reacted with a protected monomer. Further provided herein methods comprising one or more wash steps with a solvent or solvent mixture (e.g., wash formulation) that follow at least one or all of the polynucleotide synthesis steps, such as an oxidation step, wherein the solvent or solvent mixture dissolves the primary reagent or reagent byproduct of the previous step.

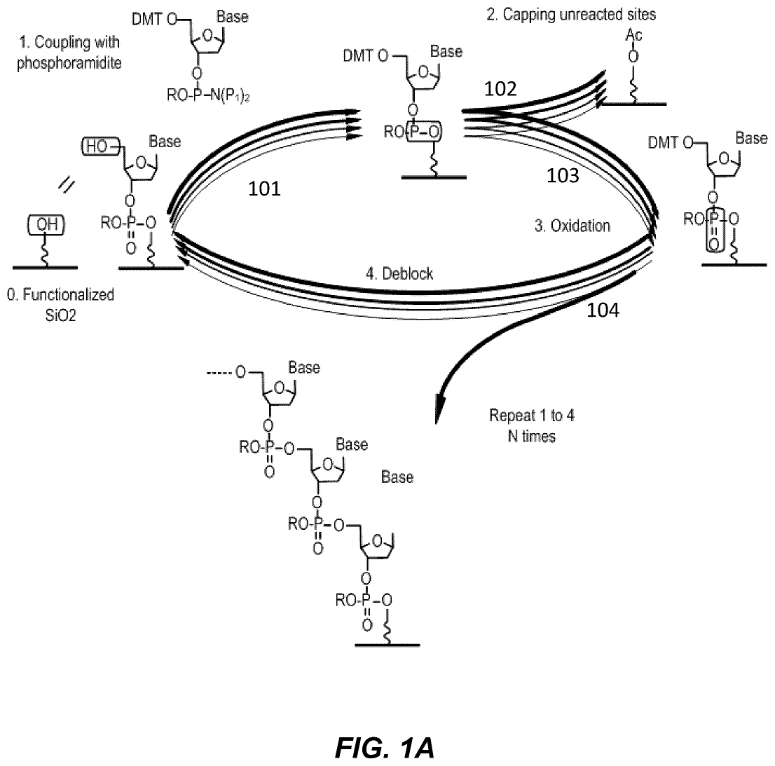

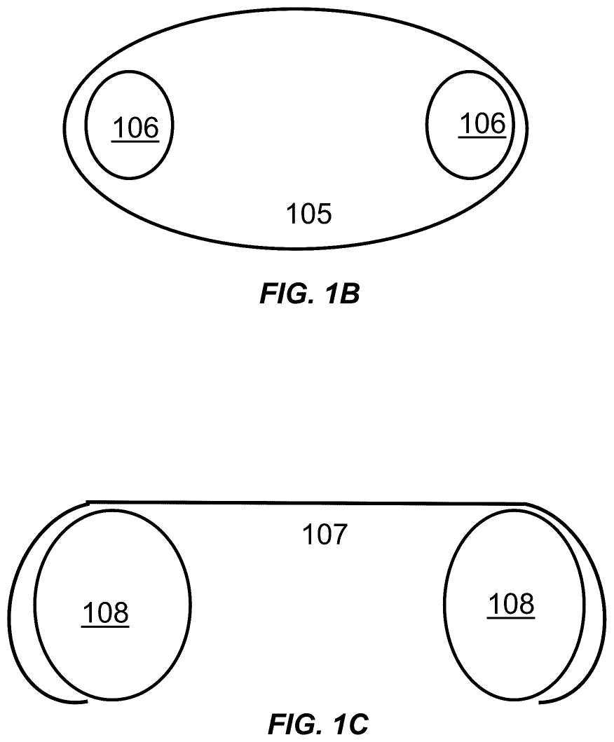

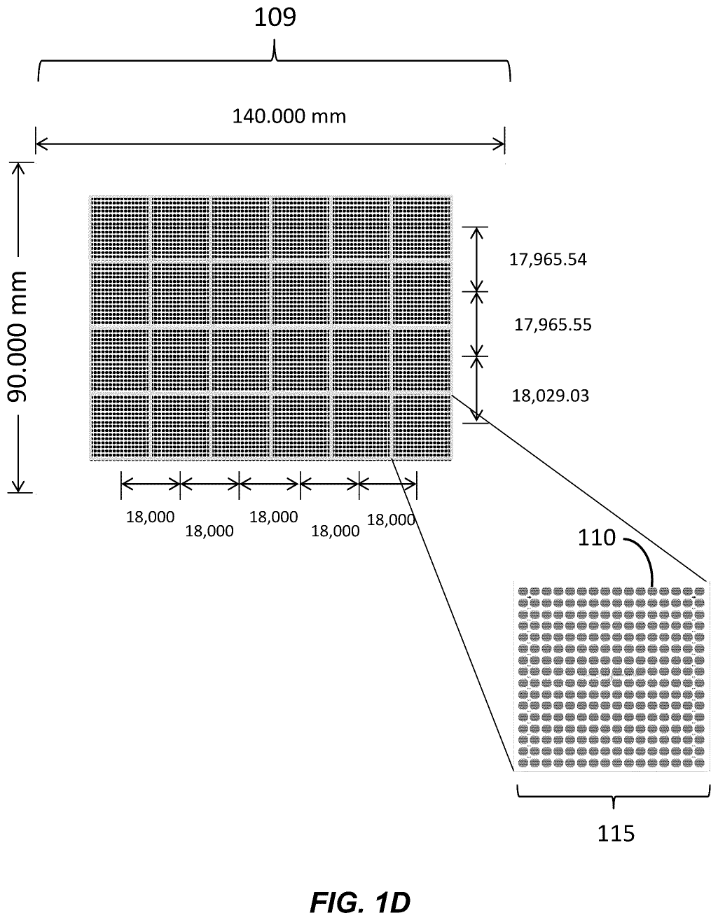

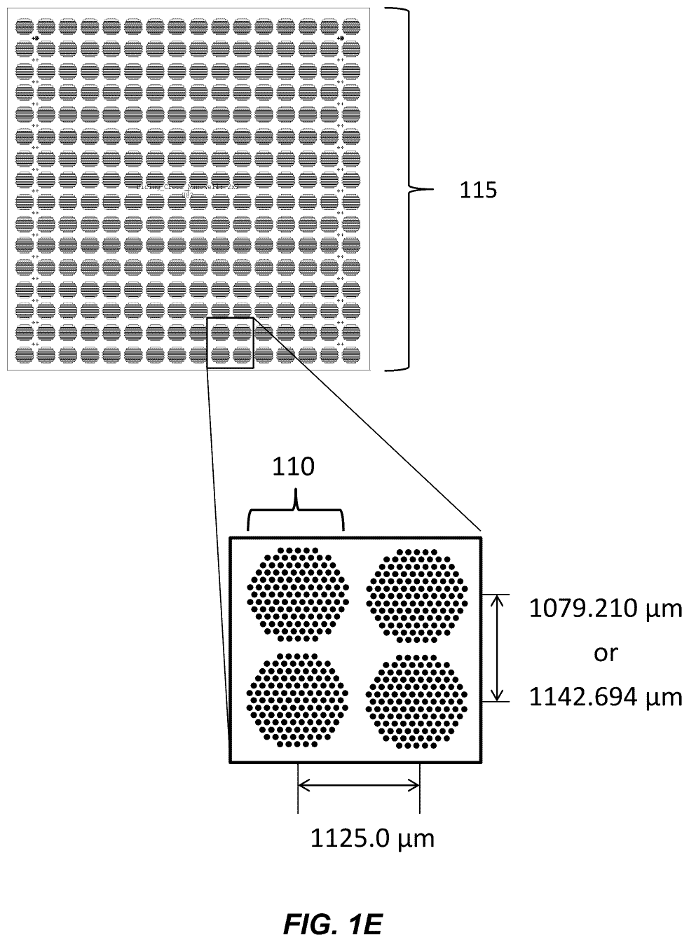

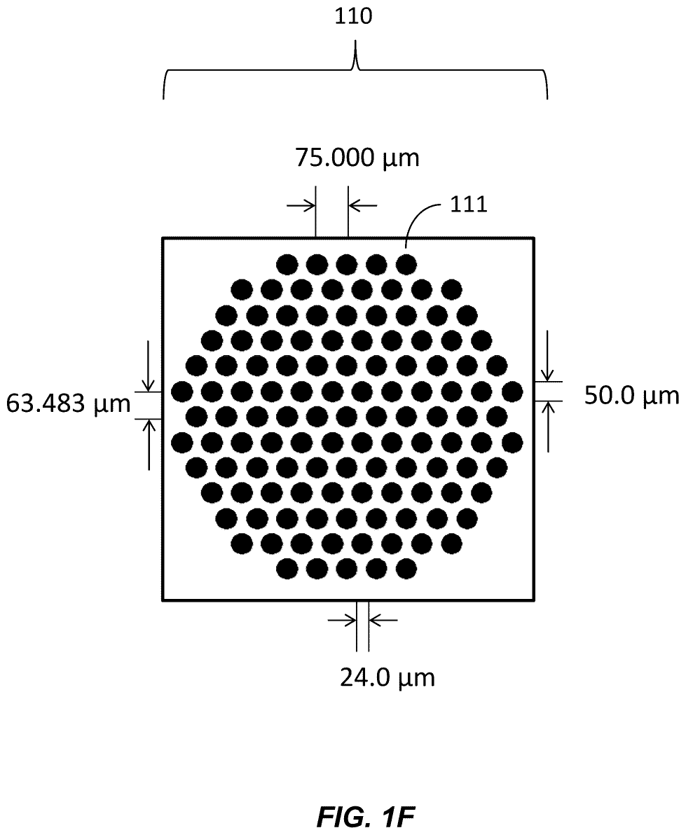

An exemplary synthetic scheme for polynucleotide synthesis in 3′ to 5′ direction is shown in FIG. 1. In Step 0, a surface is functionalized with a blocked nucleoside and then deblocked (not shown) to provide an unblocked nucleoside attached to the surface. The unblocked nucleoside is then washed 101 with a wash solvent. In Step 1, a 5′ DMT protected phosphoramidite nucleoside is coupled with the unblocked nucleoside on the surface to form a phosphite ester, resulting in a polynucleotide that is extended by one base. The surface is then washed 102 with a wash solvent, and a capping reagent (or reagents) is added to block all unreacted 5′ OH groups on the surface with an acetate group. The surface is washed after capping 103, and further subjected to oxidation in Step 3 to generate a phosphate ester. The phosphate ester is washed 104 with a wash solvent, and deblocked by removal of the DMT group in Step 4 to generate an unblocked polynucleotide. The unblocked polynucleotide is washed 101 with a wash solvent, and Steps 1-4 are repeated to synthesize the polynucleotide. Alternatively, polynucleotides are synthesized in a 5′ to 3′ direction, wherein 3′ blocked phosphoramidite nucleotides are coupled to 3′ OH position of the growing polynucleotide chain.

Further provided herein are methods comprising wash steps that are executed before or after another step in de novo synthesis of polynucleotides, for example, a wash step is executed after surface preparation, after phosphoramidite coupling, after oxidation, after capping, or after deblocking. Wash steps often are used to remove residual reagents, solutions, reaction byproducts, or solvents from a previous synthetic step or a previous wash. For example, in an oxidation step comprising iodine and an amine base, a subsequent wash step comprises a solvent that dissolves a primary component of the reagent (remaining iodine or amine base), or a primary byproduct of said reagent (an iodide salt of an amine base). A wash step is often executed before one or more steps, such as before surface preparation, before phosphoramidite coupling, before oxidation, before capping, or before cleavage. Multiple wash steps are often used during polynucleotide synthesis, such as a plurality of wash steps separated by additional steps synthetic steps, or sequential wash steps. A wash step is in some instances executed after a deblocking step. A wash step is in some instances executed after a coupling step. A wash step is in some instances executed after an oxidation step. A wash step is in some instances executed after a capping step. A wash step is in some instances executed after a deblocking step, a coupling step, an oxidation step, and a capping step. A wash step in some instances comprises washing with a solvent or a mixture of solvents. For a given method, a single solvent or a plurality of different solvents or solvent mixtures are in some instances used for each individual wash step, or for all wash steps in the method. Wash steps in between synthesis steps are optionally omitted in some instances. In some instances, wash steps are performed with wash solvents comprising one or more solutes.

Further provided herein are methods wherein following addition of a nucleoside phosphoramidite, and optionally after capping and one or more wash steps, the substrate-bound growing polynucleotide is oxidized with an oxidizing solution. The oxidation step comprises oxidizing the phosphite triester into a tetracoordinated phosphate triester, a protected precursor of the naturally occurring phosphate diester internucleoside linkage. The oxidizing solution often comprises one or more chemical components. For example, an oxidizing solution comprises components such as one or more solvents (such as acetonitrile, acetone, THF or other solvent), and one or more oxidants or catalysts as active components or reagents. Oxidants variously comprise I₂, peroxides (e.g., hydrogen peroxide, mCPBA, TBHP, etc.), dioxiranes, or other oxidant known in the art capable of oxidizing a phosphate triester. Oxidation is sometimes carried out under anhydrous conditions using tert-butyl hydroperoxide or (1S)-(+)-(10-camphorsulfonyl)-oxaziridine (CSO). In some instances, the primary constituent (by mass) of an oxidizing solution is a solvent or solvent mixture. In some instances, the primary reagent of an oxidation solution is iodine. In some instances, oxidation of the growing polynucleotide is achieved by treatment with iodine and water, optionally in the presence of a weak base catalyst such as an amine base (e.g., pyridine, lutidine, collidine, N-methyl morpholine, or other amine base), or other weak base.

Washes with one or more solvents are often used to remove remaining oxidant or oxidation byproducts after an oxidation step. Without being bound by theory, solvent or solvent mixtures in some cases increases solubility of the oxidant, increase solubility of byproducts generated by the oxidant after oxidation, or in some instances react with the solvent to form a different oxidant. Alternately or in combination, a solvent or solvent mixture in some cases increases the rate of dissolution of the oxidant, or increases the rate of dissolution of byproducts generated by the oxidant after oxidation. Oxidation byproducts variously comprise salts, salts of a weak base (such as an amine), iodide salts, or reaction products of the oxidant and the solvent. In some instances, solvents that dissolve pyridinium iodide or other amine base salts of iodine are used as wash solvents following oxidation. In some instances, oxidation byproducts comprise iodine salts of pyridine, lutidine, collidine, N-methyl morpholine, or other weak base. The choice of solvent in some instances depends on the choice of oxidant; for example, an oxidizing solution comprises an oxidant I₂ and a solvent comprising acetone.

Reactions can occur at the O6 oxygen atom of guanosine and at the O4 oxygen atoms of thymidine/uridine nucleotides. The resulting activated nucleotide derivatives can be further modified by oxidation and nucleophilic aromatic substitution with additional reagents. In some instances, the activated nucleotide derivatives can be further modified by reagents used in capping, such as N-methylimidazole or DMAP. In some instances, the activated nucleotide derivatives can be further modified by reagents used in oxidation, such as pyridine. In some instances, the activated nucleotide derivatives can be further modified by reagents used in deprotection, such as methylamine, ammonia, or ethylenediamine. Products obtained after oxidation and capping can also react with amines used for deprotections to further generate unwanted products. The reaction between phosphoramidites with O-containing nucleotides can lead to N-substituted guanosine and thymidine/uridine nucleotides, which can form Watson-Crick base pairs with thymidine/uridine and guanosine nucleotides, respectively. The final consequences of undesired reactivity are G→A and T/U→C mutations.

Further provided herein are wash solutions that remove unwanted G→A and T/U→C mutations resulting from phosphoramidite-coupling to nucleobases by selectively removing undesired adducts directly after the coupling step and before the oxidation step. In some instances, wash solutions improve synthesis uniformity (e.g., yields, error rates, or other performance metric) across a solid support (e.g., a chip). The wash solutions of the disclosure in some instances comprise a solvent, a base, and an O-nucleophile. In some instances, the solvent is acetonitrile or THF. In some instances, the base is pyridine, lutidine, or collidine. In some instances, the O-nucleophile is water or an organic acid (such as acetic acid, formic acid, propionic acid). In some instances, the O-nucleophile is water. In some instances, a wash solution comprises THF, pyridine, water, or a combination thereof. In some instances, reagent concentration in such wash solutions is measured by ratios of various components. In some instances, a wash solution comprises a ratio of volumes. In some instances, a wash solution comprises a solvent, a base, and an O-nucleophile in a ratio of about 95:3:1, 90:7:3, 80:10:10, 80:20:10, 85:10:5, or 70:20:10. In some instances, a wash solution comprises a solvent, a base, and an O-nucleophile in a ratio of 60-90% solvent, 5-30% base, and 5-30% O-nucleophile. In some instances, a wash solution comprises a solvent, a base, and an O-nucleophile, wherein the ratio of base to O-nucleophile is at least about 0.5:1, 1:1, 1.5:1, 2:1, 2.5:1, 3:1, 4:1, 5:1, 7:1, 9:1, 10:1, 12:1, or 15:1. In some instances, a wash solution comprises a solvent, a base, and an O-nucleophile, wherein the ratio of base to O-nucleophile is about 0.5:1-15:1, 0.5:1-3:1, 1:1-5:1, 1:1-9:1, or 2:1-12:1. In some instances, reagent concentration is measured by molarity (M=mol/L). In some instances, the O-nucleophile concentration is about 0.01M, 0.02M, 0.05M, 0.08M, 0.1M, 0.2M, 0.5M, 0.8M, 1.0M, 1.2M, 1.2M, 1.5M, 1.8M, 2M, 2.5M, or about 3M. In some instances, the O-nucleophile concentration is no more than 0.01M, 0.02M, 0.05M, 0.08M, 0.1M, 0.2M, 0.5M, 0.8M, 1.0M, 1.2M, 1.2M, 1.5M, 1.8M, 2M, 2.5M, or no more than 3M. In some instances, the O-nucleophile concentration is at least 0.01M, 0.02M, 0.05M, 0.08M, 0.1M, 0.2M, 0.5M, 0.8M, 1.0M, 1.2M, 1.2M, 1.5M, 1.8M, 2M, 2.5M, or at least 3M. In some instances, the O-nucleophile concentration is 0.01-0.1M, 0.01-0.5M, 0.01-1.5M, 0.5-2M, 0.5-1.0M, 0.2-1.2M, 0.8-2.0M, or 0.5-1M. In some instances, the base concentration is about 0.01M, 0.02M, 0.05M, 0.08M, 0.1M, 0.2M, 0.5M, 0.8M, 1.0M, 1.2M, 1.2M, 1.5M, 1.8M, 2M, 2.5M, or about 3M. In some instances, the base concentration is no more than 0.01M, 0.02M, 0.05M, 0.08M, 0.1M, 0.2M, 0.5M, 0.8M, 1.0M, 1.2M, 1.2M, 1.5M, 1.8M, 2M, 2.5M, or no more than 3M. In some instances, the base concentration is at least 0.01M, 0.02M, 0.05M, 0.08M, 0.1M, 0.2M, 0.5M, 0.8M, 1.0M, 1.2M, 1.2M, 1.5M, 1.8M, 2M, 2.5M, or at least 3M. In some instances, the base concentration is 0.01-0.1M, 0.01-0.5M, 0.01-1.5M, 0.5-2M, 0.5-1.0M, 0.2-1.2M, 0.8-2.0M, or 0.5-1M.

Provided herein are chemical polynucleotide synthesis methods wherein a wash solvent following an oxidation step comprises a solvent with high solubility for oxidant I₂ or iodine salts. Exemplary solvents with high solubility for oxidant I₂ or iodine salts are listed in Table 1. Provided herein are chemical polynucleotide synthesis methods wherein a wash solvent following the oxidation reaction comprises a solvent listed in Table 1. In some instances, a wash step following the oxidation reaction comprises use of a solvent comprising THF, acetone, or other solvent described herein. In some instances, a wash step following the oxidation reaction comprises use of a solvent comprising acetone. In some instances, a wash step following the oxidation reaction comprises use of a solvent comprising a mixture of acetone and THF. In some instances, the same solvent selected for use as a wash after oxidation reaction is also used as a washing reagent following additional reactions in the chemical synthesis workflow. In some instances, at least one wash step comprising THF, acetone, or another solvent described herein, is employed after an oxidation reaction, and all other wash steps in the chemical polynucleotide synthesis workflow comprise deposition of acetonitrile. Further described herein are methods wherein a washing solvent is used for its ability to dissolve iodine or iodine salts. Exemplary solubilities of iodine in various solvents used with the methods described herein at various temperatures are listed in Table 1.

TABLE 1
Solvents and associated solubility of iodine

| Solvent | Solubility in g/mL (at Deg C.)* |
|---|---|
| Ethyl alcohol | 0.1618 (8) |
| Toluene | 0.1012 (8) |
| Ethyl Acetate | 0.987 (8) |
| Benzene | 0.893 (8) |
| Chloroform | 0.269 (8) |
| Carbon Disulfide | 16.49 (25); 21.63 (35) |
| Carbon tetrachloride | 0.02603 (35) |
| 2,2-dimethyl butane | 1.369 (25); 1.989 (35) |
| ethyl alcohol | 21.48 (25); 24.51 (35) |
| ethyl ether | 25.18 (25); 28.50 (35) |
| p-Xylene | 16.56 (25); 20.11 (35) |
| Mesitylene | 20.27 (25) |
| Perfluoroheptane | 0.0119 (25); 0.0179 (35) |

*Note
temperature in degrees Celsius are in parentheses.

Further described herein are methods wherein the coupling comprises use of protected nucleoside phosphoramidites, such as base protected nucleoside phosphoramidites. In some instances, phosphoramidite building blocks give rise to unwanted reactions at the nucleobases, leading to branching and mutations. In some instances, methods and reagents block the reaction of guanosine and thymidine/uridine nucleotides with activated phosphoramidites. After activation, phosphoramidites are highly reactive (electrophilic) reagents which preferentially react with oxygen nucleophiles due to the strength of the thereby resulting P—O bonds. During reaction with 5′—OH groups of growing nucleic acid strands, the desired chain elongation reaction takes place. To some extent, however, this reaction in some instances also occurs at oxygen atoms O6 and O4 of guanosine and thymidine/uridine nucleotides, respectively. The resulting activated nucleotide derivative may, at least partially, be further modified by oxidation and nucleophilic aromatic substitution with N-methylimidazole (used in capping), DMAP (used also in capping), pyridine (used in oxidation) and various amines used in deprotection (e.g., methylamine, ammonia or ethylenediamine). The products obtained after oxidation and capping in some instances also react with the various amines used for deprotection. The above-mentioned reaction of phosphoramidites with O-containing nucleotides will often lead to N-substituted guanosine and thymidine/uridine nucleotides able to form Watson-Crick base pairs with thymidine/uridine and guanosine nucleotides, respectively. In context with duplex formation, the consequence is an apparent G→A and T/U→C mutation, respectively. In the context of the preparation of high-quality (e.g., low error-rate) polynucleotides, a minimal number of such mutations are desirable. Nucleic acid syntheses in flow cells are commonly carried out with an extremely large excess of activated phosphoramidites. As a result, only neglectable amounts of these expensive reagents are actually consumed. To increase efficiency and minimize chemical waste, it is in some instances desirable to use these activated phosphoramidite solutions in consecutive flow cell reactions. However, since activated G and T/U phosphoramidites undergo side-reactions described above, the longer these solutions are exposed to the growing polynucleotide, an increasing amount of branching and mutations are in some instances introduced. In some instances, this reactivity prevents the utilization of money and waste saving multiple-coupling/phosphoramidite recycling strategies in high quality nucleic acid syntheses. In some instances, undesired side reactions are partially or fully suppressed by the attachment of protecting groups which are stable during the synthesis of the polynucleotide, but can be cleaved at the end, preferentially under the same conditions as the other protecting groups present in the primary synthesis product (protected and immobilized polynucleotide). In some instances, a protected nucleoside phosphoramidite base comprises a protected nitrogen atom. In some instances, the protected nucleoside phosphoramidite base comprises cytosine, adenine, thymine, uracil, or guanine. In some instances, the protected nucleoside phosphoramidite base comprises thymine or guanine. Exemplary protected dT and dG phosphoramidite building blocks A-I containing a protecting group at nitrogen atoms N3 and N1, respectively, are shown below:

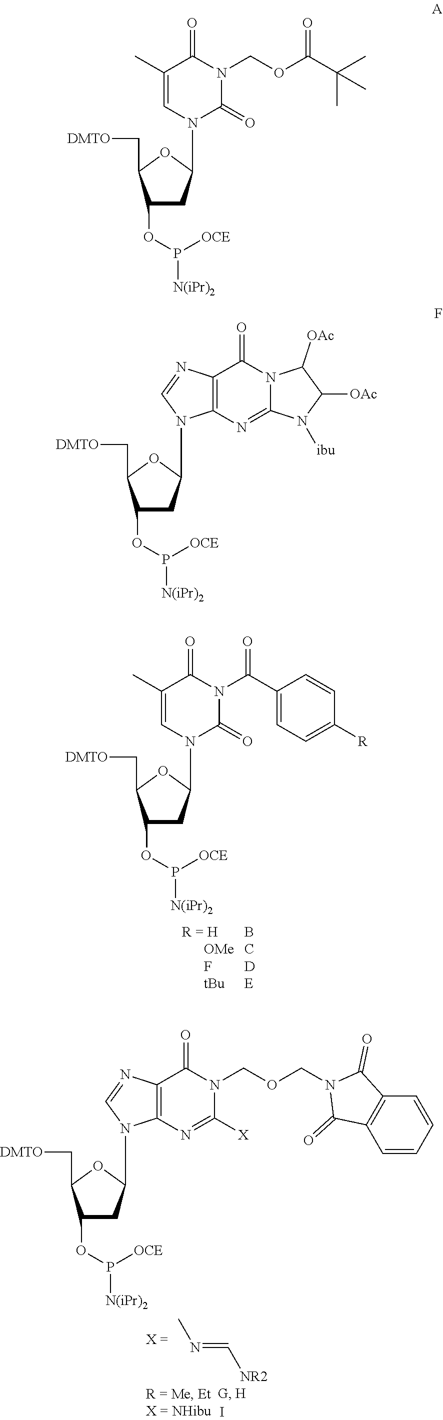

Further described herein are methods wherein following coupling, phosphoramidite polynucleotide synthesis methods comprise a capping step. In a capping step, the growing polynucleotide is treated with a capping agent. A capping step generally serves to block unreacted substrate-bound 5′—OH groups after coupling from further chain elongation, preventing the formation of polynucleotides with internal base deletions. Further, phosphoramidites activated with 1H-tetrazole often react, to a small extent, with the O6 position of guanosine. Without being bound by theory, upon oxidation with I₂/water, this side product, possibly via O6-N7 migration, undergoes depurination. The apurinic sites can end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the substrate is optionally washed. The capping reaction in DNA synthesis blocks non-coupled DNA fragments from being further elongated in later coupling steps, which suppresses the formation of n−1mer sequences. Capping reactions are carried out by coupling reactive phosphoramidites (“Unicap”) or by reacting a carboxylic acid anhydride in the presence of a base and an activator. In some instances, the carboxylic acid anhydride is acetic anhydride. In some instances, the base is lutidine. In some instances, the activator is N-methylimidazole or DMAP). Capping by reacting a carboxylic acid anhydride in the presence of a base and an activator can result in G→A and T→C mutation reactions, as a result of an electrophilic activation of the O6 and O4 oxygen atoms of guanosine and thymidine nucleotides, respectively, by acylation. The resulting activated nucleotide derivatives can then be further modified by nucleophilic aromatic substitution with N-methylimidazole or DMAP. The further modified products can react with various amines used for deprotection, which results in the formation of N-substituted guanosine and thymidine nucleotides able to form Watson-Crick base pairs with thymidine and guanosine nucleotides (i.e., G→A and T/U→C mutations).

Further provided herein are capping formulations that inhibit the formation of unwanted G→A and T→C mutations resulting from carboxylic acid-promoted activation of nucleobases. The capping formulations of the disclosure comprise a solvent, base, anhydride, and nucleophile. In some instances, the capping formulations comprise a solvent, base, anhydride, and O-nucleophile. In some instances, the capping formulations of the disclosure comprise a solvent, such as acetonitrile or THF. In some instances, the capping formulations of the disclosure comprise a base, such as lutidine or collidine. In some instances, the capping formulations of the disclosure comprise an anhydride, such as acetic anhydride. In some instances, the capping formulations of the disclosure comprise an activator, such as N-methylimidazole or DMAP. In some instances, the capping formulations of the disclosure comprise an O-nucleophile, such as acetic acid, methoxyacetic acid, or phenoxyacetic acid. A capping solution often comprises components such as one or more solvents (such as acetonitrile, acetone, THF or other solvent), one or more capping reagents, and one or more activators (N-methyl imidazole or other activator known in the art). Capping reagents variously comprise acid halides (e.g., acetyl chloride, or other acid halide), anhydrides (e.g., acetic anhydride, or other anhydride) or other capping reagent known in the art capable of reacting with 5′ OH of an unprotected nucleobase. In some instances, the primary constituent (by mass) of a capping solution is a solvent or solvent mixture. In some instances, one or more washes with one or more solvents is used to remove remaining capping reagents or activators after a capping step. Without being bound by theory, solvent or solvent mixtures in some cases increases solubility of capping reagents or activator, increases solubility of byproducts generated by the capping reagents, activator, or other reagent, or in some instances reacts with the capping reagent or activator to form a different capping or activating reagent. Alternately or in combination, a solvent or solvent mixture in some cases increases the rate of dissolution of the capping reagent or activator, or increases the rate of dissolution of byproducts generated by the capping reagent or activator. The choice of solvent in some instances depends on the choice of capping reagent or activator; for example, a capping solution comprises acetic anhydride and a solvent comprising tetrahydrofuran. Any combination of capping reagents activator, or other reagent and solvent is in some instances used with the methods described herein. In some instances, two or more capping solutions are prepared, and then mixed during polynucleotide synthesis during a capping step. In some instances, a first capping solution comprises a solvent and an activator. In some instances, a first capping solution comprises acetonitrile/n-methylimidazole in a 90:10 ratio. In some instances, a second capping solution comprises a solvent, base, and capping reagent. In some instances, a second capping solution comprises THF/lutidine/acetic anhydride in an 80:10:10 ratio. In some instances, the first and the second capping solutions are mixed together during the capping step. In some instances, a capping solution comprises a ratio of volumes. In some instances, a capping solution comprises a solvent, a base, and a capping reagent in a ratio of about 95:3:1, 90:7:3, 80:10:10, 80:20:10, 85:10:5, or 70:20:10. In some instances, a wash solution comprises a solvent, a base, and an a capping reagent in a ratio of 60-90% solvent, 5-30% base, and 5-30% capping reagent. In some instances, a wash solution comprises a solvent, a base, and a capping reagent, wherein the ratio of base to a capping reagent is at least about 0.5:1, 1:1, 1.5:1, 2:1, 2.5:1, 3:1, 4:1, 5:1, 7:1, 9:1, 10:1, 12:1, or 15:1. In some instances, a wash solution comprises a solvent, a base, and a capping reagent, wherein the ratio of base to a capping reagent is about 0.5:1-15:1, 0.5:1-3:1, 1:1-5:1, 1:1-9:1, or 2:1-12:1. In some instances, reagent concentration is measured by molarity (M=mol/L). In some instances, the O-nucleophile concentration is about 0.01M, 0.02M, 0.05M, 0.08M, 0.1M, 0.2M, 0.5M, 0.8M, 1.0M, 1.2M, 1.2M, 1.5M, 1.8M, 2M, 2.5M, or about 3M. In some instances, the O-nucleophile concentration is no more than 0.01M, 0.02M, 0.05M, 0.08M, 0.1M, 0.2M, 0.5M, 0.8M, 1.0M, 1.2M, 1.2M, 1.5M, 1.8M, 2M, 2.5M, or no more than 3M. In some instances, the O-nucleophile concentration is at least 0.01M, 0.02M, 0.05M, 0.08M, 0.1M, 0.2M, 0.5M, 0.8M, 1.0M, 1.2M, 1.2M, 1.5M, 1.8M, 2M, 2.5M, or at least 3M. In some instances, the O-nucleophile concentration is 0.01-0.1M, 0.01-0.5M, 0.01-1.5M, 0.5-2M, 0.5-1.0M, 0.2-1.2M, 0.8-2.0M, or 0.5-1M. In some instances, the base concentration is about 0.01M, 0.02M, 0.05M, 0.08M, 0.1M, 0.2M, 0.5M, 0.8M, 1.0M, 1.2M, 1.2M, 1.5M, 1.8M, 2M, 2.5M, or about 3M. In some instances, the base concentration is no more than 0.01M, 0.02M, 0.05M, 0.08M, 0.1M, 0.2M, 0.5M, 0.8M, 1.0M, 1.2M, 1.2M, 1.5M, 1.8M, 2M, 2.5M, or no more than 3M. In some instances, the base concentration is at least 0.01M, 0.02M, 0.05M, 0.08M, 0.1M, 0.2M, 0.5M, 0.8M, 1.0M, 1.2M, 1.2M, 1.5M, 1.8M, 2M, 2.5M, or at least 3M. In some instances, the base concentration is 0.01-0.1M, 0.01-0.5M, 0.01-1.5M, 0.5-2M, 0.5-1.0M, 0.2-1.2M, 0.8-2.0M, or 0.5-1M. In some instances, the activator concentration is about 0.001M, 0.002M, 0.005M, 0.008M, 0.01M, 0.02M, 0.05M, 0.08M, 0.1M, 0.12M, 0.15M, 0.18M, 0.2M, 0.3M, or about 0.5M. In some instances, the activator concentration is at least 0.001M, 0.002M, 0.005M, 0.008M, 0.01M, 0.02M, 0.05M, 0.08M, 0.1M, 0.12M, 0.15M, 0.18M, 0.2M, 0.3M, or at least 0.5M. In some instances, the activator concentration is no more than 0.001M, 0.002M, 0.005M, 0.008M, 0.01M, 0.02M, 0.05M, 0.08M, 0.1M, 0.12M, 0.15M, 0.18M, 0.2M, 0.3M, or no more than 0.5M. In some instances, the activator concentration is 0.001-0.01M, 0.001-0.05M, 0.001-0.15M, 0.05-0.2M, 0.005-0.02M, 0.01-0.1M, 0.08-0.2M, or 0.05-0.1M. In some instances, a capping step is performed following oxidation. In some instances a capping step is performed prior to oxidation. In some instances a capping step is performed prior to oxidation, and after oxidation. In some methods, a wash step is performed after oxidation. A second capping step allows for substrate drying, as residual water from oxidation that may persist can inhibit subsequent coupling. Following oxidation, the substrate and growing polynucleotide are optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including, but not limited to, 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

Further provided herein are methods comprising an elongation step that utilizes a coupling solution, wherein the coupling solution comprises one or more chemical components. For example, a coupling solution comprises components such as one or more solvents (such as acetonitrile, acetone, THF or other solvent), one or more monomers, and one or more activators (tetrazole or other activator known in the art). Monomers variously comprise phosphoramidite nucleosides, chlorophosphites, H-phosphonates, phosphodiesters, phosphotriesters, or other activated nucleoside known in the art capable of reacting with an unprotected monomer. In some instances, the primary constituent (by mass) of a coupling solution is a solvent or solvent mixture. In some instances, one or more washes with one or more solvents is used to remove remaining monomer, activator, or byproduct after a coupling step. Without being bound by theory, solvent or solvent mixtures in some cases increases solubility of monomer or activator, increases solubility of byproducts generated by the coupling reagents (monomers, activators, other reagent), or in some instances reacts with the solvent to form a different coupling or activating reagent. Alternately or in combination, a solvent or solvent mixture in some cases increases the rate of dissolution of the monomer or activator, or increases the rate of dissolution of byproducts generated by the monomer or activator. The choice of solvent in some instances depends on the choice of monomer or activator; for example, a coupling solution comprises a monomer phosphoramidite and a solvent comprising tetrahydrofuran. Any combination of coupling reagents (monomer, activator, or other reagent) and solvent is in some instances used with the systems, methods and compositions described herein. Polynucleotide synthesis methods used herein often comprise 1, 2, 3 or more sequential coupling steps.

Prior to coupling, the nucleoside bound to the substrate is often de-protected by removal of a protecting group, where the protecting group functions to prevent polymerization. Inability to completely remove one or more protecting groups in some instances leads to errors in polynucleotide synthesis products. In some instances, coupling steps are repeated two or more times without removal of a protecting group. Additional steps include but are not limited to capping, oxidation, or cleavage.

Further provided herein are methods comprising a deblocking (or deprotecting) step that utilizes a deblocking solution, wherein the deblocking solution comprises one or more chemical components. The deblocking solution often is used to remove protecting groups on 5′ OH of a polynucleotide or nucleotide. 5′ OH protecting groups are well known in the art, and, in some cases comprise trityl, DMT (4,4′-dimethoxytrityl), or other protecting group (including triarylmethyl, triphenylmethyl, or other group) wherein removal of the protecting group does not otherwise cleave or modify the polynucleotide. In some instances, a deblocking solution comprises components such as one or more solvents (such as acetonitrile, acetone, THF, toluene, or other solvent), and one or more deblocking reagents. Deblocking reagents variously comprise acids (trifluoracetic acid, or other acid), bases, light, heat, enzymes, or other reagent known in the art capable of removing a 5′ OH protecting group. In some instances, the primary constituent (by mass) of a deblocking solution is a solvent or solvent mixture. In some instances, one or more washes with one or more solvents is used to remove remaining deblocking reagent after a deblocking step. Without being bound by theory, solvent or solvent mixtures in some cases increases solubility of deblocking reagents, increases solubility of byproducts generated by the deblocking reagents (cleaved protecting groups), or in some instances reacts with the solvent to form a different deblocking reagent. Alternately or in combination, a solvent or solvent mixture in some cases increases the rate of dissolution of the deblocking reagent, or increases the rate of dissolution of byproducts generated by the deblocking reagent (cleaved protecting group, or other byproduct). The choice of solvent in some instances depends on the choice of deblocking reagent or 5′ protecting group; for example, a deblocking solution comprises a trifluoracetic acid and a solvent comprising toluene. Any combination of deblocking reagents, protecting groups, and solvent is in some instances used with the methods described herein.

For a subsequent cycle of nucleoside incorporation to occur through coupling, a protected 5′ end of the substrate bound growing polynucleotide must be removed so that the primary hydroxyl group can react with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions described herein provide for controlled deblocking conditions limiting undesired depurination reactions. In some instances, the substrate bound polynucleotide is washed after deblocking. In some instances, efficient washing after deblocking contributes to synthesized polynucleotides having a reduced error rate, and/or higher yields.

Exemplary combinations, without limitation, for wash steps and solvents are provided in Table 2, where each different combination of washes is provided by a different number (“No.”) reading from left to right in the table.

TABLE 2
Exemplary solvent wash steps during de novo polynucleotide synthesis.

| | | | | | | | | |
|---|---|---|---|---|---|---|---|---|
| | | | Capping | | | | | |
| | Coupling | | Reaction | Wash 2 | Oxidation | | Deblocking | |
| No. | reaction | Wash 1 | (optional) | (optional) | Reaction | Wash 3 | Reaction | Wash 4 |

| | | | | |
|---|---|---|---|---|
| 1 | Acetone | Acetone | Acetone | Acetone |
| 2 | Acetonitrile | Acetonitrile | Acetone | Acetonitrile |
| 3 | THF | THF | THF | THF |
| 4 | Acetonitrile | Acetonitrile | THF | Acetonitrile |
| 5 | Methyl ethyl | Methyl ethyl | Methyl ethyl | Methyl ethyl |
| | ketone | ketone | ketone | ketone |
| 6 | Acetonitrile | Acetonitrile | Methyl ethyl | Acetonitrile |
| | | | ketone | |

The surface or support-bound polynucleotides may be immobilized through their 3′ end. It should be appreciated that by 3′ end, it is meant the sequence downstream to 5′ end, for example 2, 3, 4, 5, 6, 7, 10, 15, 20 nucleotides or more downstream from the 5′ end, for another example on 3′ half, third, or quarter of the sequence, for yet another example, less than 2, 3, 4, 5, 6, 7, 10, 15, or 20 nucleotides away from the absolute 3′ end and by 5′ end it is meant the sequence upstream to the 3′ end, for example 2, 3, 4, 5, 6, 7, 10, 15, 20 nucleotides or more upstream from 3′ end, for another example on 5′ half, third, or quarter of the sequence, for yet another example, less than 2, 3, 4, 5, 6, 7, 10, 15, or 20 nucleotides away from the absolute 5′ end. For example, a polynucleotide may be immobilized on the support via a nucleotide sequence (e.g., a degenerate binding sequence), a linker or spacer (e.g., a moiety that is not involved in hybridization). In some instances, a linker or spacer comprising nucleosides is homogeneous for a single base. In some instances, a linker or spacer comprising nucleosides is heterogeneous for a single base. In some embodiments, the polynucleotide comprises a spacer or linker to separate the polynucleotide sequence from the support. Useful spacers or linkers include photocleavable linkers, or other traditional chemical linkers. In one embodiment, polynucleotides may be attached to a solid support through a cleavable linkage moiety. For example, the solid support may be functionalized to provide cleavable linkers for covalent attachment to the polynucleotides. The linker moiety may be of six or more atoms in length. Alternatively, the cleavable moiety may be within a polynucleotide and may be introduced during in situ synthesis. A suitable cleavable moiety may be selected to be compatible with the nature of the protecting group of the nucleoside bases, the choice of solid support, and/or the mode of reagent delivery, among others. In an exemplary embodiment, the polynucleotides cleaved from the solid support contain a free 3′-OH end. Alternatively, the free 3′-OH end may also be obtained by chemical or enzymatic treatment, following the cleavage of polynucleotides. In various embodiments, the invention relates to methods and compositions for release of support or surface bound polynucleotides into solution. The cleavable moiety may be removed under conditions which do not degrade the polynucleotides. The linker may be cleaved using two approaches, either simultaneously under the same conditions as the deprotection step or subsequently utilizing a different condition or reagent for linker cleavage after the completion of the deprotection step. Optionally, a capping step is used after any step of linker/spacer synthesis to prevent additional functionalization of unreacted linkers or spacers. In some instances, two or more capping steps are used during synthesis of a polynucleotide spacer.

Provided herein are methods wherein a spacer region is synthesized by iteration of the following steps: 1) extension of a plurality of reactive molecules from a surface by contacting the surface with a base addition solution comprising at least one reactive monomer, wherein the at least one reactive monomer comprising a phosphoramidite nucleoside comprising a 5′ blocking group; 2) capping unreacted 5′ OH groups of the polynucleotide by contacting the surface with a capping solution; 3) washing the surface at least once with a wash solvent; 4) contacting the surface with an oxidizing solution; 5) washing the surface at least once with a wash solvent; 6) capping unreacted 5′ OH groups of the polynucleotide by contacting the surface with a capping solution; 7) removing the 5′ blocking group with a deblocking solution; and 8) washing the surface at least once with a wash solvent. Steps 1-8 are in some instances repeated until a plurality of polynucleotide spacers are synthesized. The wash solvent may be a wash solvent described elsewhere herein.

Provided herein are methods wherein one or more capping steps are used to prevent subsequent reactions with unreacted hydroxyl groups. Capping steps are variously executed before or after any step of polynucleotide synthesis described herein. Often, capping steps are followed with a washing step comprising depositing a wash solvent on the synthesis surface. For example, a capping step is followed by washing with a wash solvent comprising acetone. In some instances, an oxidation step and a capping step are both followed by washing with a wash solvent, such as a wash solvent comprising acetone.

Exemplary combinations, without limitation, for capping steps, wash steps and solvents are provided in Table 3, where each different combination of reaction steps and washes is provided by a different number (“No.”) reading from left to right in the table.

TABLE 3
Exemplary inclusion of capping steps during de novo polynucleotide synthesis.

| | | | | | | | | | | |
|---|---|---|---|---|---|---|---|---|---|---|
| | Coupling | | Capping | Wash 2 | Oxidation | | Capping | | Deblocking | |
| No. | Reaction | Wash 1 | Reaction | (optional) | Reaction | Wash 3 | Reaction | Wash 4 | Reaction | Wash 4 |

| | | | | | |
|---|---|---|---|---|---|
| 1 | Acetone | Acetone | Acetone | Acetone | Acetone |
| 2 | Acetonitrile | Acetonitrile | Acetone | Acetonitrile | Acetonitrile |
| 3 | Acetonitrile | Acetonitrile | Acetone | Acetone | Acetonitrile |
| 4 | THF | THF | THF | THF | THF |
| 5 | Acetonitrile | Acetonitrile | THF | Acetonitrile | Acetonitrile |
| 6 | Acetonitrile | Acetonitrile | THF | THF | Acetonitrile |
| 7 | Methyl | Methyl | Methyl | Methyl | Methyl |
| | ethyl ketone | ethyl ketone | ethyl ketone | ethyl ketone | ethyl ketone |
| 8 | Acetonitrile | Acetonitrile | Methyl | Acetonitrile | Acetonitrile |
| | | | ethyl ketone | | |
| 9 | Acetonitrile | Acetonitrile | Methyl | Methyl ethyl | Acetonitrile |
| | | | ethyl ketone | ketone | |

### Solvents

Provided herein are methods, systems, compositions, and devices for chemical polynucleotide synthesis which comprise the use of solvents, or solvent mixtures. In some instances, a solvent or functional equivalent thereof is used as a wash solvent. In some instances, a solvent or functional equivalent thereof is used as a reaction solvent. Suitable solvents and functional equivalents are selected in some instances based on common inherent properties (density, heat capacity, solubility, polarity, miscibility, boiling point, melting point, viscosity, chemical structure, or other physical property), or performance characteristics (ability to dissolve a specific reagent or salt, reduction in error rate, resistance to degradation by a chemical reagent, heat of mixing with solvent of previous wash or reagent solution, or other performance outcome). Wash solvents are variously used in a wash step after any step in the polynucleotide synthesis, such as after deblocking, elongation, oxidation, capping, or any combination thereof.

Further described herein are exemplary solvents including hydrocarbons (e.g., hexane, decane, benzene, toluene, xylene, isomers thereof, and the like), ethers (e.g., THF, diethyl ether, methyl t-butyl ether, and the like), esters (e.g., methyl acetate, ethyl acetate, tert-butylacetate, etc.), lactones, ketones (e.g., acetone, methyl ethyl ketone, cyclopentanone, and the like), alcohols (e.g., ethanol, butanol, isopropanol, and the like), amides (e.g., DMF, N-methylpyrrolidinone, or other amides), ureas, carbonates (e.g., diethylcarbonate, or other carbonate), carbamates, aldehydes, amines, cyanates, isocyanates, sulfoxides, sulfones, aromatics, heteroaromatics, thiols, phosphoramides, nitriles (e.g., acetonitrile), alkynes, alkenes, alkanes, halogenated solvents (e.g., tetrachloromethane, dichloromethane, chloroform, or other halogenated solvent), silanes, perfluorocarbons (C₂-C₁₈ perfluorinated branched or straight alkanes such as perfluorohexane, perfluoroheptane, perfluorodecane, perfluoro aromatics such as perfluorobenzene, or other perfluorocarbon), supercritical fluids, ionic liquids, compressed gases, and the like. In some instances, wash solvents comprise a nitrile, such as acetonitrile. Solvents optionally comprise additional components such as acids, bases, or salts. In some instances, a solvent used for a non-wash step is the same solvent used for a prior or subsequent wash step.

Further described herein are methods, systems, compositions, and devices comprising solvents such as ethers. Exemplary ethers include diethyl ether, methyl ethyl ether, dibutyl ether, diisopropyl ether, di(n-propyl) ether, di(tert-butyl) ether, cyclopentyl methyl ether, dimethoxymethane, 1,4-dioxane, ethyl tert-butyl ether, 2-(2-methoxyethoxy) ethanol, morpholine, polyethylene glycol, 2-(2-methoxyethoxy) ethanol, tetrahydrofuran, tetrahydropyran, methyl tert-butylether, 2-methyl tetrahydrofuran, glyme, diglyme, and dimethoxyethane. In some instances, a wash solvent comprises tetrahydrofuran. In some instances, ethers comprise C₂-C₈ ethers.

Solvents often comprise ketones and are used with the methods, systems and compositions described herein. Exemplary ketones include acetone, acetophenone, butanone, cyclopentanone, cyclohexanone, cyclobutanone, cyclopropanone, ethyl isopropyl ketone, 2-hexanone, isophorone, mesityl oxide, methyl isobutyl ketone, methyl isopropyl ketone, 3-methyl-2-pentanone, 2-pentanone, and 3-pentanone. In some instances, a wash solvent comprises acetone. In some instances, ketones comprise C₂-C₈ ketones.

Further described herein are solvents that are in some instances “pure” substances wherein the solvent comprises only trace (<0.1%, w/v) of other chemical components (e.g., salts, solvents, other solutes). In some instances, solvents comprise water, for example a solvent comprises (v/v) no more than 0.01%, 0.1%, 0.2%, 0.3%, 0.5%, 1.0%, 2.0%, or no more than 5.0% water. In some instances the purity (w/w) of a solvent is at least 70%, 75%, 80%, 85%, 90%, 95%, 97%, 99%, or at least 99.9% pure. In some instances the purity (w/w) of a solvent is about 70%, 75%, 80%, 85%, 90%, 95%, 97%, 99%, or about 99.9% pure. In some instances, solvents comprise salts or buffers.

Further described herein are wash solvents comprising acetone with a purity of (<0.1%, w/v) of other chemical components (e.g., salts, solvents, other solutes). In some instances, wash solvents comprising acetone comprise water, for example a wash solvent comprising acetone comprises (v/v) no more than 0.01%, 0.1%, 0.2%, 0.3%, 0.5%, 1.0%, 2.0%, or no more than 5.0% water. In some instances the purity (w/w) of a wash solvent comprising acetone is at least 70%, 75%, 80%, 85%, 90%, 95%, 97%, 99%, or at least 99.9% pure. In some instances the purity (w/w) of a wash solvent comprising acetone is about 70%, 75%, 80%, 85%, 90%, 95%, 97%, 99%, or about 99.9% pure. In some instances, wash solvents comprising acetone comprise salts or buffers.

Further described herein are solvents comprising a mixture of two or more solvents. For example, a solvent comprises two solvents mixed at about a 1:1, 2:1, 3:1, 4:1, 5:1, 10:1, 20:1, 50:1, 80:10, 99:1 ratio (v/v). In some instances, a mixture of two solvents comprises at least 1%, 2%, 5%, 10%, 20%, 30%, 40%, 50%, 60%, 70%, 80% 90%, 95%, 97%, or at least 99% of one of the solvents (v/v). In some instances, a mixture of two solvents comprises about 1%, 2%, 5%, 10%, 20%, 30%, 40%, 50%, 60%, 70%, 80% 90%, 95%, 97%, or about 99% of one of the solvents (v/v). Solvents in some instances comprise a mixture of three or more solvents.

Further described herein are solvents comprising a mixture of a ketone and an additional solvent. For example, a solvent comprises a ketone and an additional solvent mixed at about a 1:1, 2:1, 3:1, 4:1, 5:1, 10:1, 20:1, 50:1, 80:10, 99:1 ratio (v/v). In some instances, a mixture of a ketone and an additional solvent comprises at least 1%, 2%, 5%, 10%, 20%, 30%, 40%, 50%, 60%, 70%, 80% 90%, 95%, 97%, or at least 99% a ketone (v/v). In some instances, a mixture of a ketone and an additional solvent comprises about 1%, 2%, 5%, 10%, 20%, 30%, 40%, 50%, 60%, 70%, 80% 90%, 95%, 97%, or about 99% a ketone (v/v). In some instances, the additional solvent is acetonitrile.

Further described herein are solvents comprising a mixture of acetone and an additional solvent. For example, a solvent comprises acetone and an additional solvent mixed at about a 1:1, 2:1, 3:1, 4:1, 5:1, 10:1, 20:1, 50:1, 80:10, 99:1 ratio (v/v). In some instances, a mixture of acetone and an additional solvent comprises at least 1%, 2%, 5%, 10%, 20%, 30%, 40%, 50%, 60%, 70%, 80% 90%, 95%, 97%, or at least 99% acetone (v/v). In some instances, a mixture of acetone and an additional solvent comprises about 1%, 2%, 5%, 10%, 20%, 30%, 40%, 50%, 60%, 70%, 80% 90%, 95%, 97%, or about 99% acetone (v/v). In some instances, the additional solvent is acetonitrile. In some instances, the additional solvent comprises THF. In some instances, the additional solvent is acetonitrile. In some instances, the additional solvent is a solvent mixture of two or more solvents.

Further described herein are various solvent temperatures that are optimized. Solvent temperatures can achieve low error rates and increased uniformity of error rate. In some instances, solvent temperatures increase the solubility of a primary reagent or byproduct from a previous step. For example, the temperature of a solvent for a wash or reagent solution is at least 0, 1, 5, 8, 10, 12, 15, 18, 20, 22, 25, 28, 30, 32, 35, 38, 40, 42, 45, 48, 50, 52, 55, 58, 60, 65, 70, 75, 80, 85, 90, or at least 90 degrees C. In some instances the temperature of a solvent for a wash or reagent solution is no more than 0, 1, 5, 8, 10, 12, 15, 18, 20, 22, 25, 28, 30, 32, 35, 38, 40, 42, 45, 48, 50, 52, 55, 58, 60, 65, 70, 75, 80, 85 or no more than 90 degrees C. In some instances the temperature of a solvent for a wash or reagent solution is about 0, 1, 5, 8, 10, 12, 15, 18, 20, 22, 25, 28, 30, 32, 35, 38, 40, 42, 45, 48, 50, 52, 55, 58, 60, 65, 70, 75, 80, 85 or about 90 degrees C. In some instances the temperature of a solvent for a wash or reagent solution is about 20 degrees C. In some instances the temperature of a solvent for a wash or reagent solution is about 25 degrees C. In some instances the temperature of a solvent for a wash or reagent solution is about 5 to 90, about 5 to 70, about 5 to 50, about 5 to 30, about 10 to 80, about 10 to 70, about 10 to 40, about 15 to 40, about 15 to 30, about 15 to 25, about 20 to 30, about 20 to 45, about 25 to 50, about 30 to 60, about 40 to 90, about 40 to 70, about 40 to 60, or about 60 to 90 degrees C. In some instances the temperature of a solvent for a wash or reagent solution is about 15 to about 25 degrees C. In some instances the temperature of a solvent for a wash or reagent solution is about 20 to 25 degrees C. In some instances the temperature of a solvent for a wash or reagent solution is about 20 to 35 degrees C. In some instances the temperature of a solvent for a wash or reagent solution is about 30 to 45 degrees C. In some instances, the temperature of a solvent is varied between two temperatures over time. In some instances, the temperature is varied across a surface to form a temperature gradient.

Further provided herein are solvents that are selected based on polarity index, or miscibility with other solvents or reagent solutions. For example, a wash solvent is selected that comprises a polarity index that is similar to the polarity of the primary constituent of a previous wash solvent or a reagent solution. Alternately or in combination, the viscosity of a solvent at a given temperature determines an optimum wash solvent. Another variable that is in some instances considered is miscibility. In some cases if two solvents are “miscible”, only one phase will be produced at all proportions for a defined temperature. Polarity index, viscosity, water solubility, and miscibility (non-exhaustive) for various solvents used in the methods described herein are shown in Table 4. In some instances, solvents are combined to further adjust polarity, viscosity, or miscibility.

TABLE 4
Solvent characteristics.

| | Polarity | Viscosity | Solubility in | |
|---|---|---|---|---|
| Solvent Name | Index | (cPoise) | water (% w/w) | Immiscible in |
| acetic acid | 6.2 | 1.25 | 100 | pentane, hexane |
| acetone | 5.1 | 0.32 | 100 | |
| acetonitrile | 5.8 | 0.37 | 100 | pentane, hexane, heptane, cyclohexane |
| benzene | 2.7 | 0.66 | 0.18 | water |
| n-butanol | 4.0 | 0.73 | 0.43 | water |
| butyl acetate | 3.9 | 2.98 | 7.81 | water |
| carbon tetrachloride | 1.6 | 0.97 | 0.08 | water |
| chloroform | 4.1 | 0.57 | 0.815 | water |
| cyclohexane | 0.2 | 1.00 | 0.01 | water, methanol, dimethylsulfoxide, |
| | | | | dimethylformamide |
| 1,2-dichloroethane | 3.5 | 0.79 | 0.81 | water |
| dichloromethane | 3.1 | 0.44 | 1.6 | water |
| dimethylformamide | 6.4 | 0.92 | 100 | heptane, hexane, pentane, diisopropyl ether, |
| | | | | xylene |
| dimethyl sulfoxide | 7.2 | 2.00 | 100 | diethyl ether, heptane, hexane, pentane, |
| | | | | xylene |
| dioxane | 4.8 | 1.54 | 100 | |
| ethanol | 5.2 | 1.20 | 100 | |
| ethyl acetate | 4.4 | 0.45 | 8.7 | water |
| diethyl ether | 2.8 | 0.32 | 6.89 | water |
| heptane | 0.0 | 0.39 | 0.0003 | water, methanol |
| hexane | 0.0 | 0.33 | 0.001 | water, methanol |
| methanol | 5.1 | 0.60 | 100 | pentane |
| methyl tert-butyl ether | 2.5 | 0.27 | 4.8 | water |
| methyl ethyl ketone | 4.7 | 0.45 | 24 | water |
| pentane | 0.0 | 0.23 | 0.004 | water |
| n-propanol | 4.0 | 2.27 | 100 | |
| isopropanol | 3.9 | 2.30 | 100 | |
| diisopropyl ether | 2.2 | 0.37 | — | water |
| tetrahydrofuran | 4.0 | 0.55 | 100 | |
| toluene | 2.4 | 0.59 | 0.051 | water |
| trichloroethylene | 1.0 | 0.57 | 0.11 | water, xylene |
| water | 9.0 | 1.00 | 100 | xylene |
| xylene | 2.5 | 0.51 | 0.018 | water |

Further provided herein are methods, systems, compositions, and devices wherein the viscosity of a solvent for a wash or reagent solution is about 0.01, 0.05, 0.1, 0.2, 0.3, 0.4, 0.5, 0.6, 0.7, 0.8, or about 0.9 cP (centiPoise). In some instances the viscosity of a solvent for a wash or reagent solution is at least 0.01, 0.05, 0.1, 0.2, 0.3, 0.4, 0.5, 0.6, 0.7, 0.8, or at least 0.9 cP. In some instances the viscosity of a solvent for a wash or reagent solution is no more than 0.01, 0.05, 0.1, 0.2, 0.3, 0.4, 0.5, 0.6, 0.7, 0.8, or no more than 0.9 cP. In some instances the viscosity of a solvent for a wash or reagent solution is about 0.01 to 0.8, about 0.01 to 0.6, about 0.1 to 0.5, about 0.1 to 0.4, about 0.2 to 0.3, about 0.2 to 0.4, about 0.2 to 0.6, about 0.4 to 0.9, about 0.4 to 0.7, or about 0.5 to 0.9 cP. In some instances the viscosity of a solvent for a wash or reagent solution is about 0.2 to about 0.4 cP. In some instances the viscosity of a solvent for a wash or reagent solution is about 0.1 to about 0.5 cP. In some instances the viscosity of a solvent for a wash or reagent solution is about 0.3 to about 0.4 cP.

Further provided herein are methods, systems, compositions, and devices wherein the polarity index of a solvent for a wash or reagent solution is about 0, 0.5, 1, 1.5, 2, 2.5, 3, 3.5, 4, 4.5, 5, 5.5, 6, 7, 8, or about 9. In some instances the polarity index of a solvent for a wash or reagent solution is at least 0, 0.5, 1, 1.5, 2, 2.5, 3, 3.5, 4, 4.5, 5, 5.5, 6, 7, 8, or at least 9. In some instances the polarity index of a solvent for a wash or reagent solution is no more than 0, 0.5, 1, 1.5, 2, 2.5, 3, 3.5, 4, 4.5, 5, 5.5, 6, 7, 8, or no more than 9. In some instances the polarity index of a solvent for a wash or reagent solution is about 0 to 9, about 0 to 7, about 1 to 6, about 1 to 5, about 1 to 4, about 1 to 3, about 2 to 6, about 2 to 5, about 2 to 4, about 2 to 3, about 3 to 6, about 3 to 5, about 3 to 4, about 4 to 10, about 4 to 9, about 4 to 8, about 4 to 7, about 5 to 10, about 5 to 9, about 5 to 8, about 5 to 7, or about 5 to 6. In some instances the polarity index of a solvent for a wash or reagent solution is about 4 to about 6. In some instances the polarity index of a solvent for a wash or reagent solution is about 5 to about 6. In some instances the polarity index of a solvent for a wash or reagent solution is about 5 to about 7.

### Error Rates of Large Polynucleotide Libraries

Described herein are several methods are used to evaluate the quality of polynucleotide libraries described herein, including Sanger sequencing, next generation sequencing, or other method for measuring the quality of a polynucleotide library. The quality of a polynucleotide library is may be measured by error rate, species representation, or drop outs frequency. Various metrics for error rates include deletions, insertions, substitutions, mismatches, dropouts, or other discrepancy between a predetermined sequence and a measured polynucleotide sequence. Capping is in some instances used to truncate unreacted molecules remaining after a coupling step, which prevents further growth of the polynucleotide into a full-length polynucleotide. In some instances, error rates are described by the number or percentage of full-length polynucleotides. This percentage is often determined by the efficiency of each coupling step, or an average efficiency of coupling over multiple steps.

Further provided herein are methods for synthesizing polynucleotide libraries wherein the average coupling efficiency is about 80%, 90%, 95%, 96%, 96.2%, 96.5%, 96.8%, 97%, 97.2%, 97.5%, 97.8%, 98%, 98.2%, 98.5%, 99.8%, 99%, 99.1%, 99.2%, 99.3%, 99.4%, 99.5%, 99.6%, 99.7%, 99.8%, 99.9%, 99.95%, or about 99.99%. In some instances the average coupling efficiency is at least 80%, 90%, 95%, 96%, 96.2%, 96.5%, 96.8%, 97%, 97.2%, 97.5%, 97.8%, 98%, 98.2%, 98.5%, 99.8%, 99%, 99.1%, 99.2%, 99.3%, 99.4%, 99.5%, 99.6%, 99.7%, 99.8%, 99.9%, 99.95%, or at least 99.99%. In some instances, such libraries comprise polynucleotides wherein each polynucleotide is at least 50, 100, 150, 200, 250, or at least 300 bases in length.

Further provided herein are error rates for polynucleotide libraries, wherein each polynucleotide comprises at least 50 bases, wherein the percentage of full-length sequences is about 40%, 45%, 50%, 52%, 55%, 58%, 60%, 62%, 65%, 68%, 70%, 72%, 75%, 78%, 80%, 82%, 85%, 88%, or about 90%. In some instances for polynucleotide libraries wherein each polynucleotide comprises at least 50 bases, the percentage of full-length sequences is at least 40%, 45%, 50%, 52%, 55%, 58%, 60%, 62%, 65%, 68%, 70%, 72%, 75%, 78%, 80%, 82%, 85%, 88%, or at least 90%. Further provided herein are error rates for polynucleotide libraries, wherein each polynucleotide comprises at least 100 bases, wherein the percentage of full-length sequences is about 40%, 45%, 50%, 52%, 55%, 58%, 60%, 62%, 65%, 68%, 70%, 72%, 75%, 78%, 80%, 82%, 85%, 88%, or about 90%. In some instances for polynucleotide libraries wherein each polynucleotide comprises at least 100 bases, the percentage of full-length sequences is at least 40%, 45%, 50%, 52%, 55%, 58%, 60%, 62%, 65%, 68%, 70%, 72%, 75%, 78%, 80%, 82%, 85%, 88%, or at least 90%. Further provided herein are error rates for polynucleotide libraries, wherein each polynucleotide comprises at least 150 bases, wherein the percentage of full-length sequences is about 40%, 45%, 50%, 52%, 55%, 58%, 60%, 62%, 65%, 68%, 70%, 72%, 75%, 78%, 80%, 82%, 85%, 88%, or about 90%. In some instances for polynucleotide libraries wherein each polynucleotide comprises at least 150 bases, the percentage of full-length sequences is at least 40%, 45%, 50%, 52%, 55%, 58%, 60%, 62%, 65%, 68%, 70%, 72%, 75%, 78%, 80%, 82%, 85%, 88%, or at least 90%. Further provided herein are error rates for polynucleotide libraries, wherein each polynucleotide comprises at least 200 bases, wherein the percentage of full-length sequences is about 40%, 45%, 50%, 52%, 55%, 58%, 60%, 62%, 65%, 68%, 70%, 72%, 75%, 78%, 80%, 82%, 85%, 88%, or about 90%. In some instances for polynucleotide libraries wherein each polynucleotide comprises at least 200 bases, the percentage of full-length sequences is at least 40%, 45%, 50%, 52%, 55%, 58%, 60%, 62%, 65%, 68%, 70%, 72%, 75%, 78%, 80%, 82%, 85%, 88%, or at least 90%. Further provided herein are error rates for polynucleotide libraries, wherein each polynucleotide comprises at least 300 bases, wherein the percentage of full-length sequences is about 40%, 45%, 50%, 52%, 55%, 58%, 60%, 62%, 65%, 68%, 70%, 72%, 75%, 78%, 80%, 82%, 85%, 88%, or about 90%. In some instances for polynucleotide libraries wherein each polynucleotide comprises at least 300 bases, the percentage of full-length sequences is at least 40%, 45%, 50%, 52%, 55%, 58%, 60%, 62%, 65%, 68%, 70%, 72%, 75%, 78%, 80%, 82%, 85%, 88%, or at least 90%.

Further provided herein are error rates for polynucleotide libraries. In some instances, overall error rates for three different sets of oligonucleotide libraries may be less than 1 error in 900, including 1 error in 1000 bases or less than 1 in 1000 bases—where the error rate is a comparison of synthesized sequences to instructions for synthesis of the polynucleotides. In some instances, one or more quality control steps are used in the workflow. For example, control polynucleotides of various sizes present on the surface are evaluated. Control polynucleotides in some instances are 10, 20, 30, 50, 70, 90, 100, 150, 200, 250, 300, or more than 300 bases in length. Control polynucleotides are of different sizes are often used. In some instances, control polynucleotides are 20 bases in length. In some instances, control polynucleotides are 90 bases in length. In some instances, control polynucleotides are 150 bases in length. In some instances, control polynucleotides are 250 bases in length.

Further described herein are methods for measuring error rates in polynucleotide libraries, such as Next Generation Sequencing. For example, error types (or classes) such as deletions, insertions, or mismatches are measured categorized. Error rates are often expressed for any number of error types. In some instances, mismatches are categorized based on the type of transition, for example base 1 to base 2 (e.g., A to T, T to G, C to G, etc.). In some instances, insertions are categorized based on bases adjacent to the insertion, such as base 1+base 2 (e.g., A+A corresponds to an insertion of A after a base A). In some instances, deletions are categorized based on the type of base deleted, for example Base-(e.g., A-corresponds to a deletion of an A). In some instances, deletion rates are measured as single base deletions, or block deletions. Alternately or in combination, error types are evaluated as a function of base position (relative to the order synthesizes) on the polynucleotide.

Average error rates for polynucleotides synthesized within a library using the systems and methods provided may be less than 1 in 800 bases, less than 1 in 900, 1 in 1000, less than 1 in 1250, less than 1 in 1500, less than 1 in 2000, less than 1 in 3000 or less often. In some instances, average error rates for polynucleotides synthesized within a library using the systems and methods provided are less than 1/1000. In some instances, average error rates for polynucleotides synthesized within a library using the systems and methods provided are less than 1/900. In some instances, average error rates for polynucleotides synthesized within a library using the systems and methods provided are less than 1/800. In some instances, average error rates for polynucleotides synthesized within a library using the systems and methods provided are less than 1/1100. In some instances, aggregate error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for polynucleotides synthesized within a library using the systems and methods provided are less than 1/1000. In some instances, aggregate error rates for polynucleotides synthesized within a library using the systems and methods provided are less than 1/900. In some instances, aggregate error rates for polynucleotides synthesized within a library using the systems and methods provided are less than 1/1100.

In some instances, an error correction enzyme may be used for polynucleotides synthesized within a library using the systems and methods provided can use. In some instances, aggregate error rates for polynucleotides with error correction can be less than 1/1000, 1/1500, 1/2000, 1/3000, 1/4000, 1/5000, 1/6000, 1/7000, 1/8000, 1/9000, 1/10000 or less compared to the predetermined sequences. In some instances, aggregate error rates with error correction for polynucleotides synthesized within a library using the systems and methods provided can be less than 1/1000, 1/1500, 1/2000, 1/3000, 1/4000, 1/5000, 1/6000, 1/7000, 1/8000, 1/9000, 1/10000.

Libraries may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic polynucleotide and gene libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any polynucleotides or genes related with more than 95%, 96%, 97%, 98%, 99%, 99.5%, 99.6%, 99.7%, 99.8%, 99.9% or more similarity or identity have the same sequence. In some instances, the error rate related to a specified locus on a polynucleotide or gene is optimized. Thus, a given locus or a plurality of selected loci of one or more polynucleotides or genes as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more polynucleotides or genes.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99%, or more of the library.

Polynucleotide libraries described herein may be measured in terms of uniformity, a measure of polynucleotide species representation. Uniformity may be measured on both a per cluster and per device basis. In some instances, 99% of the polynucleotides have an abundance that is within about 0.05, 0.1, 0.2, 0.5, 0.7, 1, 1.2, 1.5, 1.7 or about within 2× of the mean abundance. In some instances, 97% of the polynucleotides have an abundance that is within about 0.05, 0.1, 0.2, 0.5, 0.7, 1, 1.2, 1.5, 1.7 or about within 2× of the mean abundance. In some instances, 60%, 75%, 80%, 85%, 90%, 95%, 97%, or 99% of the sequences have an abundance that is within 2× of the mean. In some instances, 60%, 75%, 80%, 85%, 90%, 95%, 97%, or 99% of the sequences have an abundance that is within 1.5× of the mean. In some instances, 60%, 75%, 80%, 85%, 90%, 95%, 97%, or 99% of the sequences have an abundance that is within 1× of the mean.

Use of a wash solvent provided herein may provide for a decreased error rate across a surface. For example, flow effects caused by a solvent moving over a surface in some cases lead to residual reagents left on the surface, or solubility effects that result in a difference in the local concentration of reagents across a surface. Flowing reagent solutions that contact a first portion the surface initially in some instances have different properties (reagent concentration, temperature, viscosity, or other property) than the same reagent solution contacting a second portion of the surface at a later time point; this leads to differences in error rates for the first and second portions of the surface (error gradient). This can lead to incomplete reactions, or in some cases unwanted side reactions in a non-uniform manner across the surface, resulting in errors for the synthesized polynucleotides. Wash solvents provided herein in some cases improve solubility of reagents or reagent byproducts, which decreases or eliminates a reagent concentration gradient across a surface Solid surfaces may be horizontal or vertical, where orientation is relative to the flow of a fluid across the solid surface. In other cases wash solvents provided herein facilitate favorable interactions with residual reagent solution in contact with the surface. In some cases, the difference in error rates across a surface are less than 1/100, 1/500, 1/600, 1/700, 1/800, 1/900, 1/1000, 1/1100, 1/1250, 1/1500, 1/2000, 1/5000, or less than 1/10000. In some cases, the difference in error rates across a surface are at least 1/100,1/500, 1/600, 1/700, 1/800, 1/900, 1/1000, 1/1100, 1/1250, 1/1500, 1/2000, 1/5000, or less than 1/10000. In some cases, the difference in error rates across a surface are about 1/100,1/500, 1/600, 1/700, 1/800, 1/900, 1/1000, 1/1100, 1/1250, 1/1500, 1/2000, 1/5000, or about 1/10000.

The methods, compositions, systems, and devices described herein in some cases comprise a surface such as a flow cell, wherein clusters are distributed between an inlet and an outlet that allow for exchange of reagent solution and washes with loci for polynucleotide synthesis. In some instances, a plurality of inlets and outlets are used. In some instances, the flow cell is horizontal, wherein the solvents and reagent solutions flow perpendicular relative to a vector defined by the force of gravity. In some instances, one or more washes with solvents described herein result in uniform error rates between clusters proximal to an inlet and clusters distal from an inlet. In some instances, the difference in error rate is about 0.0001%, 0.001%, 0.002%, 0.005%, 0.008%, 0.01%, 0.02%, 0.05%, 0.08%, 0.1%, 0.2%, 0.5%, 0.8%, or about 1.0% per micron between an inlet and an outlet. In some instances, the difference in error rate is no more than 0.0001%, 0.001%, 0.002%, 0.005%, 0.008%, 0.01%, 0.02%, 0.05%, 0.08%, 0.1%, 0.2%, 0.5%, 0.8%, or no more than 1.0% per micron between an inlet and an outlet. In some instances, the difference in error rate is at least 0.0001%, 0.001%, 0.002%, 0.005%, 0.008%, 0.01%, 0.02%, 0.05%, 0.08%, 0.1%, 0.2%, 0.5%, 0.8%, or at least 1.0% per micron between an inlet and an outlet.

As fluid flows from the inlet to the outlet, various rates of flow are used herein for reagent solutions or wash solvents. In some instances, the flow rate is about 5, 10, 15, 20, 50, 75, 100, 125, 150, 200, 250, 300, 350, 400, or about 500 uL/s. In some instances, the flow rate is at least 5, 10, 15, 20, 50, 75, 100, 125, 150, 200, 250, 300, 350, 400, or at least 500 uL/s. In some instances, the flow rate is no more than 5, 10, 15, 20, 50, 75, 100, 125, 150, 200, 250, 300, 350, 400, or no more than 500 uL/s. In some instances, the flow rate is about 5 to 500 uL/s, about 10 to 400 uL/s, about 20 to 300 uL/s, about 50 to 500 uL/s, about 50 to 400 uL/s, about 50 to 300 uL/s, about 75 to 300 uL/s, about 100 to 400 uL/s, about 200 to 500 uL/s, or about 40 to 350 uL/s. In some instances, the flow rate is about 40 to 350 uL/s. In some instances, the flow rate is about 75 to 250 uL/s. In some instances, the flow rate is about 50 to 400 uL/s.

### Devices for Polynucleotide Synthesis

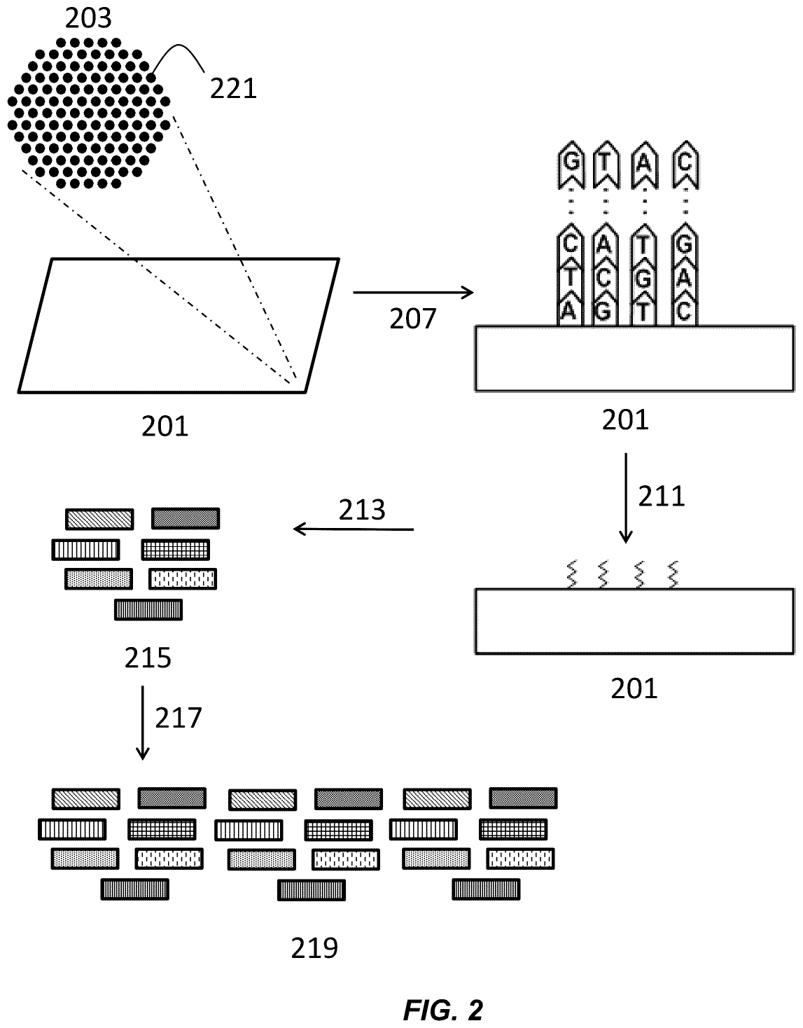

Provided herein are structures having a surface with a plurality of features (loci) for polynucleotide synthesis or extension. Structure may comprise, without limitation, a plate, a film, a tape, a belt, or bead. With regard to structures in the form of an array, each feature in a portion of the structure may comprise a substantially planar feature, a well or a channel. In some instances, the polynucleotides are synthesized on a cluster of loci for polynucleotide extension, released and then subsequently subjected to an amplification reaction, e.g., PCR. An exemplary workflow of synthesis of polynucleotides from a cluster is depicted in FIG. 2. A silicon plate 201 includes multiple clusters 203. Within each cluster are multiple loci 221. Polynucleotides are synthesized 207 de novo on a plate 201 from the cluster 203. Polynucleotides are cleaved 211 and removed 213 from the plate to form a population of released polynucleotides 215. The population of released polynucleotides 215 are then amplified 217 to form a library of amplified polynucleotides 219.

In some instances, a well described herein has a width to depth (or height) ratio of 20 to 0.01, wherein the width is a measurement of the width at the narrowest segment of the well. In some instances, a well described herein has a width to depth (or height) ratio of 20 to 0.05, wherein the width is a measurement of the width at the narrowest segment of the well. In some instances, a well described herein has a width to depth (or height) ratio of 1 to 0.01, wherein the width is a measurement of the width at the narrowest segment of the well. In some instances, a well described herein has a width to depth (or height) ratio of 0.5 to 0.01, wherein the width is a measurement of the width at the narrowest segment of the well. In some instances, a well described herein has a width to depth (or height) ratio of about 0.01, 0.05, 0.1, 0.15, 0.16, 0.2, 0.5, 1, 2, 5, 10 or 20.

In some instances, a well described herein has a diameter to depth (or height) ratio of 20 to 0.01, wherein the diameter is a measurement of the diameter at the narrowest segment of the well. In some instances, a well described herein has a diameter to depth (or height) ratio of 20 to 0.05, wherein the diameter is a measurement of the diameter at the narrowest segment of the well. In some instances, a well described herein has a diameter to depth (or height) ratio of 1 to 0.01, wherein the diameter is a measurement of the diameter at the narrowest segment of the well. In some instances, a well described herein has a diameter to depth (or height) ratio of 0.5 to 0.01, wherein the diameter is a measurement of the diameter at the narrowest segment of the well. In some instances, a well described herein has a diameter to depth (or height) ratio of about 0.01, 0.05, 0.1, 0.15, 0.16, 0.2, 0.5, 1, 2, 5, 10, or 20.

In some instances, a structure described herein comprises a plurality of wells, wherein the height or depth of the well is from about 10 nm to about 10 μm, from about 10 nm to about 1 μm, from about 10 nm to about 500 nm, from about 10 nm to about 100 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, or from about 50 nm to about 100 nm. In some instances, the height of a well is no more than 10 μm, 5 μm, 2 μm, 1 μm, 500 nm, 200 nm, 100 nm, 50 nm, 20 nm, or no more than 10 nm. In some instances, the well height is about 10 nm, 20 nm, 30 nm, 40 nm, 50 nm, 60 nm, 70 nm, 80 nm, 90 nm, 100 nm, 200 nm, 300 nm, 400 nm, 500 nm, 1 μm, 2 μm, 5 μm, 10 μm, or more than 10 μm.

In some instances, a structure described herein comprises a plurality of wells, wherein the width of the well is from about 10 nm to about 10 μm, from about 10 nm to about 1 μm, from about 10 nm to about 500 nm, from about 10 nm to about 100 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, or from about 50 nm to about 100 nm. In some instances, the width of a well is no more than 10 μm, 5 μm, 2 μm, 1 μm, 500 nm, 200 nm, 100 nm, 50 nm, 20 nm, or no more than 10 nm. In some instances, well width is about 10 nm, 20 nm, 30 nm, 40 nm, 50 nm, 60 nm, 70 nm, 80 nm, 90 nm, 100 nm, 200 nm, 300 nm, 400 nm, 500 nm, 1 μm, 2 μm, 5 μm, 10 μm, or more than 10 μm.

In some instances, a structure described herein comprises a plurality of wells, wherein the diameter of the well is from about 10 nm to about 10 μm, from about 10 nm to about 1 μm, from about 10 nm to about 500 nm, from about 10 nm to about 100 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, or from about 50 nm to about 100 nm. In some instances, the diameter of a well is no more than 10 μm, 5 μm, 2 μm, 1 μm, 500 nm, 200 nm, 100 nm, 50 nm, 20 nm, or no more than 10 nm. In some instances, well diameter is about 10 nm, 20 nm, 30 nm, 40 nm, 50 nm, 60 nm, 70 nm, 80 nm, 90 nm, 100 nm, 200 nm, 300 nm, 400 nm, 500 nm, 1 μm, 2 μm, 5 μm, 10 μm, or more than 10 μm.

In some instances, a spot or substantially planar feature described herein has a diameter from about 50 nm to about 1000 nm, from about 50 nm to about 900 nm, from about 50 nm to about 800 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, or from about 50 nm to about 100 nm.

In some instances, a channel described herein has a width to depth (or height) ratio of 20 to 0.01, wherein the channel is a measurement of the width at the narrowest segment of the channel. In some instances, a channel described herein has a width to depth (or height) ratio of 20 to 0.05, wherein the width is a measurement of the width at the narrowest segment of the channel. In some instances, a channel described herein has a width to depth (or height) ratio of 1 to 0.01, wherein the width is a measurement of the width at the narrowest segment of the channel. In some instances, a channel described herein has a width to depth (or height) ratio of 0.5 to 0.01, wherein the width is a measurement of the width at the narrowest segment of the well. In some instances, a channel described herein has a width to depth (or height) ratio of about 0.01, 0.05, 0.1, 0.15, 0.16, 0.2, 0.5, 1, 2, 5, 10 or 20.

In some instances, a channel described herein has a diameter to depth (or height) ratio of 20 to 0.01, wherein the diameter is a measurement of the diameter at the narrowest segment of the channel. In some instances, a channel described herein has a diameter to depth (or height) ratio of 20 to 0.05, wherein the diameter is a measurement of the diameter at the narrowest segment of the channel. In some instances, a channel described herein has a diameter to depth (or height) ratio of 1 to 0.01, wherein the diameter is a measurement of the diameter at the narrowest segment of the channel. In some instances, a channel described herein has a diameter to depth (or height) ratio of 0.5 to 0.01, wherein the diameter is a measurement of the diameter at the narrowest segment of the channel. In some instances, a channel described herein has a diameter to depth (or height) ratio of about 0.01, 0.05, 0.1, 0.15, 0.16, 0.2, 0.5, 1, 2, 5, 10, or 20.

In some instances, a structure described herein comprises a plurality of channels, wherein the height or depth of the channel is from about 10 nm to about 10 μm, from about 10 nm to about 1 μm, from about 10 nm to about 500 nm, from about 10 nm to about 100 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, or from about 50 nm to about 100 nm. In some instances, the height of a channel is no more than 10 μm, 5 μm, 2 μm, 1 μm, 500 nm, 200 nm, 100 nm, 50 nm, 20 nm, or no more than 10 nm. In some instances, channel height is about 10 nm, 20 nm, 30 nm, 40 nm, 50 nm, 60 nm, 70 nm, 80 nm, 90 nm, 100 nm, 200 nm, 300 nm, 400 nm, 500 nm, 1 μm, 2 μm, 5 μm, 10 μm, or more than 10 μm.

In some instances, a structure described herein comprises a plurality of channels, wherein the width of the channel is from about 10 nm to about 10 μm, from about 10 nm to about 1 μm, from about 10 nm to about 500 nm, from about 10 nm to about 100 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, or from about 50 nm to about 100 nm. In some instances, the width of a channel is no more than 10 μm, 5 μm, 2 μm, 1 μm, 500 nm, 200 nm, 100 nm, 50 nm, 20 nm, or no more than 10 nm. In some instances, channel width is about 10 nm, 20 nm, 30 nm, 40 nm, 50 nm, 60 nm, 70 nm, 80 nm, 90 nm, 100 nm, 200 nm, 300 nm, 400 nm, 500 nm, 1 μm, 2 μm, 5 μm, 10 μm, or more than 10 μm.

In some instances, a structure described herein comprises a plurality of channels, wherein the diameter of the channel is from about 10 nm to about 10 μm, from about 10 nm to about 1 μm, from about 10 nm to about 500 nm, from about 10 nm to about 100 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, or from about 50 nm to about 100 nm. In some instances, the diameter of a channel is no more than 10 μm, 5 μm, 2 μm, 1 μm, 500 nm, 200 nm, 100 nm, 50 nm, 20 nm, or no more than 10 nm. In some instances, well diameter is about 10 nm, 20 nm, 30 nm, 40 nm, 50 nm, 60 nm, 70 nm, 80 nm, 90 nm, 100 nm, 200 nm, 300 nm, 400 nm, 500 nm, 1 μm, 2 μm, 5 μm, 10 μm, or more than 10 μm.

In some instances, the width of a feature (e.g., substantially planar feature, well, channel, or other feature supporting polynucleotide synthesis) is from about 10 nm to about 10 μm, from about 100 nm to about 10 μm, from about 200 nm to about 1 μm, from about 50 nm to about 500 nm, from about 50 nm to about 200 μm, or from about 10 nm to about 100 nm, for example, about 10 μm, 5 μm, 2 μm, 1 μm, 500 nm, 200 nm, 100 nm, 50 nm, 20 nm, or 10 nm. In some instances, the width of a feature is no more than about 10 μm, 5 μm, 2 μm, 1 μm, 500 nm, 200 nm, 100 nm, 50 nm or 10 nm. In some instances, the distance between the center of two adjacent features is from about 10 nm to about 10 μm, 20 nm to about 5 μm, from about 50 nm to about 2 nm, from about 100 nm to about 1 μm, from about 200 nm to about 500 nm, from about 200 nm to about 1 μm, from about 200 nm to about 750 nm, or from about 300 nm to about 600 nm, for example, about 500 nm. In some instances, the total width of a feature is about 10 nm, 20 nm, 50 nm, 100 nm, 200 nm, 500 nm, 1 μm, 5 μm, 10 μm, 20 μm, 30 μm, 40 μm, 50 μm, 60 μm, 70 μm, 80 μm, 90 μm, or 100 μm. In some instances, the total width of a feature is about 10 nm to 1 μm, 20 nm to 500 nm, or 50 nm to 100 nm.

### Surfaces for Polynucleotide Synthesis

Provided herein are rigid or flexibles structures for polynucleotide synthesis. In the case of rigid structures, provided herein are devices having a structure (e.g., a plate) for the generation of a library of polynucleotides. An exemplary structure 109 is illustrated in FIG. 1D, wherein the structure 109 has about the same size dimensions as a standard 96 well plate: 140 mm by 90 mm. The structure 109 comprises clusters grouped in 24 regions or sub-fields 115, each sub-field 115 comprising an array of 256 clusters 110. An expanded view of an exemplary sub-field 115 is shown in FIG. 1E. In the expanded view of four clusters (FIG. 1E), a single cluster 110, has a Y axis cluster pitch (distance from center to center of adjacent clusters) of 1079.210 μm or 1142.694 μm, and an X axis cluster pitch of 1125 μm. An illustrative cluster 110 is depicted in FIG. 1F, where the Y axis loci pitch (distance from center to center of adjacent loci) is 63.483 μm, and an X axis loci pitch is 75 μm. The locus width at the longest part, e.g., diameter for a circular locus, is 50 μm and the distance between loci is 24 μm. The number of loci 111 in the exemplary cluster in FIG. 1F is 121. The loci may be flat, wells, or channels.

In the case of flexible structures, provided herein are devices wherein the flexible structure comprises a continuous loop 105 wrapped around one or more fixed structures, e.g., a pair of rollers 106 or a non-continuous flexible structure 107 wrapped around separate fixed structures, e.g., a pair reels 108. See FIGS. 1B-1C. In some instances, the structures comprise multiple regions for polynucleotide synthesis.

In some instances, each feature supports the synthesis of a population of polynucleotides having a different sequence than a population of polynucleotides grown on another feature. Provided herein are surfaces which comprise at least 10, 100, 256, 500, 1,000, 2,000, 3,000, 4,000, 5,000, 6,000, 7,000, 8,000, 9,000, 10,000, 11,000, 12,000, 13,000, 14,000, 15,000, 20,000, 30,000, 40,000, 50,000 or more clusters. Provided herein are surfaces which comprise more than 2,000; 5,000; 10,000; 20,000; 30,000; 50,000; 100,000; 200,000; 300,000; 400,000; 500,000; 600,000; 700,000; 800,000; 900,000; 1,000,000; 5,000,000; or 10,000,000 or more distinct features. In some instances, each cluster includes 1, 2, 3, 4, 5, 6, 7, 8, 9, 10, 20, 30, 40, 50, 60, 70, 80, 90, 100, 120, 130, 150, 200, 500 or more features. In some instances, each cluster includes 50 to 500, 50 to 200, 50 to 150, or 100 to 150 features. In some instances, each cluster includes 100 to 150 features. In exemplary arrangements, each cluster includes 109, 121, 130 or 137 features. In some instances, each structure within a feature supports the synthesis of a population of polynucleotides having a different sequence than a population of polynucleotides grown on another structure, within the same feature. Provided herein are features which in some instances each comprise at least 1; 2; 5; 10; 20; 50; 100; 200, 500, 1,000, 2,000, 5,000, 10,000, 20,000 or more than 200,000 distinct structures. In some instances, each feature comprises about 10 to about 500, about 50 to about 250, about 10 to about 1000, or about 1 to about 50 structures.

In some instances, the distance from the centers of two adjacent loci within a cluster is from about 10 μm to about 500 μm, from about 10 μm to about 200 μm, or from about 10 μm to about 100 μm. In some instances, the distance from two centers of adjacent loci is greater than about 10 μm, 20 μm, 30 μm, 40 μm, 50 μm, 60 μm, 70 μm, 80 μm, 90 μm or 100 μm. In some instances, the distance from the centers of two adjacent loci is less than about 200 μm, 150 μm, 100 μm, 80 μm, 70 μm, 60 μm, 50 μm, 40 μm, 30 μm, 20 μm or 10 μm. In some instances, each locus has a width of about 0.5 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm or 100 μm. In some instances, each locus has a width of about 0.5 μm to 100 μm, about 0.5 μm to 50 μm, about 10 μm to 75 μm, or about 0.5 μm to 50 μm.

In some instances, the density of clusters within a device is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a device comprises from about 1 cluster per 10 mm² to about 10 clusters per 1 mm². In some instances, the distance from the centers of two adjacent clusters is less than about 50 μm, 100 μm, 200 μm, 500 μm, 1000 μm, or 2000 μm or 5000 μm. In some instances, the distance from the centers of two adjacent clusters is from about 50 μm and about 100 μm, from about 50 μm and about 200 μm, from about 50 μm and about 300 μm, from about 50 μm and about 500 μm, and from about 100 μm to about 2000 μm. In some instances, the distance from the centers of two adjacent clusters is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, each cluster has a diameter or width along one dimension of about 0.5 to 2 mm, about 0.5 to 1 mm, or about 1 to 2 mm. In some instances, each cluster has a diameter or width along one dimension of about 0.5, 0.6, 0.7, 0.8, 0.9, 1, 1.1, 1.2, 1.3, 1.4, 1.5, 1.6, 1.7, 1.8, 1.9 or 2 mm. In some instances, each cluster has an interior diameter or width along one dimension of about 0.5, 0.6, 0.7, 0.8, 0.9, 1, 1.1, 1.15, 1.2, 1.3, 1.4, 1.5, 1.6, 1.7, 1.8, 1.9 or 2 mm.

Provided herein are features having a width at the longest segment of 10 nm to 1 μm. In some instances, the features have a width at the longest segment of about 10, 20, 30, 35, 40, 45, 50, 55 or 60 nm. In some instances, the features are channels having multiple segments, wherein each segment has a center to center distance apart of 5 to 50 nm. In some instances, the center to center distance apart for each segment is about 5, 10, 15, 20 or 25 nm.

The number of distinct polynucleotides synthesized on the surface of a structure described herein is often dependent on the number of distinct features available in the substrate. In some instances, the density of features within a cluster of a substrate is at least or about 1 feature per mm², 10 features per mm², 25 features per mm², 50 features per mm², 65 features per mm², 75 features per mm², 100 features per mm², 130 features per mm², 150 features per mm², 175 features per mm², 200 features per mm², 300 features per mm², 400 features per mm², 500 features per mm², 1,000 features per mm², 2,000 features per mm², 5,000 features per mm². 10,000 features per mm². 100,000 features per mm². 1,000,000 features per mm² or more than 1,000,000 features per mm². In some instances, a substrate comprises from about 10 features per mm² to about 500 features per mm², from about 25 features per mm² to about 400 features per mm², from about 50 features per mm² to about 500 features per mm², from about 100 features per mm² to about 500 features per mm², from about 150 features per mm² to about 500 features per mm², from about 10 features per mm² to about 250 features per mm², from about 50 features per mm² to about 250 features per mm², from about 10 features per mm² to about 200 features per mm², or from about 50 features per mm² to about 200 features per mm².

In some instances, the density of features within a cluster of a substrate is at least or about 1 feature per μm², 10 features per μm², 25 features per μm², 50 features per μm², 65 features per μm², 75 features per μm², 100 features per μm², 130 features per μm², 150 features per μm², 175 features per μm², 200 features per μm², 300 features per μm², 400 features per μm², 500 features per μm², 1,000 features per μm², 2,000 features per μm², 5,000 features per μm². 10,000 features per μm². 100,000 features per μm². 1,000,000 features per μm² or more than 1,000,000 features per μm². In some instances, a substrate comprises from about 10 features per μm² to about 500 features per μm², from about 25 features per μm² to about 400 features per μm², from about 50 features per μm² to about 500 features per μm², from about 100 features per μm² to about 500 features per μm², from about 150 features per μm² to about 500 features per μm², from about 10 features per μm² to about 250 features per μm², from about 50 features per μm² to about 250 features per μm², from about 10 features per μm² to about 200 features per μm², or from about 50 features per μm² to about 200 features per μm². In some instances, the distance between the centers of two adjacent features within a cluster is from about 10 μm to about 500 μm, from about 10 μm to about 200 μm, or from about 10 μm to about 100 μm. In some instances, the distance between two centers of adjacent features is greater than about 10 μm, 20 μm, 30 μm, 40 μm, 50 μm, 60 μm, 70 μm, 80 μm, 90 μm or 100 μm. In some instances, the distance between the centers of two adjacent features is less than about 200 μm, 150 μm, 100 μm, 80 μm, 70 μm, 60 μm, 50 μm, 40 μm, 30 μm, 20 μm or 10 μm. In some instances, the distance between the centers of two adjacent features within a cluster is from about 10 nm to about 1000 nm, from about 10 nm to about 500 nm, 10 nm to about 200 nm, or from about 10 nm to about 100 nm. In some instances, the distance between two centers of adjacent features is greater than about 10 nm, 20 nm, 30 nm, 40 nm, 50 nm, 60 nm, 70 nm, 80 nm, 90 nm or 100 nm. In some instances, the distance between the centers of two adjacent features is less than about 500 nm, 200 nm, 150 nm, 100 nm, 80 nm, 70 nm, 60 nm, 50 nm, 40 nm, 30 nm, 20 nm or 10 nm. In some instances, each square meter of a structure described herein allows for at least about 10⁷, 10⁸, 10⁹, 10¹⁰, 10¹¹ or at least about 10¹² features, where each feature supports one polynucleotide. In some instances, each square meter of a structure described herein allows for at least about 10⁷, 10⁸, 10⁹, 10¹⁰, 10¹¹ or at least about 10¹² features, where each feature supports a plurality of different polynucleotides. In some instances, 10⁹ polynucleotides are supported on less than about 6, 5, 4, 3, 2 or 1 m² of a structure described herein.

In some instances, a structure described herein provides support for the synthesis of more than 2,000; 5,000; 10,000; 20,000; 30,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000; 100,000,000 or more non-identical polynucleotides. In some instances, the structure provides support for the synthesis of more than 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000; 100,000,000 or more polynucleotides encoding for distinct sequences. In some instances, at least a portion of the polynucleotides have an identical sequence or are configured to be synthesized with an identical sequence. In some instances, the structure provides a surface environment for the growth of polynucleotides having at least about 50, 60, 70, 75, 80, 85, 90, 95, 100, 110, 120, 130, 140, 150, 160, 175, 200, 225, 250, 275, 300, 325, 350, 375, 400, 425, 450, 475, 500, 1,000, 2,000 bases or more than 2,000 bases. In some instances, the structure provides a surface environment for the growth of polynucleotides each having at least about 50, 60, 70, 75, 80, 85, 90, 95, 100, 110, 120, 130, 140, 150, 160, 175, 200, 225, 250, 275, 300, 325, 350, 375, 400, 425, 450, 475, 500, 1,000, 2,000 bases or more than 2,000 bases. In some instances, the structure provides a surface environment for the growth of polynucleotides having between 50 and 2,000, bases, 50 and 1,000, 50 and 500, 50 and 250, or between 100 and 1,000, 100 and 500, or between 100 and 300 bases.

In some instances, polynucleotides are synthesized on distinct features of a structure, wherein each feature supports the synthesis of a population of polynucleotides. In some instances, each feature supports the synthesis of a population of polynucleotides having a different sequence than a population of polynucleotides grown on another locus. In some instances, the features of a structure are located within a plurality of clusters. In some instances, a structure comprises at least 10, 500, 1,000, 2,000, 3,000, 4,000, 5,000, 6,000, 7,000, 8,000, 9,000, 10,000, 11,000, 12,000, 13,000, 14,000, 15,000, 20,000, 30,000, 40,000, 50,000 or more clusters. In some instances, a structure comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct features. In some instances, each cluster includes 1, 2, 3, 4, 5, 6, 7, 8, 9, 10, 20, 30, 40, 50, 60, 70, 80, 90, 100, 120, 130, 150 or more features (loci). In some instances, each cluster includes 50 to 500, 100 to 150, or 100 to 200 features. In some instances, each cluster includes 109, 121, 130 or 137 features. In some instances, each cluster includes 5, 6, 7, 8, 9, 10, 11 or 12 features. In some instances, polynucleotides from distinct features within one cluster have sequences that, when assembled, encode for a contiguous longer polynucleotide of a predetermined sequence.

In some instances, a structure described herein is about the size of a standard 96 well plate, for example between about 100 and 200 mm by between about 50 and 150 mm. In some instances, a structure described herein has a diameter less than or equal to about 1000 mm, 500 mm, 450 mm, 400 mm, 300 mm, 250 nm, 200 mm, 150 mm, 100 mm or 50 mm. In some instances, the diameter of a substrate is between about 25 mm and 1000 mm, between about 25 mm and about 800 mm, between about 25 mm and about 600 mm, between about 25 mm and about 500 mm, between about 25 mm and about 400 mm, between about 25 mm and about 300 mm, or between about 25 mm and about 200. Non-limiting examples of substrate size include about 300 mm, 200 mm, 150 mm, 130 mm, 100 mm, 76 mm, 51 mm and 25 mm. In some instances, a substrate has a planar surface area of at least about 100 mm²; 200 mm²; 500 mm²; 1,000 mm²; 2,000 mm²; 5,000 mm²; 10,000 mm²; 12,000 mm²; 15,000 mm²; 20,000 mm²; 30,000 mm²; 40,000 mm²; 50,000 mm² or more. In some instances, a substrate has a thickness between about 50 mm and about 2000 mm, between about 50 mm and about 1000 mm, between about 100 mm and about 1000 mm, between about 200 mm and about 1000 mm, or between about 250 mm and about 1000 mm. Non-limiting examples of thickness include 275 mm, 375 mm, 525 mm, 625 mm, 675 mm, 725 mm, 775 mm and 925 mm. In some instances, the thickness of the substrate varies with diameter and depends on the composition of the substrate. For example, a structure comprising materials other than silicon may have a different thickness than a silicon structure of the same diameter. Structure thickness may be determined by the mechanical strength of the material used and the structure must be thick enough to support its own weight without cracking during handling. In some instances, a structure is more than about 1, 2, 3, 4, 5, 10, 15, 30, 40, 50 feet in any one dimension.

### Surface Architecture

Provided herein are devices comprising raised and/or lowered features. One benefit of having such features is an increase in surface area to support polynucleotide synthesis. In some instances, a device having raised and/or lowered features is referred to as a three-dimensional substrate. In some instances, a three-dimensional device comprises one or more channels. In some instances, one or more loci comprise a channel. In some instances, the channels are accessible to reagent deposition via a deposition device such as a polynucleotide synthesizer. In some instances, reagents and/or fluids collect in a larger well in fluid communication one or more channels. For example, a device comprises a plurality of channels corresponding to a plurality of loci with a cluster, and the plurality of channels are in fluid communication with one well of the cluster. In some methods, a library of polynucleotides is synthesized in a plurality of loci of a cluster.

In some instances, the structure is configured to allow for controlled flow and mass transfer paths for polynucleotide synthesis on a surface. In some instances, the configuration of a device allows for the controlled and even distribution of mass transfer paths, chemical exposure times, and/or wash efficacy during polynucleotide synthesis. In some instances, the configuration of a device allows for increased sweep efficiency, for example by providing sufficient volume for a growing a polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are methods to synthesize an amount of nucleic acid, such as RNA or DNA, of 1 fM, 5 fM, 10 fM, 25 fM, 50 fM, 75 fM, 100 fM, 200 fM, 300 fM, 400 fM, 500 fM, 600 fM, 700 fM, 800 fM, 900 fM, 1 pM, 5 pM, 10 pM, 25 pM, 50 pM, 75 pM, 100 pM, 200 pM, 300 pM, 400 pM, 500 pM, 600 pM, 700 pM, 800 pM, 900 pM, or more. In some instances, a polynucleotide library may span the length of about 1%, 2%, 3%, 4%, 5%, 10%, 15%, 20%, 30%, 40%, 50%, 60%, 70%, 80%, 90%, 95%, or 100% of a gene. A gene may be varied up to about 1%, 2%, 3%, 4%, 5%, 10%, 15%, 20%, 30%, 40%, 50%, 60%, 70%, 80%, 85%, 90%, 95%, or 100%.

Non-identical polynucleotides may collectively encode a sequence for at least 1%, 2%, 3%, 4%, 5%, 10%, 15%, 20%, 30%, 40%, 50%, 60%, 70%, 80%, 85%, 90%, 95%, or 100% of a gene. In some instances, a polynucleotide may encode a sequence of 50%, 60%, 70%, 80%, 85%, 90%, 95%, or more of a gene. In some instances, a polynucleotide may encode a sequence of 80%, 85%, 90%, 95%, or more of a gene.

In some instances, segregation is achieved by physical structure. In some instances, segregation is achieved by differential functionalization of the surface generating active and passive regions for polynucleotide synthesis. Differential functionalization is also be achieved by alternating the hydrophobicity across the device surface, thereby creating water contact angle effects that cause beading or wetting of the deposited reagents. Employing larger structures can decrease splashing and cross-contamination of distinct polynucleotide synthesis locations with reagents of the neighboring spots. In some instances, a device, such as a polynucleotide synthesizer, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000; 1:3,000; 1:5,000; or 1:10,000). In some instances, a device comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a device may have the same or different width, height, and/or volume as another well of the substrate. A channel of a device may have the same or different width, height, and/or volume as another channel of the substrate. In some instances, the width of a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a well comprising a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a cluster is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a cluster is from about 1.0 and 1.3 mm. In some instances, the width of a cluster is about 1.150 mm. In some instances, the width of a well is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a well is from about 1.0 and 1.3 mm. In some instances, the width of a well is about 1.150 mm. In some instances, the width of a cluster is about 0.08 mm. In some instances, the width of a well is about 0.08 mm. The width of a cluster may refer to clusters within a two-dimensional or three-dimensional substrate.

In some instances, the height of a well is from about 20 μm to about 1000 μm, from about 50 μm to about 1000 μm, from about 100 μm to about 1000 μm, from about 200 μm to about 1000 μm, from about 300 μm to about 1000 μm, from about 400 μm to about 1000 μm, or from about 500 μm to about 1000 μm. In some instances, the height of a well is less than about 1000 μm, less than about 900 μm, less than about 800 μm, less than about 700 μm, or less than about 600 μm.

In some instances, a device comprises a plurality of channels corresponding to a plurality of loci within a cluster, wherein the height or depth of a channel is from about 5 μm to about 500 μm, from about 5 μm to about 400 μm, from about 5 μm to about 300 μm, from about 5 μm to about 200 μm, from about 5 μm to about 100 μm, from about 5 μm to about 50 μm, or from about 10 μm to about 50 μm. In some instances, the height of a channel is less than 100 μm, less than 80 μm, less than 60 μm, less than 40 μm or less than 20 μm.

In some instances, the diameter of a channel, locus (e.g., in a substantially planar substrate) or both channel and locus (e.g., in a three-dimensional device wherein a locus corresponds to a channel) is from about 1 μm to about 1000 μm, from about 1 μm to about 500 μm, from about 1 μm to about 200 μm, from about 1 μm to about 100 μm, from about 5 μm to about 100 μm, or from about 10 μm to about 100 μm, for example, about 90 μm, 80 μm, 70 μm, 60 μm, 50 μm, 40 μm, 30 μm, 20 μm or 10 μm. In some instances, the diameter of a channel, locus, or both channel and locus is less than about 100 μm, 90 μm, 80 μm, 70 μm, 60 μm, 50 μm, 40 μm, 30 μm, 20 μm or 10 μm. In some instances, the distance from the center of two adjacent channels, loci, or channels and loci is from about 1 μm to about 500 μm, from about 1 μm to about 200 μm, from about 1 μm to about 100 μm, from about 5 μm to about 200 μm, from about 5 μm to about 100 μm, from about 5 μm to about 50 μm, or from about 5 μm to about 30 μm, for example, about 20 μm.

### Surface Materials

Provided herein are devices comprising a surface, wherein the surface is modified to support polynucleotide synthesis at predetermined locations and with a resulting low error rate, a low dropout rate, a high yield, and a high oligo representation. In some instances, surfaces of a device for polynucleotide synthesis provided herein are fabricated from a variety of materials capable of modification to support a de novo polynucleotide synthesis reaction. In some instances, the devices are sufficiently conductive, e.g., are able to form uniform electric fields across all or a portion of the device. A device described herein may comprise a flexible material. Exemplary flexible materials include, without limitation, modified nylon, unmodified nylon, nitrocellulose, and polypropylene. A device described herein may comprise a rigid material. Exemplary rigid materials include, without limitation, glass, fuse silica, silicon, silicon dioxide, silicon nitride, metal nitride, metal silicide, metal carbide, metal oxide, plastics (for example, polytetrafluoroethylene, polypropylene, polystyrene, polycarbonate, and blends thereof), and metals (for example, gold, platinum). In some instances, metal oxides include TiO₂, Ta₂O₅, Nb₂O₅, Al₂O₃, BaO, Y₂O₃, HfO₂, SrO or other metal oxide known in the art. In some instances, metal carbides include TIC, WC, ThC₂, ThC, VC, W₂C, ZrC, HfC, NbC, TaC, Ta₂C, or other metal carbide known in the art. In some instances, metal nitrides include GaN, InN, BN, Be₃N₂, Cr₂N, MON, Si₃N₄, TaN, Th₂N₂, VN, ZrN, TIN, HIN, NbC, WN, TaN, or other metal nitride known in the art. Devices disclosed herein are in some instances fabricated from a material comprising silicon, polystyrene, agarose, dextran, cellulosic polymers, polyacrylamides, polydimethylsiloxane (PDMS), glass, or any combination thereof. In some instances, a device disclosed herein is manufactured with a combination of materials listed herein or any other suitable material known in the art.

A listing of tensile strengths for exemplary materials described herein is provides as follows: nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. A listing of Young's modulus for stiffness of exemplary materials described herein is provides as follows: nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load.

In some instances, a device disclosed herein comprises a silicon dioxide base and a surface layer of silicon oxide. Alternatively, the device may have a base of silicon oxide. Surface of the device provided here may be textured, resulting in an increase overall surface area for polynucleotide synthesis. Devices described herein may comprise at least 5%, 10%, 25%, 50%, 80%, 90%, 95%, or 99% silicon. A device disclosed herein may be fabricated from a silicon on insulator (SOI) wafer.

### Surface Modifications

In various instances, surface modifications are employed for the chemical and/or physical alteration of a surface by an additive or subtractive process to change one or more chemical and/or physical properties of a device surface or a selected site or region of a device surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some instances, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some instances, the adhesion promoter is a chemical with a high surface energy. In some instances, a second chemical layer is deposited on a surface of a substrate. In some instances, the second chemical layer has a low surface energy. In some instances, surface energy of a chemical layer coated on a surface supports localization of droplets on the surface. Depending on the patterning arrangement selected, the proximity of loci and/or area of fluid contact at the loci are alterable.

In some instances, a device surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a device surface is modified with one or more different layers of compounds. Such modification layers of interest include, without limitation, inorganic and organic layers such as metals, metal oxides, polymers, small organic molecules and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyethylencamines, polyarylene sulfides, polysiloxanes, polyimides, polyacetates, and any other suitable compounds described herein or otherwise known in the art. In some instances, polymers are heteropolymeric. In some instances, polymers are homopolymeric. In some instances, polymers comprise functional moieties or are conjugated.

In some instances, resolved loci of a device are functionalized with one or more moieties that increase and/or decrease surface energy. In some instances, a moiety is chemically inert. In some instances, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for device functionalization may comprise: (a) providing a device having a surface that comprises silicon dioxide; and (b) silanizing the surface using, a suitable silanizing agent described herein or otherwise known in the art, for example, an organofunctional alkoxysilane molecule.

In some instances, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some instances, a device surface comprises functionalized with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluorocthylene. Other methods and functionalizing agents are described in U.S. Pat. No. 5,474,796, which is herein incorporated by reference in its entirety.

In some instances, a device surface is functionalized by contact with a derivatizing composition that contains a mixture of silanes, under reaction conditions effective to couple the silanes to the device surface, typically via reactive hydrophilic moieties present on the device surface. Silanization generally covers a surface through self-assembly with organofunctional alkoxysilane molecules.

A variety of siloxane functionalizing reagents can further be used as currently known in the art, e.g., for lowering or increasing surface energy. The organofunctional alkoxysilanes can be classified according to their organic functions.

Provided herein are devices that may contain patterning of agents capable of coupling to a nucleoside. In some instances, a device may be coated with an active agent. In some instances, a device may be coated with a passive agent. Exemplary active agents for inclusion in coating materials described herein includes, without limitation, N-(3-triethoxysilylpropyl)-4-hydroxybutyramide (HAPS), 11-acetoxyundecyltricthoxysilane, n-decyltricthoxysilane, (3-aminopropyl)trimethoxysilane, (3-aminopropyl)tricthoxysilanc, 3-glycidoxypropyltrimethoxysilane (GOPS), 3-iodo-propyltrimethoxysilane, butyl-aldehyde-trimethoxysilane, dimeric secondary aminoalkyl siloxanes, (3-aminopropyl)-dicthoxy-methylsilane, (3-aminopropyl)-dimethyl-ethoxysilane, and (3-aminopropyl)-trimethoxysilane, (3-glycidoxypropyl)-dimethyl-cthoxysilane, glycidoxy-trimethoxysilanc, (3-mercaptopropyl)-trimcthoxysilane, 3-4 epoxycyclohexyl-ethyltrimethoxysilane, and (3-mercaptopropyl)-methyl-dimethoxysilane, allyl trichlorochlorosilane, 7-oct-1-enyl trichlorochlorosilane, or bis(3-trimethoxysilylpropyl)amine.

Exemplary passive agents for inclusion in a coating material described herein includes, without limitation, perfluorooctyltrichlorosilane; tridecafluoro-1,1,2,2-tetrahydrooctyl)trichlorosilane; 1H, 1H, 2H, 2H-fluorooctyltricthoxysilane (FOS); trichloro(1H, 1H, 2H, 2H-perfluorooctyl) silane; tert-butyl-[5-fluoro-4-(4,4,5,5-tetramethyl-1,3,2-dioxaborolan-2-yl) indol-1-yl]-dimethyl-silane; CYTOP™; Fluorinert™; perfluoroctyltrichlorosilane (PFOTCS); perfluorooctyldimethylchlorosilane (PFODCS); perfluorodecyltriethoxysilane (PFDTES); pentafluorophenyl-dimethylpropylchloro-silanc (PFPTES); perfluorooctyltricthoxysilanc; perfluorooctyltrimethoxysilane; octylchlorosilane; dimethylchloro-octodecyl-silanc; methyldichloro-octodecyl-silane; trichloro-octodecyl-silane; trimethyl-octodecyl-silane; triethyl-octodecyl-silane; or octadecyltrichlorosilanc.

In some instances, a functionalization agent comprises a hydrocarbon silane such as octadecyltrichlorosilane. In some instances, the functionalizing agent comprises 11-acctoxyundecyltricthoxysilane, n-decyltricthoxysilane, (3-aminopropyl)trimethoxysilane, (3-aminopropyl)tricthoxysilane, glycidyloxypropyl/trimethoxysilane and N-(3-tricthoxysilylpropyl)-4-hydroxybutyramide.

Provided herein are devices for polynucleotide synthesis comprising a structure fabricated from any one or more of a variety of materials. In certain instances, the materials from which the substrates/solid supports comprise are fabricated to exhibit a low level of polynucleotide binding. In some situations, materials that are transparent to visible and/or UV light can be employed. Materials that are sufficiently conductive (conductors), e.g., those that can form uniform electric fields across all or a portion of the substrates/solids support described herein, can be utilized. In some instances, such materials may be connected to an electric ground. In some instances, the substrate or solid support can be heat conductive or insulated. The materials can be chemical resistant and heat resistant to support chemical or biochemical reactions such as a series of polynucleotide synthesis reactions.

In some instances, conductive or semiconductive materials (semiconductors) include but are not limited to one or more of titanium silicon nitride, titanium nitride, tungsten nitride, tantulum nitride, tantulum silicon nitride, titanium, platinum silicide, or other conductive materials. In instances materials include but are not limited to one or more of aluminum carbides, carbides, nitrides, oxides, silicides, siliconitrides, phosphides, or other non-metal or metalloids used as components of conductive materials. In some instances, exemplary materials comprise (non-limiting) one or more of the elements of tungsten, cobalt, iridium, molybdenum, nickel, platinum, rhenium, ruthenium, tantulum, titanium, or other, metals used as components of conductive materials. In some instances, materials comprise mixtures of metals, non-metals, or metalloids. In some instances, dopants are added to the semiconductive material. Dopants include but are not limited to phosphorus, antimony, arsenic, boron, aluminum, indium, or other element consistent with the specification. Conductors, semiconductors, or insulators may be manufactured with a combination of materials listed herein or any other suitable material known in the art.

For rigid materials, specific materials of interest include: glass; fused silica; silicon, plastics (for example polytetrafluoroethylene, polypropylene, polystyrene, polycarbonate, and blends thereof, and the like); metals (for example, gold, platinum, and the like). The structure can be fabricated from a material selected from the group consisting of silicon, polystyrene, agarose, dextran, cellulosic polymers, polyacrylamides, polydimethylsiloxane (PDMS), and glass. The substrates/solid supports, microstructures, reactors, or other polynucleotide synthesis structure therein may be manufactured with a combination of materials listed herein or any other suitable material known in the art.

Exemplary flexible materials for structures described herein include, without limitation, nylon (unmodified nylon, modified nylon, clear nylon), nitrocellulose, polypropylene, polycarbonate, polyethylene, polyurethane, polystyrene, acetal, acrylic, acrylonitrile, butadiene styrene (ABS), polyester films such as polyethylene terephthalate, polymethyl methacrylate or other acrylics, polyvinyl chloride or other vinyl resin, transparent PVC foil, transparent foil for printers, Poly(methyl methacrylate) (PMMA), methacrylate copolymers, styrenic polymers, high refractive index polymers, fluorine-containing polymers, polyethersulfone, polyimides containing an alicyclic structure, rubber, fabric, metal foils, and any combination thereof. Various plasticizers and modifiers may be used with polymeric substrate materials to achieve selected flexibility characteristics.

Flexible structures described herein may comprise a plastic material. In some instances, the structure comprises a thermoplastic material. Non-limiting examples of thermoplastic materials include acrylic, acrylonitrile butadiene styrene, nylon, polylactic acid, polybenzimidazole, polycarbonate, polyether sulfone, polyetherether ketone, polyetherimide, polyethylene, polyphenylene oxide, polyphenylene sulfide, polypropylene, polystyrene, polyvinyl chloride, and polytetrafluorocthylene. In some instances, the substrate comprises a thermoplastic material in the polyaryletherketone (PEAK) family. Non-limiting examples of PEAK thermoplastics include polyetherketone (PEK), polyetherketoneketone (PEKK), poly(ether ketone ketonc) (PEEKK), polyether ether ketone (PEEK), and polyetherketoneetherketoneketone (PEKEKK). In some instances, the structure comprises a thermoplastic material compatible with toluene. In some instances, the flexibility of the plastic material is increased by the addition of a plasticizer. An example of a plasticizer is an ester-based plasticizer, such as phthalate. Phthalate plasticizers include bis(2-ethylhexyl) phthalate (DEHP), diisononly phthalate (DINP), di-n-butyl phthalate (DnBP, DBP), butyl benzyl phthalate (BBzP), diisodecyl phthalate (DIDP), dioctyl phthalate (DOP, DnOP), diisooctyl phthalate (DIOP), diethyl phthalate (DEP), diisobutyl phthalate (DIBP), and di-n-hexyl phthalate. In some instances, modification of the thermoplastic polymer through copolymerization or through the addition of non-reactive side chains to monomers before polymerization also increases flexibility.

Provided herein are flexible structures which may further comprise a fluoroelastomer. Materials having about 80% fluoroclastomers are designated as FKMs. Fluoroclastomers include perfluoro-elastomers (FFKMs) and tetrafluoroethylene/propylene rubbers (FEPM). Fluoroclastomers have five known types. Type 1 FKMs are composed of vinylidene fluoride (VDF) and hexafluoropropylene (HFP) and their fluorine content typically is around 66% by weight. Type 2 FKMs are composed of VDF, HFP, and tetrafluoroethylene (TFE) and typically have between about 68% and 69% fluorine. Type 3 FKMs are composed of VDF, TFE, and perfluoromethylvinylether (PMVE) and typically have between about 62% and 68% fluorine. Type 4 FKMs are composed of propylene, TFE, and VDF and typically have about 67% fluorine. Type 5 FKMs are composed of VDF, HFP, TFE, PMVE, and ethylenc.

In some instances, a substrate disclosed herein comprises a computer readable material. Computer readable materials include, without limitation, magnetic media, reel-to-reel tape, cartridge tape, cassette tape, flexible disk, paper media, film, microfiche, continuous tape (e.g., a belt) and any media suitable for storing electronic instructions. In some instances, the substrate comprises magnetic reel-to-reel tape or a magnetic belt. In some instances, the substrate comprises a flexible printed circuit board.

Structures described herein may be transparent to visible and/or UV light. In some instances, structures described herein are sufficiently conductive to form uniform electric fields across all or a portion of a structure. In some instances, structures described herein are heat conductive or insulated. In some instances, the structures are chemical resistant and heat resistant to support a chemical reaction such as a polynucleotide synthesis reaction. In some instances, the substrate is magnetic. In some instances, the structures comprise a metal or a metal alloy.

Structures for polynucleotide synthesis may be over 1, 2, 5, 10, 30, 50 or more feet long in any dimension. In the case of a flexible structure, the flexible structure is optionally stored in a wound state, e.g., in a reel. In the case of a large structure, e.g., greater than 1 foot in length, the structure can be stored vertically or horizontally.

### Material Deposition Systems

Provided herein are systems and devices for the deposition and storage of biomolecules on a structure described herein. In some instances, the biomolecules are polynucleotides that store encoded information in their sequences. In some instances, the system comprises a surface of a structure to support biomolecule attachment and/or a device for application of a biomolecule to the surface of the substrate. In an example, the device for biomolecule application is a polynucleotide synthesizer. In some instances, the system comprises a device for treating the substrate with a fluid, for example, a flow cell. In some instances, the system comprises a device for moving the substrate between the application device and the treatment device. For instances where the substrate is a reel-to-reel tape, the system may comprise two or more reels that allow for access of different portions of the substrate to the application and optional treatment device at different times.

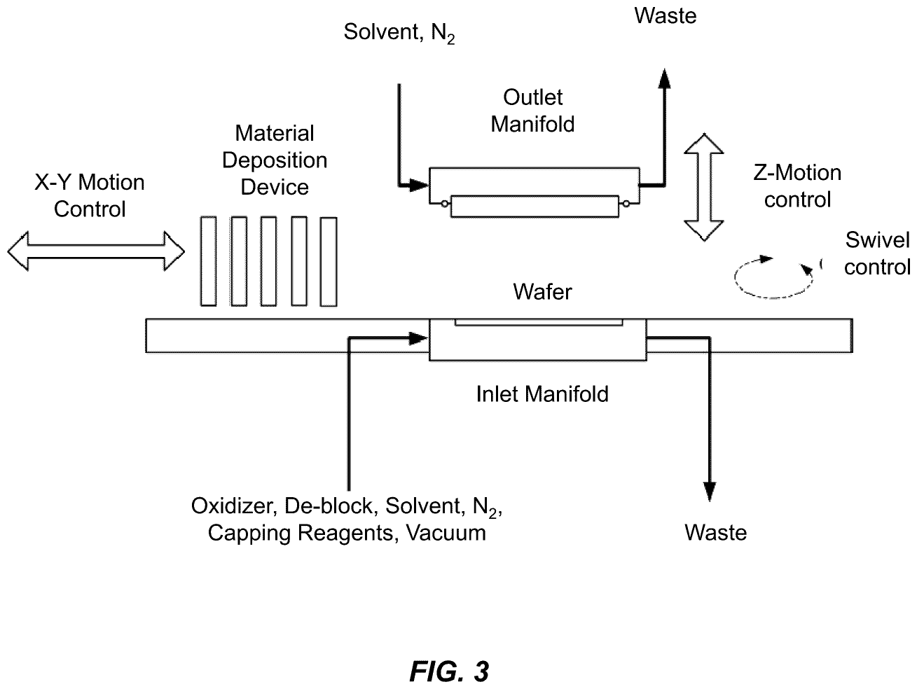

A first example of a polynucleotide material deposition system for polynucleotide synthesis is shown in FIG. 3. The system includes a material deposition device that moves in the X-Y direction to align with the location of the substrate. The material deposition device can also move in the Z direction to seal with the substrate, forming a resolved reactor. A resolved reactor is configured to allow for the transfer of fluid, including polynucleotides and/or reagents, from the substrate to a capping element and/or vice versa. As shown in FIG. 3, fluid may pass through either or both the substrate and the capping element and includes, without limitation, coupling reagents, capping reagents, oxidizers, de-blocking agents, acetonitrile and nitrogen gas. Examples of devices that are capable of high resolution droplet deposition include the printhead of inkjet printers and laser printers. The devices useful in the systems and methods described herein achieve a resolution from about 100 dots per inch (DPI) to about 50,000 DPI; from about 100 DPI to about 20,000 DPI; from about 100 DPI to about 10,000 DPI; from about 100 DPI to about 5,000 DPI; from about 1,000 DPI to about 20,000 DPI; or from about 1,000 DPI to about 10,000 DPI. In some instances, the devices have a resolution at least about 1,000; 2,000; 3,000; 4,000; 5,000; 10,000; 12,000 DPI, or 20,000 DPI. The high resolution deposition performed by the device is related to the number and density of each nozzle that corresponds to a feature of the substrate.

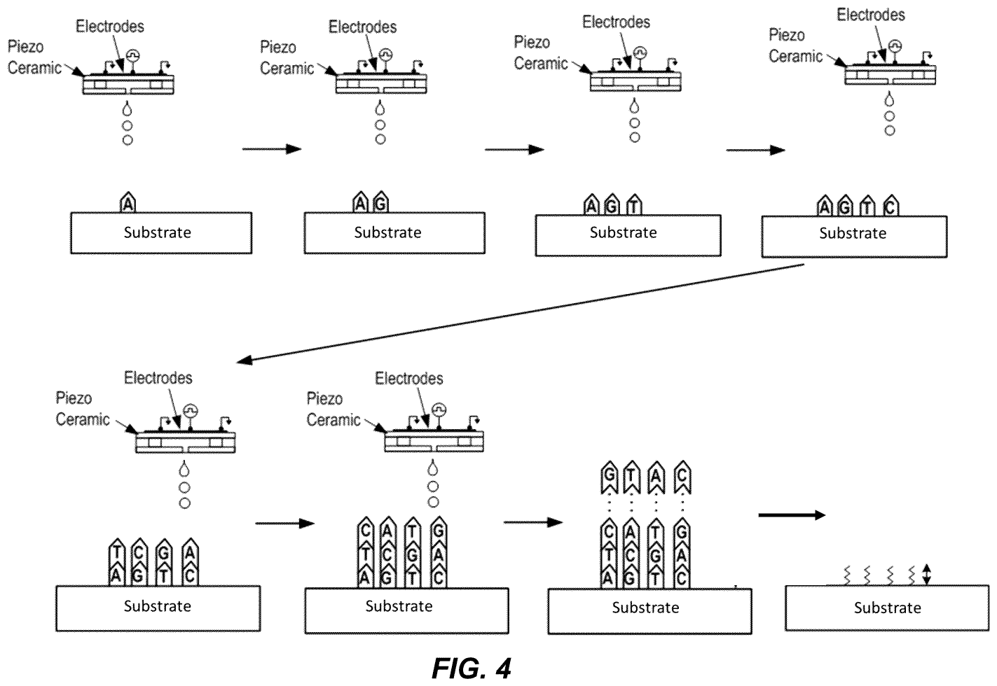

An exemplary process workflow for de novo synthesis of a polynucleotide on a substrate using a polynucleotide synthesizer is shown in FIG. 4. Droplets comprising polynucleotide synthesis reagents are released from the material deposition device to the substrate in a stepwise manner, wherein the material deposition device has a piezo ceramic material and electrodes to convert electrical signals into a mechanical signal for releasing the droplets. The droplets are released to specific locations on the surface of the substrate one nucleobase at a time to generate a plurality of synthesized polynucleotides having predetermined sequences that encode data. In some instances, the synthesized polynucleotides are stored on the substrate. Polynucleotide reagents may be deposited on the substrate surface in a non-continuous, or drop-on-demand method. Examples of such methods include the electromechanical transfer method, electric thermal transfer method, and electrostatic attraction method. In the electromechanical transfer method, piezoelectric elements deformed by electrical pulses cause the droplets to be ejected. In the electric thermal transfer method, bubbles are generated in a chamber of the device, and the expansive force of the bubbles causes the droplets to be ejected. In the electrostatic attraction method, electrostatic force of attraction is used to eject the droplets onto the substrate. In some instances, the drop frequency is from about 5 KHz to about 500 KHz; from about 5 KHz to about 100 KHz; from about 10 KHz to about 500 KHz; from about 10 KHz to about 100 KHz; or from about 50 KHz to about 500 KHz. In some instances, the frequency is less than about 500 KHz, 200 KHz, 100 KHz, or 50 KHz.

The size of the droplets dispensed correlates to the resolution of the device. In some instances, the devices deposit droplets of reagents at sizes from about 0.01 pl to about 20 pl, from about 0.01 pl to about 10 pl, from about 0.01 pl to about 1 pl, from about 0.01 pl to about 0.5 pl, from about 0.01 pl to about 0.01 pl, or from about 0.05 pl to about 1 pl. In some instances, the droplet size is less than about 1 pl, 0.5 pl, 0.2 pl, 0.1 pl, or 0.05 pl. The size of droplets dispensed by the device is correlated to the diameters of deposition nozzles, wherein each nozzle is capable of depositing a reagent onto a feature of the substrate. In some instances, a deposition device of a polynucleotide synthesizer comprises from about 100 to about 10,000 nozzles; from about 100 to about 5,000 nozzles; from about 100 to about 3,000 nozzles; from about 500 to about 10,000 nozzles; or from about 100 to about 5,000 nozzles. In some instances, the deposition device comprises greater than 1,000; 2,000; 3,000; 4,000; 5,000; or 10,000 nozzles. In some instances, each material deposition device comprises a plurality of nozzles, where each nozzle is optionally configured to correspond to a feature on a substrate. Each nozzle may deposit a reagent component that is different from another nozzle. In some instances, each nozzle deposits a droplet that covers one or more features of the substrate. In some instances, one or more nozzles are angled. In some instances, multiple deposition devices are stacked side by side to achieve a fold increase in throughput. In some instances, the gain is 2×, 4×, 8× or more. An example of a deposition device is Samba Printhead (Fujifilm). A Samba Printhead may be used with the Samba Web Administration Tool (SWAT).

The number of deposition sites may be increased by using and rotating the same deposition device by a certain degree or saber angle. By rotating the deposition device, each nozzle is jetted with a certain amount of delay time corresponding to the saber angle. This unsynchronized jetting creates a cross talk among the nozzles. Therefore, when the droplets are jetting at a certain saber angle different from 0 degrees, the droplet volume from the nozzle could be different.

In some arrangements, the configuration of a polynucleotide synthesis system allows for a continuous polynucleotide synthesis process that exploits the flexibility of a substrate for traveling in a reel-to-reel type process. This synthesis process operates in a continuous production line manner with the substrate travelling through various stages of polynucleotide synthesis using one or more reels to rotate the position of the substrate. In an exemplary embodiment, a polynucleotide synthesis reaction comprises rolling a substrate: through a solvent bath, beneath a deposition device for phosphoramidite deposition, through a bath of oxidizing agent, through an acetonitrile wash bath, and through a deblock bath. Alternatively, other wash baths comprising alternative wash solvents are employed, such as acetone, toluene, THF, or other wash solvent. Optionally, the tape is also traversed through a capping bath. A reel-to-reel type process allows for the finished product of a substrate comprising synthesized polynucleotides to be easily gathered on a take-up reel, where it can be transported for further processing or storage.

In some arrangements, polynucleotide synthesis proceeds in a continuous process as a continuous flexible tape is conveyed along a conveyor belt system. Similar to the reel-to-reel type process, polynucleotide synthesis on a continuous tape operates in a production line manner, with the substrate travelling through various stages of polynucleotide synthesis during conveyance. However, in a conveyor belt process, the continuous tape revisits a polynucleotide synthesis step without rolling and unrolling of the tape, as in a reel-to-reel process. In some arrangements, polynucleotide synthesis steps are partitioned into zones and a continuous tape is conveyed through each zone one or more times in a cycle. For example, a polynucleotide synthesis reaction may comprise (1) conveying a substrate through a solvent bath, beneath a deposition device for phosphoramidite deposition, through a bath of oxidizing agent, through an acetonitrile wash bath, and through a block bath in a cycle; and then (2) repeating the cycles to achieve synthesized polynucleotides of a predetermined length. Alternatively, other wash baths comprising alternative wash solvents are employed, such as acetone, THF, or other wash solvent. After polynucleotide synthesis, the flexible substrate is removed from the conveyor belt system and, optionally, rolled for storage. Rolling may be around a reel, for storage.

In an exemplary arrangement, a flexible substrate comprising thermoplastic material is coated with nucleoside coupling reagent. The coating is patterned into features such that each feature has diameter of about 10 μm, with a center-to-center distance between two adjacent features of about 21 μm. In this instance, the feature size is sufficient to accommodate a sessile drop volume of 0.2 pl during a polynucleotide synthesis deposition step. In some instances, the feature density is about 2.2 billion features per m² (1 feature/441×10⁻¹² m²). In some instances, a 4.5 m² substrate comprise about 10 billion features, each with a 10 μm diameter.

In another exemplary arrangement, a substrate comprising nanostructures is coated with nucleoside coupling reagent. The coating is patterned into features such that each feature has diameter of about 10 nm to about 200 nm, with a center-to-center distance between two adjacent features of about 10 nm to about 200 nm. In this instance, a plurality of features accommodates a sessile drop volume of 0.2 pl during a polynucleotide synthesis deposition step. In some instances, a feature diameter of about 50 nm and a center-to-center distance between two adjacent features of about 100 nm results in a feature density of about 10 billion features per m² (1 feature/100×10⁻¹² m²).

A material deposition device described herein may comprises about 2,048 nozzles that each deposit about 100,000 droplets per second at 1 nucleobase per droplet. For each deposition device, at least about 1.75×10¹³ nucleobases are deposited on the substrate per day. In some instances, 100 to 500 nucleobase polynucleotides are synthesized. In some instances, 200 nucleobase polynucleotides are synthesized. Optionally, over 3 days, at a rate of about 1.75×10¹³ bases per day, at least about 262.5×10⁹ polynucleotides are synthesized.

In some arrangements, a device for application of one or more reagents to a substrate during a synthesis reaction is configured to deposit reagents and/or nucleotide monomers for nucleoside phosphoramidite based synthesis. Reagents for polynucleotide synthesis include reagents for polynucleotide extension and wash buffers. As non-limiting examples, the device deposits cleaning reagents, coupling reagents, capping reagents, oxidizers, de-blocking agents, acetonitrile, wash solvents, gases such as nitrogen gas, and any combination thereof. In addition, the device optionally deposits reagents for the preparation and/or maintenance of substrate integrity. In some instances, the polynucleotide synthesizer deposits a drop having a diameter less than about 200 μm, 100 μm, or 50 μm in a volume less than about 1000, 500, 100, 50, or 20 pl. In some instances, the polynucleotide synthesizer deposits between about 1 and 10,000, 1 and 5,000, 100 and 5,000, or 1,000 and 5,000 droplets per second.

In some arrangement, during polynucleotide synthesis, the substrate is positioned within and/or sealed within a flow cell. The flow cell may provide continuous or discontinuous flow of liquids such as those comprising reagents necessary for reactions within the substrate, for example, oxidizers and/or solvents. The flow cell may provide continuous or discontinuous flow of a gas, such as nitrogen, for drying the substrate typically through enhanced evaporation of a volatile substrate. A variety of auxiliary devices are useful to improve drying and reduce residual moisture on the surface of the substrate. Examples of such auxiliary drying devices include, without limitation, a vacuum source, depressurizing pump and a vacuum tank. In some instances, a polynucleotide synthesis system comprises one or more flow cells, such as 2, 3, 4, 5, 6, 7, 8, 9, 10, or 20 and one or more substrates, such as 2, 3, 4, 5, 6, 7, 8, 9, 10 or 20. In some instances, a flow cell is configured to hold and provide reagents to the substrate during one or more steps in a synthesis reaction. In some instances, a flowcell comprises a lid that slides over the top of a substrate and can be clamped into place to form a pressure tight seal around the edge of the substrate. An adequate seal includes, without limitation, a seal that allows for about 1, 2, 3, 4, 5, 6, 7, 8, 9 or 10 atmospheres of pressure. In some instances, the lid of the flow cell is opened to allow for access to an application device such as a polynucleotide synthesizer. In some instances, one or more steps of a polynucleotide synthesis method are performed on a substrate within a flow cell, without the transport of the substrate. The flowcell is optionally used/operated in any orientation, including vertically or horizontally. In some instances, the use of one or more washes in a horizontally orientated flowcell leads to reduced error rates of polynucleotide products.

In some arrangements, a device for treating a substrate with a fluid comprises a spray bar. Nucleotide monomers may be applied onto a substrate surface, and then a spray bar sprays the substrate surface with one or more treatment reagents using spray nozzles of the spray bar. In some arrangements, the spray nozzles are sequentially ordered to correlate with different treatment steps during polynucleotide synthesis. The chemicals used in different process steps may be changed in the spray bar to readily accommodate changes in a synthesis method or between steps of a synthesis method. In some instances, the spray bar continuously sprays a given chemistry on a surface of a substrate as the substrate moves past the spray bar. In some instances, the spray bar deposits over a wide area of a substrate, much like the spray bars used in lawn sprinklers. In some instances, the spray bar nozzles are positioned to provide a uniform coat of treatment material to a given area of a substrate.

In some instances, a polynucleotide synthesis system comprises one or more elements useful for downstream processing of synthesized polynucleotides. As an example, the system comprises a temperature control element such as a thermal cycling device. In some instances, the temperature control element is used with a plurality of resolved reactors to perform polynucleotide assembly such as PCA and/or polynucleotide amplification such as PCR.

In some instances, polynucleotides are synthesized with photolabile protecting groups, where the hydroxyl groups generated on the surface are blocked by photolabile-protecting groups. When the surface is exposed to UV light, such as through a photolithographic mask, a pattern of free hydroxyl groups on the surface may be generated. These hydroxyl groups can react with photoprotected nucleoside phosphoramidites, according to phosphoramidite chemistry. A second photolithographic mask can be applied and the surface can be exposed to UV light to generate second pattern of hydroxyl groups, followed by coupling with 5′-photoprotected nucleoside phosphoramidite. Likewise, patterns can be generated and oligomer chains can be extended. Without being bound by theory, the lability of a photocleavable group depends on the wavelength and polarity of a solvent employed and the rate of photocleavage may be affected by the duration of exposure and the intensity of light. This method can leverage a number of factors such as accuracy in alignment of the masks, efficiency of removal of photo-protecting groups, and the yields of the phosphoramidite coupling step. Further, unintended leakage of light into neighboring sites can be minimized. The density of synthesized oligomer per spot can be monitored by adjusting loading of the leader nucleoside on the surface of synthesis.

The surface of the substrate that provides support for polynucleotide synthesis may be chemically modified to allow for the synthesized polynucleotide chain to be cleaved from the surface. In some instances, the polynucleotide chain is cleaved at the same time as the polynucleotide is deprotected. In some instances, the polynucleotide chain is cleaved after the polynucleotide is deprotected. In an exemplary scheme, a trialkoxysilyl amine such as (CH₃CH₂O)₃Si—(CH₂)₂—NH₂ is reacted with surface SiOH groups of a substrate, followed by reaction with succinic anhydride with the amine to create an amide linkage and a free OH on which the polynucleotide chain growth is supported. Cleavage includes gas cleavage with a base, such as ammonia or methylamine. In some instances, once released from the surface, polynucleotides are assembled into larger polynucleotides that are sequenced and decoded to extract stored information.

Provided herein are systems and methods for synthesis of a high density of polynucleotides on a substrate in a short amount of time. In some instances, the substrate is a flexible substrate. In some instances, at least about 10¹⁰, 10¹¹, 10¹², 10¹³, 10¹⁴, or 10¹⁵ bases are synthesized in one day. In some instances, at least about 10×10⁸, 10×10⁹, 10×10¹⁰, 10×10¹¹, or 10×10¹² polynucleotides are synthesized in one day. In some instances, each polynucleotide synthesized comprises at least about 20, 50, 100, 200, 300, 400 or 500 nucleobases. In some instances, these bases are synthesized with a total average error rate of less than about 1 in 100; 200; 300; 400; 500; 1000; 2000; 5000; 10000; 15000; 20000 bases. In some instances, these error rates are for at least 50%, 60%, 70%, 80%, 90%, 95%, 98%, 99%, 99.5%, or more of the polynucleotides synthesized. In some instances, these at least 90%, 95%, 98%, 99%, 99.5%, or more of the polynucleotides synthesized do not differ from a predetermined sequence for which they encode. In some instances, the error rate for synthesized polynucleotides on a substrate using the methods and systems described herein is less than about 1 in 200. In some instances, the error rate for synthesized polynucleotides on a substrate using the methods and systems described herein is less than about 1 in 1,000. In some instances, the error rate for synthesized polynucleotides on a substrate using the methods and systems described herein is less than about 1 in 2,000. In some instances, the error rate for synthesized polynucleotides on a substrate using the methods and systems described herein is less than about 1 in 3,000. In some instances, the error rate for synthesized polynucleotides on a substrate using the methods and systems described herein is less than about 1 in 5,000. Individual types of error rates include mismatches, deletions, insertions, and/or substitutions for the polynucleotides synthesized on the substrate. The term “error rate” refers to a comparison of the collective amount of synthesized polynucleotide to an aggregate of predetermined polynucleotide sequences. In some instances, synthesized polynucleotides disclosed herein comprise a tether of 12 to 25 bases. In some instances, the tether comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more bases.

A suitable method for polynucleotide synthesis on a substrate of this disclosure is a phosphoramidite method comprising the controlled addition of a phosphoramidite building block, i.e. nucleoside phosphoramidite, to a growing polynucleotide chain in a coupling step that forms a phosphite triester linkage between the phosphoramidite building block and a nucleoside bound to the substrate (for example, an elongation step). In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition and linkage of a nucleoside phosphoramidite in the coupling step, the substrate is optionally washed. In some instances, the coupling step is repeated one or more additional times, optionally with a wash step between nucleoside phosphoramidite additions to the substrate.

### Polynucleotide Assembly

Polynucleotides may be designed to collectively span a large region of a predetermined sequence that encodes for information. In some instances, larger polynucleotides are generated through ligation reactions to join the synthesized polynucleotides. One example of a ligation reaction is polymerase chain assembly (PCA). In some instances, at least of a portion of the polynucleotides are designed to include an appended region that is a substrate for universal primer binding. For PCA reactions, the presynthesized polynucleotides include overlaps with each other (e.g., 4, 20, 40 or more bases with overlapping sequence). During the polymerase cycles, the polynucleotides anneal to complementary fragments and then are filled in by polymerase. Each cycle thus increases the length of various fragments randomly depending on which polynucleotides find each other. Complementarity amongst the fragments allows for forming a complete large span of double-stranded DNA. In some instances, after the PCA reaction is complete, an error correction step is conducted using mismatch repair detecting enzymes to remove mismatches in the sequence. Once larger fragments of a target sequence are generated, they can be amplified. For example, in some instances, a target sequence comprising 5′ and 3′ terminal adapter sequences is amplified in a polymerase chain reaction (PCR) which includes modified primers that hybridize to the adapter sequences. In some instances, the modified primers comprise one or more uracil bases. The use of modified primers allows for removal of the primers through enzymatic reactions centered on targeting the modified base and/or gaps left by enzymes which cleave the modified base pair from the fragment. What remains is a double-stranded amplification product that lacks remnants of adapter sequence. In this way, multiple amplification products can be generated in parallel with the same set of primers to generate different fragments of double-stranded DNA.

Error correction may be performed on synthesized polynucleotides and/or assembled products. An example strategy for error correction involves site-directed mutagenesis by overlap extension PCR to correct errors, which is optionally coupled with two or more rounds of cloning and sequencing. In certain instances, double-stranded polynucleotides with mismatches, bulges and small loops, chemically altered bases and/or other heteroduplexes are selectively removed from populations of correctly synthesized polynucleotides. In some instances, error correction is performed using proteins/enzymes that recognize and bind to or next to mismatched or unpaired bases within double-stranded polynucleotides to create a single or double-strand break or to initiate a strand transfer transposition event. Non-limiting examples of proteins/enzymes for error correction include endonucleases (T7 Endonuclease I, E. coli Endonuclease V, T4 Endonuclease VII, mung bean nuclease, Cell, E. coli Endonuclease IV, UVDE), restriction enzymes, glycosylases, ribonucleases, mismatch repair enzymes, resolvases, helicases, ligases, antibodies specific for mismatches, and their variants. Examples of specific error correction enzymes include T4 endonuclease 7, T7 endonuclease 1, S1, mung bean endonuclease, MutY, MutS, MutH, MutL, cleavase, CELI, and HINF1. In some instances, DNA mismatch-binding protein MutS (Thermus aquaticus) is used to remove failure products from a population of synthesized products. In some instances, error correction is performed using the enzyme Correctase. In some instances, error correction is performed using SURVEYOR endonuclease (Transgenomic), a mismatch-specific DNA endonuclease that scans for known and unknown mutations and polymorphisms for heteroduplex DNA.

### Computer Systems

In various aspects, any of the systems described herein are operably linked to a computer and are optionally automated through a computer either locally or remotely. In various instances, the methods and systems of the invention further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. In some instances, the computer systems are programmed to interface between the user specified base sequence and the position of a material deposition device to deliver the correct reagents to specified regions of the substrate.

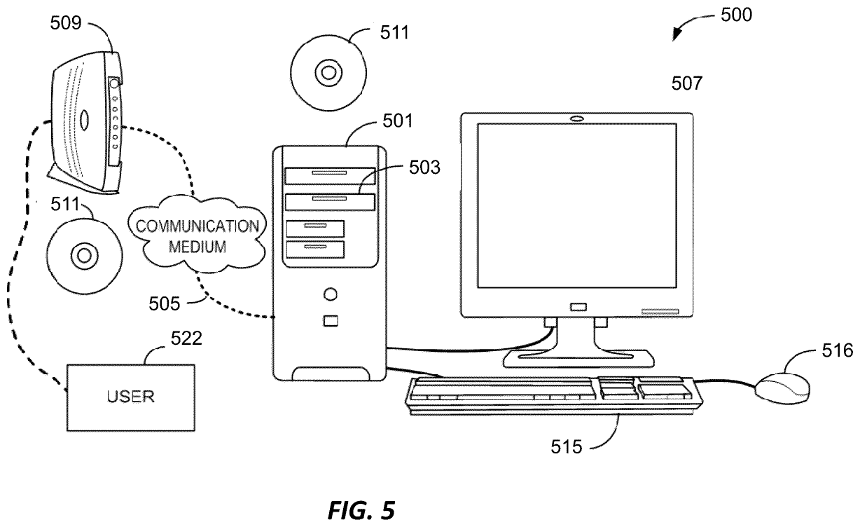

The computer system 500 illustrated in FIG. 5 may be understood as a logical apparatus that can read instructions from media 511 and/or a network port 505, which can optionally be connected to server 509. The system, such as shown in FIG. 5 can include a CPU 501, disk drives 503, optional input devices such as keyboard 515 and/or mouse 516 and optional monitor 507. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 522.

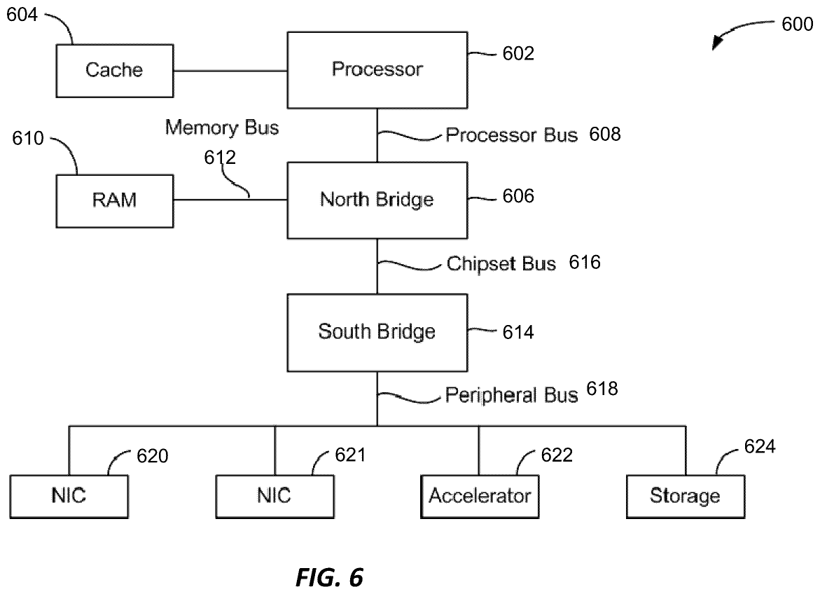

FIG. 6 is a block diagram illustrating a first example architecture of a computer system 1000 that can be used in connection with example instances of the present invention. As depicted in FIG. 6, the example computer system can include a processor 602 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 6, a high speed cache 604 can be connected to, or incorporated in, the processor 602 to provide a high speed memory for instructions or data that have been recently, or are frequently, used by processor 602. The processor 602 is connected to a north bridge 606 by a processor bus 608. The north bridge 606 is connected to random access memory (RAM) 610 by a memory bus 612 and manages access to the RAM 610 by the processor 602. The north bridge 606 is also connected to a south bridge 614 by a chipset bus 616. The south bridge 614 is, in turn, connected to a peripheral bus 618. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 618. In some alternative architectures, the functionality of the north bridge can be incorporated into the processor instead of using a separate north bridge chip.

In some instances, system 600 can include an accelerator card 622 attached to the peripheral bus 618. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 624 and can be loaded into RAM 610 and/or cache 604 for use by the processor. The system 600 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present invention.

In this example, system 600 also includes network interface cards (NICs) 620 and 621 connected to the peripheral bus for providing network interfaces to external storage, such as Network Attached Storage (NAS) and other computer systems that can be used for distributed parallel processing.

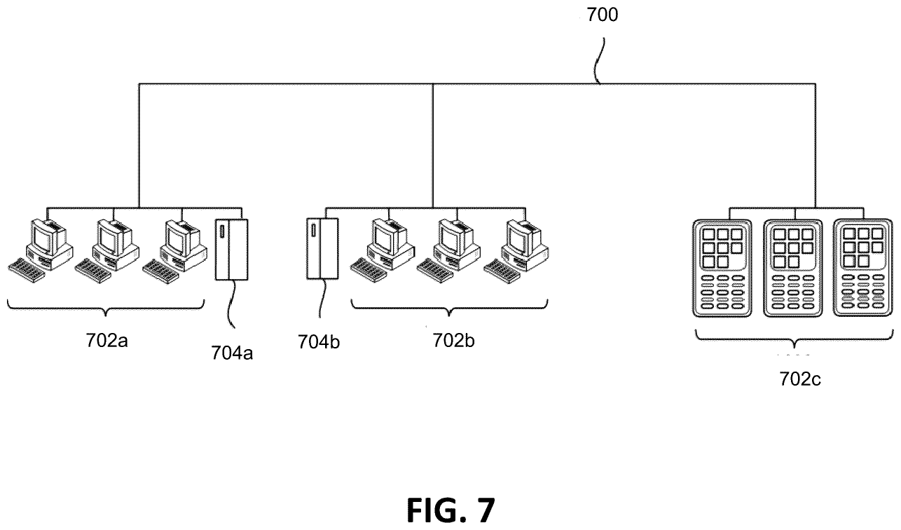

FIG. 7 is a diagram showing a network 700 with a plurality of computer systems 702a, and 702b, a plurality of cell phones and personal data assistants 702c, and Network Attached Storage (NAS) 704a, and 704b. In example instances, systems 702a, 702b, and 702c can manage data storage and optimize data access for data stored in Network Attached Storage (NAS) 704a and 704b. A mathematical model can be used for the data and be evaluated using distributed parallel processing across computer systems 702a, and 702b, and cell phone and personal data assistant systems 702c. Computer systems 702a, and 702b, and cell phone and personal data assistant systems 702c can also provide parallel processing for adaptive data restructuring of the data stored in Network Attached Storage (NAS) 704a and 704b. FIG. 7 illustrates an example only, and a wide variety of other computer architectures and systems can be used in conjunction with the various instances of the present invention. For example, a blade server can be used to provide parallel processing. Processor blades can be connected through a back plane to provide parallel processing. Storage can also be connected to the back plane or as Network Attached Storage (NAS) through a separate network interface.

In some example instances, processors can maintain separate memory spaces and transmit data through network interfaces, back plane or other connectors for parallel processing by other processors. In other instances, some or all of the processors can use a shared virtual address memory space.

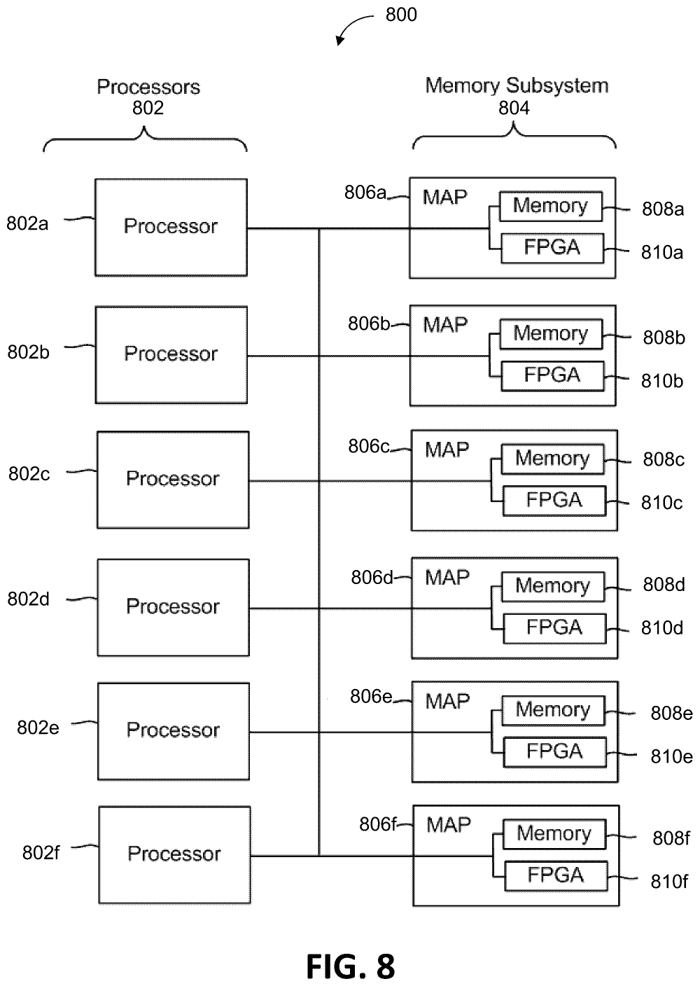

FIG. 8 is a block diagram of a multiprocessor computer system 800 using a shared virtual address memory space in accordance with an example embodiment. The system includes a plurality of processors 802a-f that can access a shared memory subsystem 804. The system incorporates a plurality of programmable hardware memory algorithm processors (MAPs) 806a-f in the memory subsystem 804. Each MAP 806a-f can comprise a memory 808a-f and one or more field programmable gate arrays (FPGAs) 810a-f. The MAP provides a configurable functional unit and particular algorithms or portions of algorithms can be provided to the FPGAs 810a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 808a-f, allowing it to execute tasks independently of, and asynchronously from, the respective microprocessor 802a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

### Embodiments

Provided herein are methods for polynucleotide synthesis, comprising: a) providing a structure comprising a surface; b) coupling at least one nucleoside to a polynucleotide attached to the surface; c) depositing an oxidizing solution on the surface; d) depositing a wash solvent on the surface, wherein the wash solvent comprises a ketone, an ester, an ether, a hydrocarbon, or a functional equivalent thereof; and c) repeating steps b-d to synthesize a plurality of polynucleotides. Further provided herein are methods wherein the method further comprises depositing a capping solution on the surface, wherein capping prevents coupling of unblocked nucleosides. Further provided herein are methods wherein the method further comprises depositing a deblocking solution on the surface, wherein deblocking allows coupling of the polynucleotide to a nucleoside. Further provided herein are methods wherein one or more steps is followed by washing the surface with the wash solvent. Further provided herein are methods wherein one or more steps is followed by washing the surface with acetonitrile. Further provided herein are methods wherein each step subsequent to a coupling step is followed by washing the surface with the wash solvent. Further provided herein are methods wherein each step subsequent to a capping step is followed by washing the surface with the wash solvent. Further provided herein are methods wherein each step subsequent to a deblocking step is followed by washing the surface with the wash solvent. Further provided herein are methods wherein the at least one nucleoside comprises a phosphoramidite. Further provided herein are methods wherein the at least one nucleoside comprises a 5′ blocking group. Further provided herein are methods wherein the at least one nucleoside comprises a 3′ blocking group. Further provided herein are methods wherein the wash solvent is functionally equivalent to a primary constituent by volume of a previously contacted reagent solution. Further provided herein are methods wherein the wash solvent comprises acetone, tetrahydrofuran, ethyl acetate, toluene, benzene, ethanol, or a combination thereof. Further provided herein are methods wherein the wash solvent comprises a ketone, an ether, or a functional equivalent thereof. Further provided herein are methods wherein the structure is a plate, a tape, a belt, or a bead. Further provided herein are methods wherein the oxidizing solution comprises iodine. Further provided herein are methods wherein the polynucleotide or nucleoside comprises DNA or RNA.

Provided herein are methods for polynucleotide synthesis, comprising: a) providing a structure comprising a surface; b) coupling at least one nucleoside to a polynucleotide attached to the surface, wherein each of the at least one nucleosides comprises a blocking group; c) depositing an oxidizing solution on the surface; d) depositing a wash solvent on the surface, wherein the wash solvent comprises acetone or a functional equivalent thereof, or wherein the wash solvent comprises tetrahydrofuran or a functional equivalent thereof; e) depositing a deblocking solution on the surface to remove the blocking group, wherein removal of the blocking group allows coupling of nucleosides to the polynucleotide; and f) repeating steps b-e to synthesize a plurality of polynucleotides. Further provided herein are methods wherein the method further comprises contacting the surface with a capping solution, wherein capping prevents extension of unblocked nucleosides. Further provided herein are methods wherein the wash solvent is functionally equivalent to a primary constituent by volume of a previously contacted reagent solution. Further provided herein are methods wherein one or more steps is followed by washing the surface with the wash solvent. Further provided herein are methods wherein one or more steps is followed by washing the surface with acetonitrile. Further provided herein are methods wherein each step subsequent to a coupling, capping, or deblocking step is followed by washing the surface with the wash solvent. Further provided herein are methods wherein the at least one nucleoside comprises a phosphoramidite. Further provided herein are methods wherein the at least one nucleoside comprises a 5′ blocking group. Further provided herein are methods wherein the at least one nucleoside comprises a 3′ blocking group. Further provided herein are methods wherein the structure is a plate, a tape, a belt, or a bead. Further provided herein are methods wherein the oxidizing solution comprises iodine. Further provided herein are methods wherein the polynucleotide or nucleoside comprises DNA or RNA.

Provided herein are methods for polynucleotide synthesis, comprising: a) providing a structure comprising a surface; b) coupling at least one nucleoside to a polynucleotide attached to the surface; c) depositing an oxidizing solution on the surface; d) depositing a wash solvent on the surface, wherein I₂ or iodine salts have a greater solubility or increased rate of dissolution in the wash solvent compared to acetonitrile; c) repeating steps b-d to synthesize a plurality of polynucleotides. Further provided herein are methods wherein the method further comprises depositing a deblocking solution on the surface, wherein deblocking allows coupling of the polynucleotide to a nucleoside. Further provided herein are methods wherein one or more steps is followed by washing the surface with the wash solvent. Further provided herein are methods wherein one or more steps is followed by washing the surface with acetonitrile. Further provided herein are methods wherein each step subsequent to a coupling or deblocking step is followed by washing the surface with the wash solvent. Further provided herein are methods wherein the wash solvent is functionally equivalent to a primary constituent by volume of a previously contacted reagent solution. Further provided herein are methods wherein the at least one nucleoside comprises a phosphoramidite. Further provided herein are methods wherein the at least one nucleoside comprises a 5′ blocking group. Further provided herein are methods wherein the at least one nucleoside comprises a 3′ blocking group. Further provided herein are methods wherein the structure is a plate, a tape, a belt, or a bead. Further provided herein are methods wherein the oxidizing solution comprises iodine. Further provided herein are methods wherein the oxidizing solution further comprises an amine base. Further provided herein are methods wherein the amine base is selected from pyridine, lutidine, collidine, N-methyl morpholine, or a functional equivalent thereof.

Further provided herein are methods wherein the polynucleotide or nucleoside comprises DNA or RNA.

Provided herein are methods of synthesizing polynucleotides, comprising: a) providing predetermined sequences for a plurality of preselected polynucleotides; b) providing a substrate comprising a surface; c) synthesizing the plurality of preselected polynucleotides extending from the surface, wherein synthesizing comprises one or more wash steps with a wash solvent after depositing an oxidizing solution on the surface, wherein the wash solvent dissolves an active component or byproduct of the oxidizing solution, wherein the wash solvent comprises a ketone, an ester, an ether, a hydrocarbon, or a functional equivalent thereof; and d) assembling the plurality of preselected polynucleotides. Further provided herein are methods wherein the method further comprises washing before or after depositing the oxidizing solution on the surface, wherein washing comprises depositing the wash solvent on the surface. Further provided herein are methods wherein the wash solvent comprises a ketone, ether, or a functional equivalent thereof. Further provided herein are methods wherein the wash solvent comprises acetone, tetrahydrofuran, or a functional equivalent thereof. Further provided herein are methods wherein the oxidizing solution comprises iodine.

Further provided herein are methods wherein the oxidizing solution further comprises an amine base. Further provided herein are methods wherein the amine base is selected from pyridine, lutidine, collidine, N-methyl morpholine, or a functional equivalent thereof. Further provided herein are methods wherein the polynucleotide or nucleoside comprises DNA or RNA.

Provided herein are methods for polynucleotide synthesis, comprising: a) providing a structure comprising a surface; b) coupling at least one nucleoside to a polynucleotide attached to the surface; c) depositing an oxidizing solution on the surface; d) depositing a capping solution on the surface before or after depositing the oxidizing solution on the surface; c) repeating steps b-d to synthesize a plurality of polynucleotides. Further provided are methods further comprising depositing the capping solution on the surface before and after depositing the oxidizing solution on the surface. Further provided are methods further comprising depositing a wash solvent after (i) coupling the at least one nucleoside to the polynucleotide attached to the surface; (ii) depositing the capping solution; and (iii) depositing the oxidizing solution. Further provided are methods wherein the wash solvent deposited after depositing the oxidizing solution comprises a ketone, an ester, an ether, a hydrocarbon, or a functional equivalent thereof. Further provided are methods wherein the wash solvent deposited after depositing the oxidizing solution comprises a ketone, ether or a functional equivalent thereof. Further provided are methods wherein the wash solvent deposited after depositing the oxidizing solution comprises acetone, THF or a functional equivalent thereof. Further provided are methods wherein the wash solvent deposited after the coupling step or the capping step comprises acetonitrile. Further provided are methods wherein the wash solvent deposited after the coupling step or the capping step comprises acetone, tetrahydrofuran, or a functional equivalent thereof. Further provided are methods wherein the wash solvent comprises a ketone, an ester, an ether, a hydrocarbon, or a functional equivalent thereof. Further provided are methods wherein the wash solvent comprises a ketone, an ether, or a functional equivalent thereof. Further provided are methods wherein the wash solvent comprises acetone, tetrahydrofuran, or a functional equivalent thereof. Further provided are methods wherein the capping solution comprises an acid halide or an anhydride. Further provided are methods wherein the capping solution comprises acetyl chloride or acetic anhydride. Further provided are methods wherein the capping solution comprises an amine base. Further provided are methods wherein the polynucleotide or nucleoside comprises DNA or RNA.

The following examples are set forth to illustrate more clearly the principle and practice of instances disclosed herein to those skilled in the art and are not to be construed as limiting the scope of any claimed instances. Unless otherwise stated, all parts and percentages are on a weight basis.

### EXAMPLES

### Example 1: Polynucleotide Synthesis with Acetone Wash Steps

A substrate surface (such as a silicon plate) was functionalized with 3-glycidoxypropyltrimethoxysilane (GOPS), a chemical group capable of reacting with a phosphoramidite to support the attachment and synthesis of a library of polynucleotides. Synthesis of polynucleotides was accomplished through iteration of the following steps: 1) extension of a plurality of polynucleotides from the surface by contacting the surface with a base addition solution comprising at least one reactive monomer, wherein the at least one reactive monomer was a 3′ phosphoramidite nucleobase (A, T, G, or C) comprising a 5′ blocking group; 2) washing the surface at least once; 3) contacting the surface with an oxidizing solution comprising iodine; 4) washing the surface at least once; and 5) removing 5′ blocking group with a deblocking solution; and 6) washing the surface at least once. Steps 1-6 were repeated until the plurality of polynucleotides were synthesized.

Two different sets of conditions were used with the above polynucleotide synthesis method. In conditions A, washing steps were completed using acetone as the wash reagent. In conditions B, washing steps were completed using acetonitrile as the wash reagent. Polynucleotide library error rates for both conditions were analyzed with Next Generation Sequencing, and the results by error type are shown in Table 5. Error rates were decreased for Conditions A, with the largest percent decrease observed for single base deletions.

TABLE 5
Comparison of error rates for Conditions A and B.

| Error Type | Conditions A | Conditions B |
|---|---|---|
| Insertion Rate | 0.000227 | 0.000315 |
| Mismatch Rate | 0.000798 | 0.000905 |
| Single Base Deletion Rate | 0.000539 | 0.001117 |
| Total Error Rate | 0.001564 | 0.002337 |

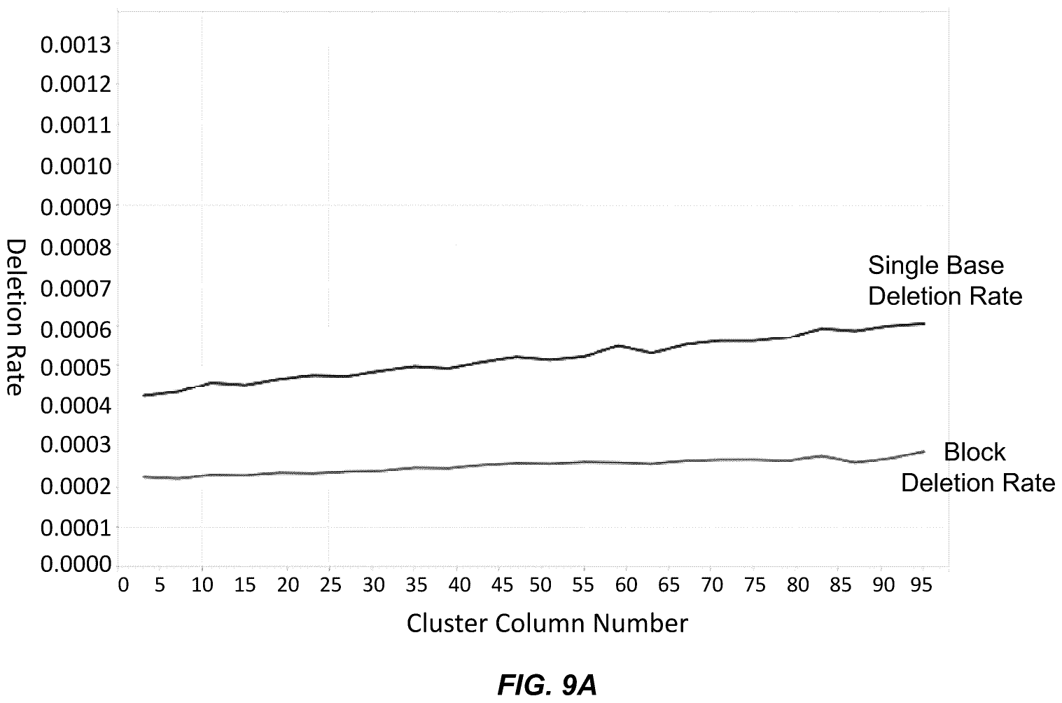

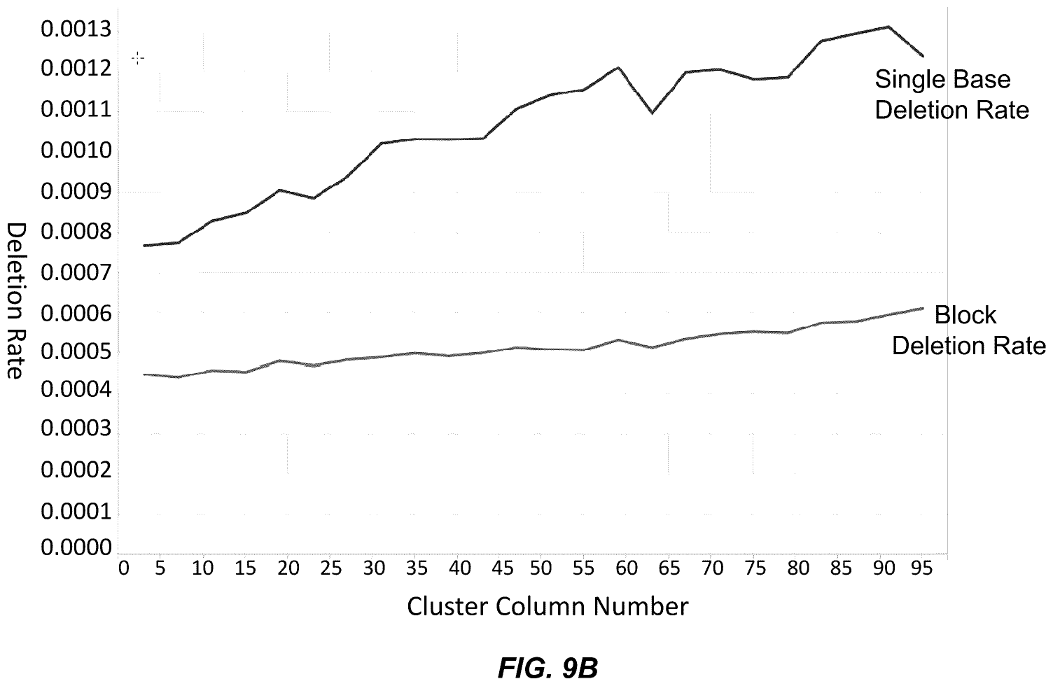

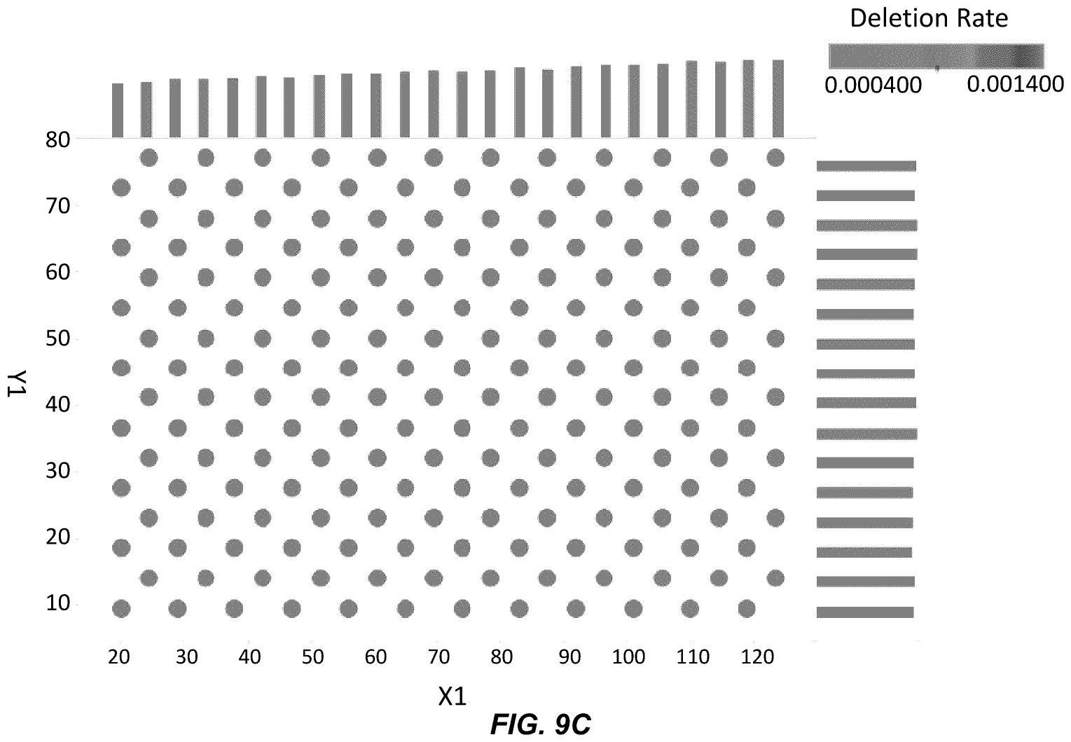

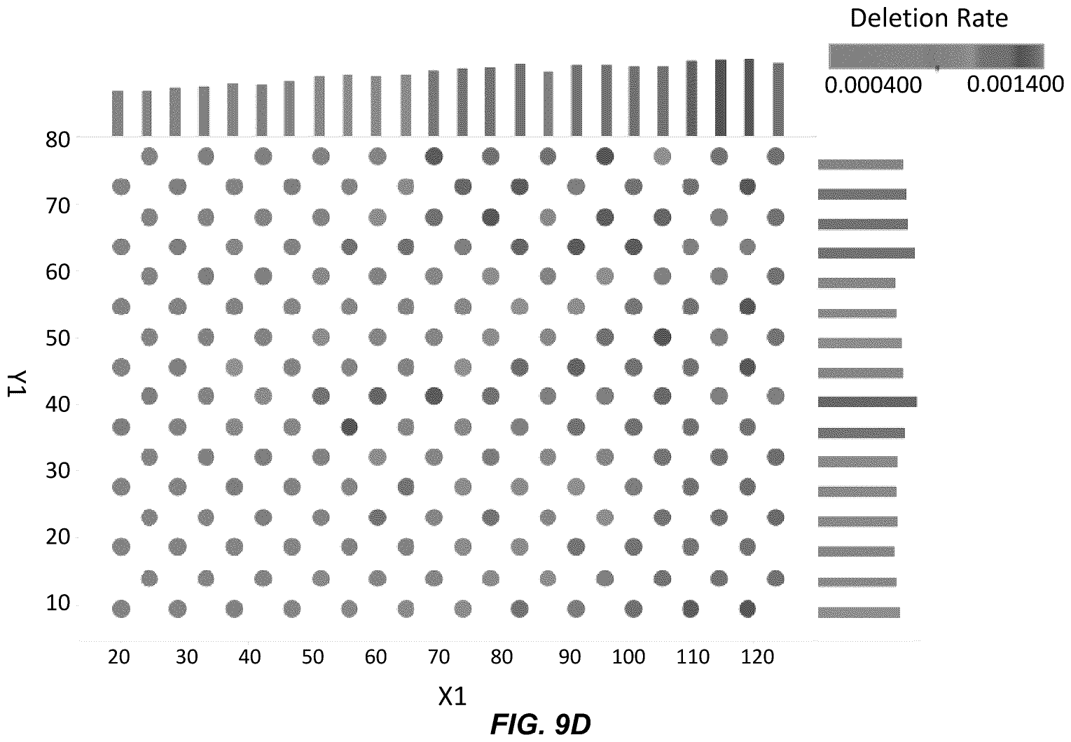

Error rates for single base deletions and block deletions were further examined as a function of columns across the surfaces, wherein each column comprises a series of clusters. Condition A (FIG. 9A) provided lower deletion rates than Condition B (FIG. 9B), and the uniformity of deletions across cluster columns was higher for Conditions A. An examination of all clusters on the surface (plate) also demonstrated higher uniformity of single base deletion rates for Condition A (FIG. 9C) vs. Condition B (FIG. 9D) across clusters on the surface.

### Example 2: Synthesis of Polynucleotides Using an Acetone Wash after Oxidation

A two dimensional polynucleotide synthesis device was assembled into a flowcell, which was connected to a flowcell (Applied Biosystems (“ABI394 DNA Synthesizer”). The surface of the polynucleotide synthesis device was functionalized with 3-glycidoxypropyltrimethoxysilane (GOPS), and was used to synthesize a library of polynucleotides using the synthesis methods described herein. The generic sequence of the nucleic acid synthesized on the surface was 5′-[polynucleotide] ##[TTTTTTTTTT]-3′, where #denotes Thymidine-succinyl hexamide CED phosphoramidite (CLP-2244 from ChemGenes), which is a cleavable linker enabling the release of the polynucleotides from the surface during deprotection. The 3′ end of the polynucleotide was attached to the surface, and the TTTTTTTTTT sequence between the linker and the surface represents a polynucleotide spacer. Synthesis of the spacer region was performed by iteration of the following steps: 1) extension of a plurality of reactive molecules from the surface (initially GOPS, then nucleic acids or polynucleotides in subsequent steps) by contacting the surface with a base addition solution comprising at least one reactive monomer, wherein the at least one reactive monomer was a 3′ phosphoramidite thymine comprising a 5′ blocking group; 2) capping unreacted 5′ OH groups of the thymine by contacting the surface with a capping solution; 3) washing the surface at least once with acetone; 4) contacting the surface with an oxidizing solution comprising iodine; 5) washing the surface at least once with acetone; 6) capping unreacted 5′ OH groups of the thymine by contacting the surface with a capping solution; 7) removing 5′ blocking group with a deblocking solution; and 8) washing the surface at least once with acetone. Steps 1-8 were repeated until the plurality of polynucleotide spacers were synthesized. The cleavable linker group (##) was then coupled to the 5′ end of the polynucleotide spacer, followed by washing the surface at least once with acetone.

Extension beyond the linker/spacer region to synthesize polynucleotides was performed by iteration of the following steps: 1) contacting the surface with a base addition solution comprising at least one reactive monomer, wherein the at least one reactive monomer was a 3′ phosphoramidite nucleobase (A, T, G, or C) comprising a 5′ blocking group; 2) contacting the surface with an oxidizing solution comprising iodine; 3) washing the surface at least once with acetone; and 4) removing 5′ blocking group with a deblocking solution; and 5) washing the surface at least once with acetone. Steps 1-5 were repeated until the plurality of polynucleotides were synthesized. After polynucleotide synthesis, polynucleotides were cleaved from the surface by deprotection in gaseous ammonia overnight for further analysis.

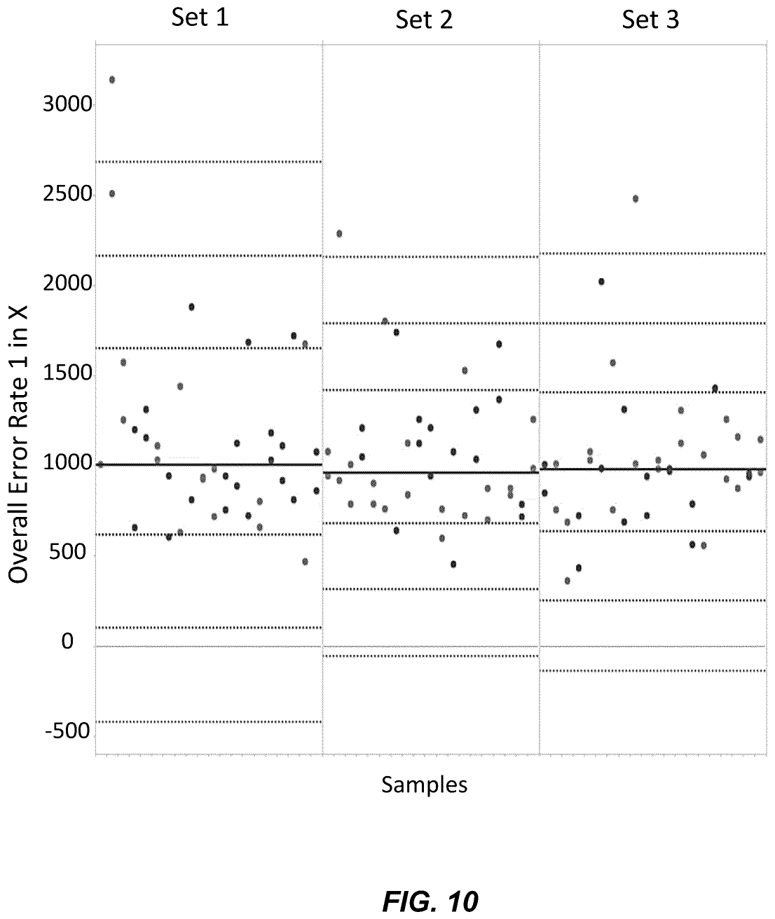

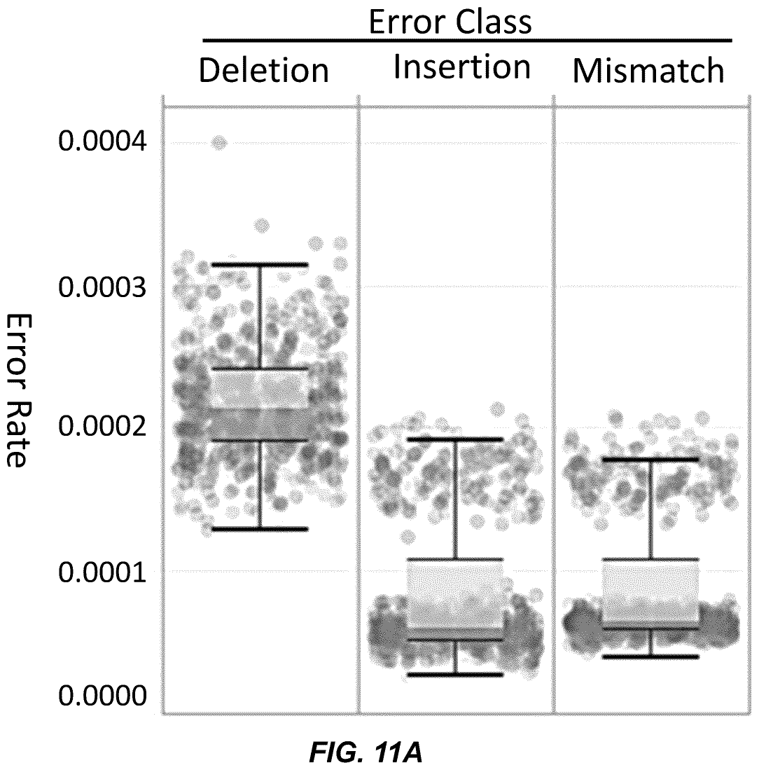

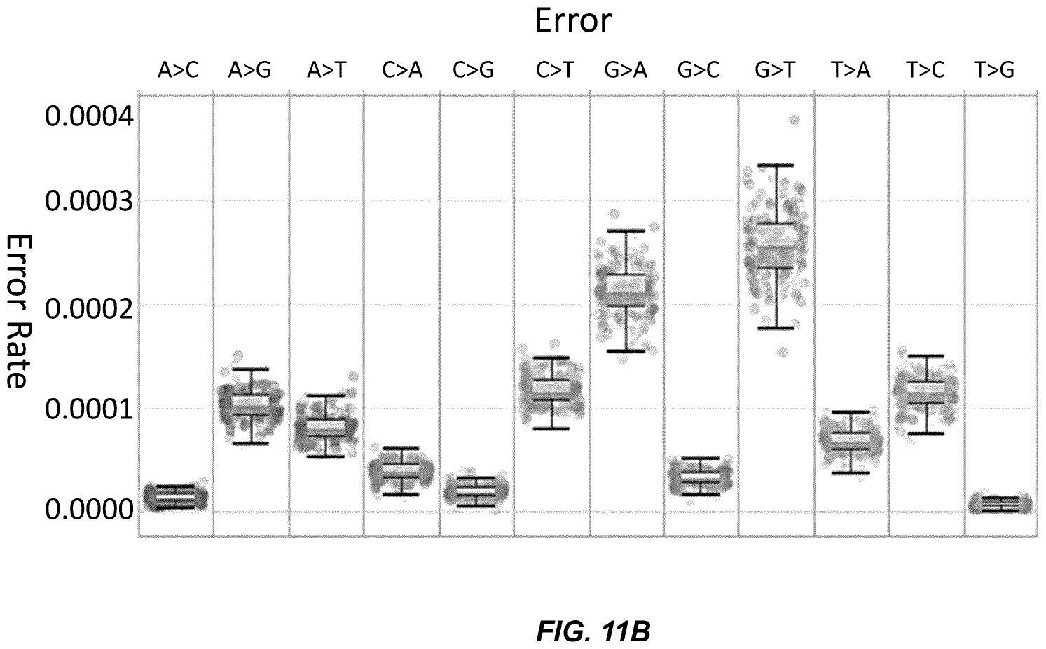

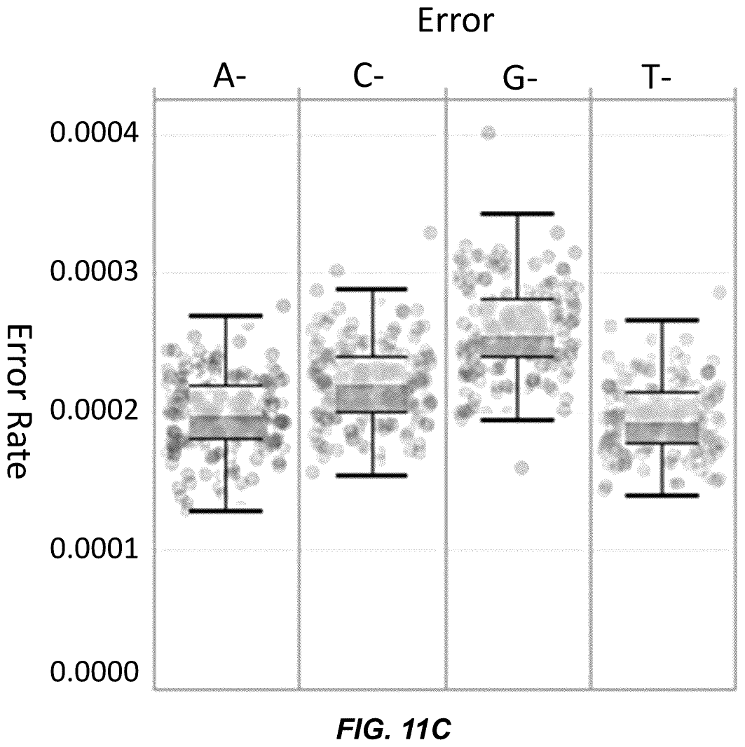

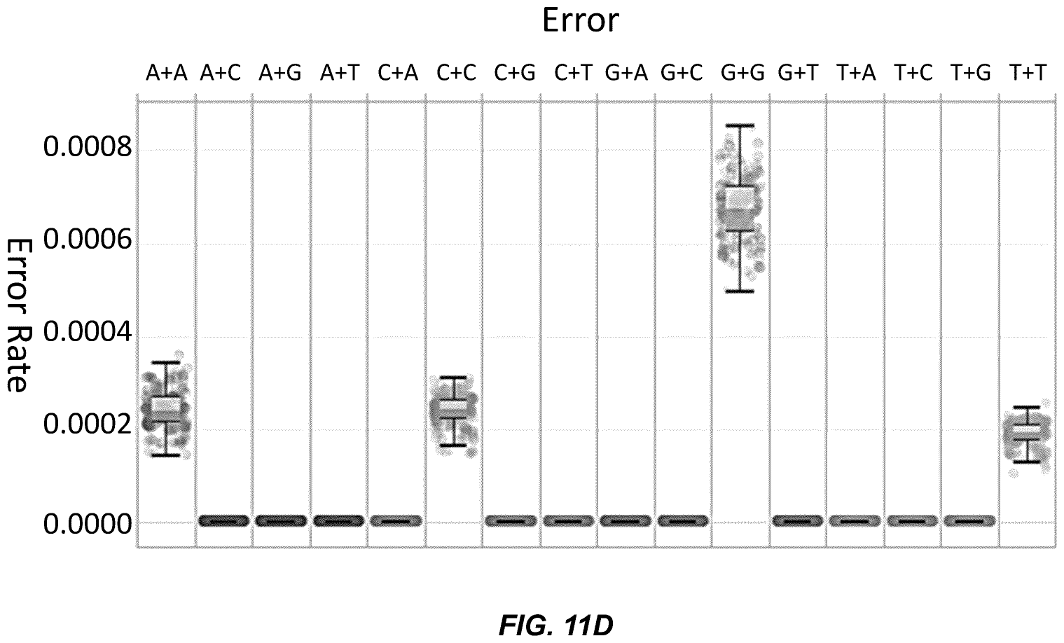

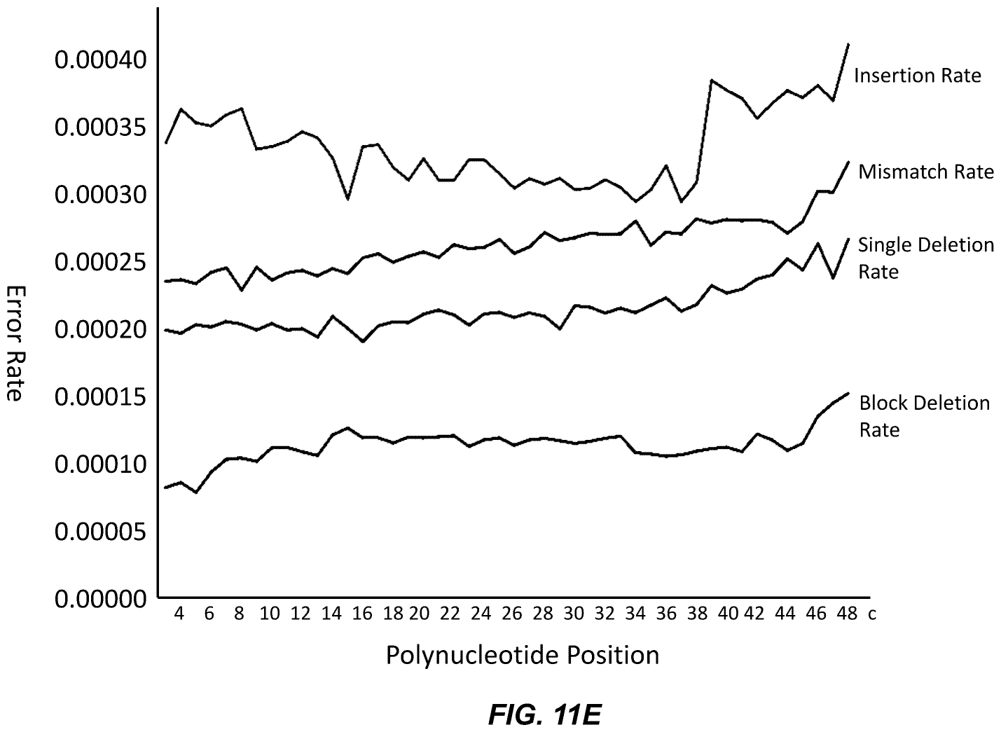

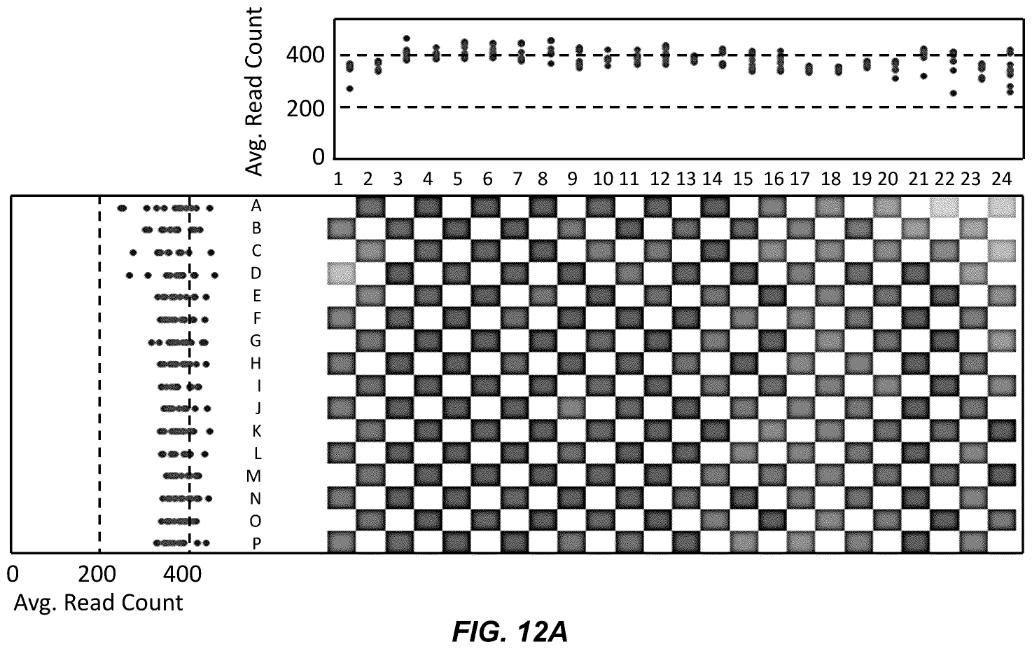

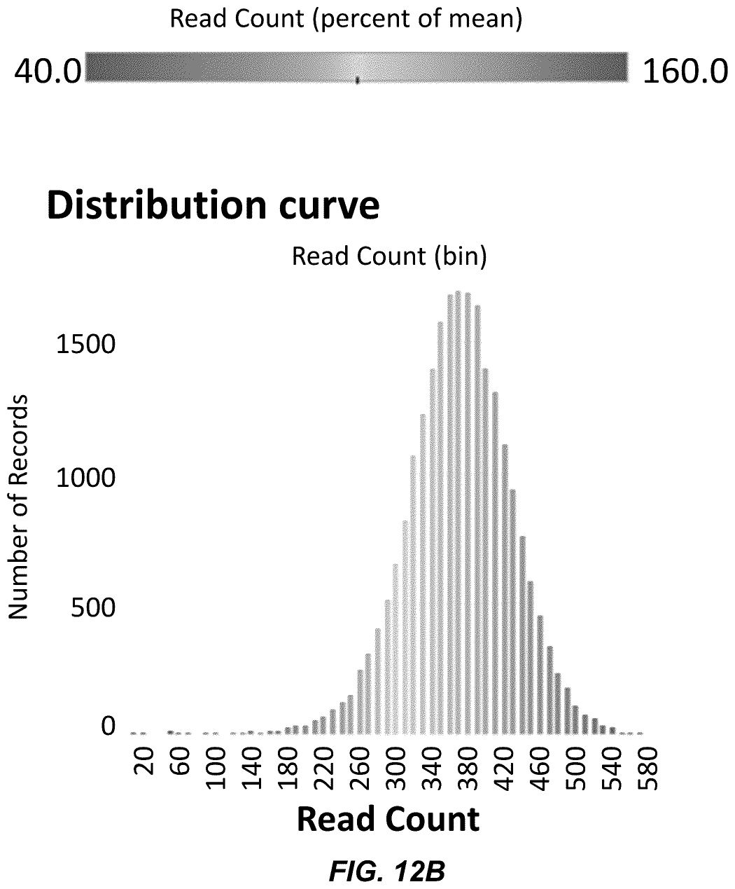

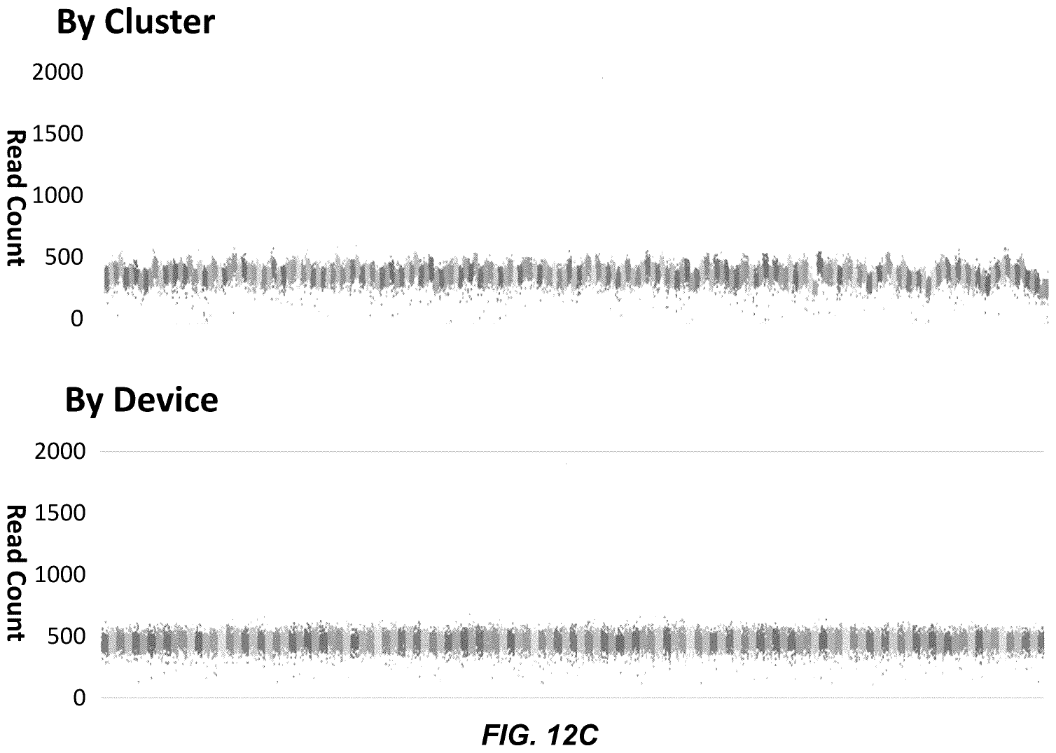

Polynucleotide libraries were synthesized at high uniformity. Several methods were used to evaluate the quality of the polynucleotide library, including Sanger sequencing and Next generation sequencing. The overall error rates for three different sets of oligonucleotide libraries were about 1/1000 or less (FIG. 10). Next generation sequencing was used to evaluate the quality of the polynucleotide library. Error rates for error classes deletions, insertions, and mismatches were measured (FIG. 11A). For example, substitution rates were further evaluated as function of a reference base verses observed base. Substitutions occurred at a uniform rate, with a slightly increased rate of substitution for G to A and G to T substitutions. (FIG. 11B). Deletion rates for each base type were measured; rates were comparable for all bases, about 0.002 (FIG. 11C). G+G insertions increased verses A+A, C+C, and T+T insertion errors (FIG. 11D). Insertion, mismatch, single deletion and block deletion rates were also evaluated as a function of base position on the polynucleotide. Observed error rates were uniform across all positions in the synthesized polynucleotide, with x axis correlating to bases distanced from the surface (FIG. 11E). To evaluate dropouts, clusters from each area of the plate (FIG. 12A) were evaluated for average read count, with a total of 8.7 million reads (Table 6). Mean read counts across all clusters were about 400, and were highly uniform in both the x (1-24) and y (A-P) directions of the plate. The dropout rate was less than 0.0%, with a uniformity of 1.7× of the mean in the distribution curve in FIG. 12B. In another depiction of error rate, read count was highly uniform across clusters (FIG. 12C, top) and across multiple devices (FIG. 12C, bottom).

TABLE 6
Polynucleotide Library Measurements

| Metric | Value |
|---|---|
| Dropouts | 0.0% |
| Uniformity | 1.7x |
| Total Reads | 8.7 million |
| Mean ± SD | 374 ± 61 |

### Example 3: Wash Solutions to Remove Undesired G→A and T/U→C Mutations

A wash solution was prepared as described herein comprising THE, pyridine, and water in an 80:20:10 ratio. Following the general procedure of Examples 1 and 2, phosphoramidite chemistry was used to synthesize samples of polynucleotides. Sample A used a post-coupling wash with the wash solution comprising THF/pyridine/water, followed by acetone, and a second sample B utilized a post-coupling wash of acetone. Deprotection of the first sample with methylamine and a wash step with the wash solution comprising THF, pyridine, and the O-nucleophile (water) resulted in no T→C and G→A mutation rate above the noise level (rates less than 1/5000), as detected by NGS. Deprotection of the second sample with methylamine and a wash step with acetonitrile or acetone resulted in T→C and G→A mutation rates greater than 1/1500. Uniformity of syntheses carried out using the wash solution was highly improved. The results showed complete removal, within the detection limit, of the phosphoramidite/tetrazole material deposited in the previous coupling reaction by printing. Uniformity of the resulting polynucleotides was improved for Sample A. For sample A, approximately >90% of all records had between 220 and 670 reads. For sample B, approximately >90% of all records had between 30 and 940 reads.

TABLE 7
Wash solution results

| | | | Total Reads | |
|---|---|---|---|---|
| Sample | Dropouts (%) | Uniformity | (millions) | Mean ± SD |
| A | 0.1 | 6.9 | 12 | 518 ± 229 |
| B | 0.1 | 1.9 | 10.1 | 436 ± 88 |

### Example 4: Capping to Remove Undesired G→A and T/U→C Mutations

A capping solution 1 was prepared as described herein comprising THF, lutidine, acetic anhydride in an 80:10:10 ratio, with added acetic acid (10 g/L). A capping solution 2 comprising acetonitrile and n-methylimidazole in a 90:10 ratio was also prepared. Following the general procedure of Examples 1 and 2, phosphoramidite chemistry was used to synthesize samples of polynucleotides. Sample A was washed a mix of solutions 1 and 2, and a second sample B was washed using a capping solution in the absence of the acetic acid. Deprotection of the first sample with methylamine and a wash step with the capping solution comprising the O-nucleophile (acetic acid) resulted in no T→C and G→A mutation rate above the noise level (rates of 1/10101), as detected by NGS. Deprotection of the second sample with methylamine and a wash step with a capping solution without the O-nucleoside resulted in T→C and G→A mutation rates of 1/3058. The addition of alternative O-nucleophiles such as sigma-acceptor-substituted acetic acid derivatives, such as methoxyacetic acid and phenoxyacetic acid, also increased the efficiency of the capping reaction.

### Example 5: Capping Order

Following the general procedures of Examples 1 and 2, sample A was washed with a capping solution after oxidation, and sample B was washed with a capping solution before oxidation. For sample A, the procedure was Wash-Acetone-Ox-Acetone-CapMix (from Example 4)-Acetone-Deblock-Acetone. Sample B had a single base deletion rate of 1/1196, while Sample A had a single base deletion rate of 1/5450.

### Example 6: Base Protected Phosphoramidites

Protected dT and dG phosphoramidite building blocks A-I containing a protecting group at nitrogen atoms N3 and N1, were incorporated into polynucleotides using the general procedure of Examples 1 and 2. After deprotection of the resulting polynucleotide products with MeNH₂, no T→C and G→A mutation above the noise-level (rates less than 1/5000) could be detected by the limits of NGS, whereas a control experiment using standard, unprotected nucleoside phosphoramidites resulted in mutation rates of greater than 1/1500 were obtained. The use of base protected nucleoside phosphoramidites led to lower error rates.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-64. (canceled)

65. A method of polynucleotide synthesis comprising:
providing a structure comprising a surface;
coupling a nucleoside to a polynucleotide attached to the surface;
depositing an oxidizing solution on the surface;
depositing a wash solution on the surface, wherein the wash solution comprises water, a base, and a solvent.

66. The method of claim 65, further comprising repeating the coupling, oxidizing, and washing steps to synthesize a plurality of polynucleotides.

67. The method of claim 65, wherein the wash solution is deposited before the oxidizing solution.

68. The method of claim 65, wherein the wash solution is deposited after the nucleoside is coupled to the polynucleotide.

69. The method of claim 65, wherein the wash solution is a first wash solution, and the method further comprises depositing a second wash solution on the surface, wherein the second wash solution comprises acetone.

70. The method of claim 65, wherein the base includes pyridine, lutidine, or collidine.

71. The method of claim 65, wherein the wash solution includes the base at a concentration of 0.01 M to 3 M.

72. The method of claim 65, wherein the solvent includes acetonitrile, acetone, or THF.

73. The method of claim 65, wherein a ratio of the solvent to the base to the water in the wash solvent is 80:20:10.

74. A method for polynucleotide synthesis, the method comprising:
providing a structure comprising a surface;
coupling a nucleoside to a polynucleotide attached to the surface, wherein the nucleoside comprises a blocking group;
depositing an oxidizing solution on the surface;
depositing a wash solvent on the surface, wherein I₂ or iodine salts have a greater solubility or increased rate of dissolution in the wash solvent compared to acetonitrile; and
depositing a deblocking solution on the surface, wherein depositing the deblocking solution on the surface removes the blocking group.

75. The method of claim 74, wherein the wash solvent comprises acetone.

76. The method of claim 74, wherein the nucleoside is a first nucleoside, the method further comprises:
coupling a second nucleoside to the polynucleotide attached to the surface, wherein the second nucleoside comprises the blocking group;
depositing the wash solvent on the surface;
depositing the oxidizing solution on the surface; and
depositing the deblocking solution on the surface.

77. The method of claim 74, further comprising contacting the surface with a capping solution, wherein the capping solution prevents extension of unblocked nucleosides.

78. The method of claim 74, wherein the wash solvent is deposited on the surface after the nucleoside is coupled to the polynucleotide and prior to depositing the oxidizing solution on the surface, and the method further comprises:
after depositing the oxidizing solution on the surface and prior to depositing the deblocking solution on the surface, depositing the wash solvent on the surface; and
after depositing the deblocking solution on the surface, depositing the wash solvent on the surface.

79. The method of claim 74, wherein the nucleoside comprises a phosphoramidite.

80. The method of claim 74, wherein the blocking group is a 5′ blocking group or a 3′ blocking group.

81. The method of claim 74, wherein the structure includes a plate, a tape, or a belt.

82. The method of claim 74, wherein the oxidizing solution comprises iodine.

83. The method of claim 74, wherein the oxidizing solution further comprises pyridine, lutidine, collidine, N-methyl morpholine, or other amine base.

84. The method of claim 74, wherein the wash solvent dissolves an active component or byproduct of the oxidizing solution.
